(19) United States
(12) Patent Application Publication
(10) Pub. No.: US 2020/0262984 A1
(43) Pub. Date: Aug. 20, 2020

(54) UNIQUE MORPHOLOGICAL POLYSACCHARIDE

(71) Applicant: DuPont Industrial Biosciences USA, LLC, Wilmington, DE (US)

(72) Inventors: Juan David Londono, Bear, DE (US); Natnael Behabtu, Wilmington, DE (US); David M. Scott, Wilmington, DE (US); Yefim Brun, Wilmington, DE (US)

(21) Appl. No.: 16/761,563

(22) PCT Filed: Nov. 6, 2018
(86) PCT No.: PCT/US2018/059354

§ 371 Date: May 5, 2020

Related U.S. Application Data
(60) Provisional application No. 62/584,150, filed on Nov. 10, 2017.

(51) Int. Cl. C08J 3/05; C12P 19/18

(52) U.S. Cl. CPC C08J 3/05; C08J 2305/00; C12Y 204/01; C12P 19/18

(57) ABSTRACT

Disclosed herein are compositions comprising aggregates of insoluble alpha-glucan with alpha-1,3-glycosidic linkages. These aggregates have an average hydrodynamic radius of about 50 nm to about 300 nm, and a fractal dimension of about 1.6 to about 2.4. Insoluble alpha-glucan aggregates in some disclosed aspects are arborescent. Further disclosed are methods of preparing these compositions.

This application claims the benefit of U.S. Provisional Application No. 62/584,150 (filed Nov. 10, 2017), which is incorporated herein by reference in its entirety.

### FIELD

The present disclosure is in the field of polysaccharide materials. For example, the disclosure pertains to compositions comprising aggregates of insoluble alpha-glucan with unique morphology.

### REFERENCE TO SEQUENCE LISTING SUBMITTED ELECTRONICALLY

The official copy of the sequence listing is submitted electronically via EFS-Web as an ASCII formatted sequence listing with a file named 20181106_CL6617WOPCT_SequenceListing.txt, created on Nov. 6, 2018, and having a size of about 315 kilobytes and is filed concurrently with the specification. The sequence listing contained in this ASCII-formatted document is part of the specification and is herein incorporated by reference in its entirety.

### BACKGROUND

Driven by a desire to use polysaccharides in various applications, researchers have explored for polysaccharides that are biodegradable and that can be made economically from renewably sourced feedstocks. One such polysaccharide is alpha-1,3-glucan, an insoluble glucan polymer characterized by having alpha-1,3-glycosidic linkages. This polymer has been prepared, for example, using a glucosyltransferase enzyme isolated from Streptococcus salivarius (Simpson et al., Microbiology 141:1451-1460, 1995). Also for example, U.S. Pat. No. 7,000,000 disclosed the preparation of a spun fiber from enzymatically produced alpha-1,3-glucan. Various other glucan materials have also been studied for developing new or enhanced applications. For example, U.S. Patent Appl. Publ. No. 2015/0232819 discloses enzymatic synthesis of several insoluble glucans having mixed alpha-1,3 and -1,6 linkages.

Despite this work, new forms of insoluble alpha-glucan are desired to enhance the economic value and performance characteristics of this material in various applications. Compositions comprising insoluble alpha-glucan aggregates with unique morphological features are presently disclosed to address this need.

### SUMMARY

In one embodiment, the present disclosure concerns a composition comprising aggregates of insoluble alpha-glucan, wherein the aggregates have an average hydrodynamic radius of about 50-300 nm and a fractal dimension of about 1.6-2.4, and the insoluble alpha-glucan comprises alpha-1,3-glycosidic linkages.

In another embodiment, the present disclosure concerns a method of producing aggregates of insoluble alpha-glucan herein, the method comprising: (a) contacting at least water, sucrose, and a glucosyltransferase enzyme that synthesizes insoluble alpha-glucan at a yield of at least about 40%; and (b) preparing a dispersion of the insoluble alpha-glucan produced in step (a). The present disclosure also concerns a composition comprising aggregates of insoluble alpha-glucan produced according to this method.

### BRIEF DESCRIPTION OF THE DRAWINGS AND SEQUENCES

FIG. 1: Typical arborescent structures from computer simulations of aggregates formed by diffusion of primary spherical particles. The fractal dimension of each structure is provided. Such structures are contemplated in some aspects to be formed, at least in part, from aggregates of primary rod-like particles.

TABLE 1
Summary of Nucleic Acid and Protein SEQ ID Numbers^{b}

| | Nucleic acid | Protein | |
|---|---|---|---|
| Description | SEQ ID NO. | SEQ ID NO. | |
| GTF 0874, Streptococcus sobrinus. The first 156 amino acids | 1 ^{a} | 2 | (1435 aa) |
| of the protein are deleted compared to GENBANK | | | |
| Identification No. 450874; a start methionine is included. | | | |
| GTF 6855, Streptococcus salivarius SK126. The first 178 | 3 ^{a} | 4 | (1341 aa) |
| amino acids of the protein are deleted compared to | | | |
| GENBANK Identification No. 228476855 (Acc. No. | | | |
| ZP_04061500.1); a start methionine is included. | | | |
| GTF 2379, Streptococcus salivarius. The first 203 amino | 5 ^{a} | 6 | (1247 aa) |
| acids of the protein are deleted compared to GENBANK | | | |
| Identification No. 662379; a start methionine is included. | | | |
| GTF 7527 or GTFJ, Streptococcus salivarius. The first 42 | 7 ^{a} | 8 | (1477 aa) |
| amino acids of the protein are deleted compared to | | | |
| GENBANK Identification No. 47527; a start methionine is | | | |
| included. | | | |
| GTF 1724, Streptococcus downei. The first 162 amino acids | 9 ^{a} | 10 | (1436 aa) |
| of the protein are deleted compared to GENBANK | | | |
| Identification No. 121724; a start methionine is included. | | | |
| GTF 0544, Streptococcus mutans. The first 164 amino acids | 11 ^{a} | 12 | (1313 aa) |
| of the protein are deleted compared to GENBANK | | | |
| Identification No. 290580544; a start methionine is included. | | | |
| GTF 5926, Streptococcus dentirousetti. The first 144 amino | 13 ^{a} | 14 | (1323 aa) |
| acids of the protein are deleted compared to GENBANK | | | |
| Identification No. 167735926; a start methionine is included. | | | |
| GTF 4297, Streptococcus oralis. The first 228 amino acids of | 15 ^{a} | 16 | (1348 aa) |
| the protein are deleted compared to GENBANK Identification | | | |
| No. 7684297; a start methionine is included. | | | |
| GTF 5618, Streptococcus sanguinis. The first 223 amino | 17 ^{a} | 18 | (1348 aa) |
| acids of the protein are deleted compared to GENBANK | | | |
| Identification No. 328945618; a start methionine is included. | | | |
| GTF 2765, unknown Streptococcus sp. C150. The first 193 | 19 ^{a} | 20 | (1340 aa) |
| amino acids of the protein are deleted compared to | | | |
| GENBANK Identification No. 322372765; a start methionine is | | | |
| included. | | | |
| GTF 0427, Streptococcus sobrinus. The first 156 amino acids | 25 ^{a} | 26 | (1435 aa) |
| of the protein are deleted compared to GENBANK | | | |
| Identification No. 940427; a start methionine is included. | | | |
| GTF 2919, Streptococcus salivarius PS4. The first 92 amino | 27 ^{a} | 28 | (1340 aa) |
| acids of the protein are deleted compared to GENBANK | | | |
| Identification No. 383282919; a start methionine is included. | | | |
| GTF 2678, Streptococcus salivarius K12. The first 188 amino | 29 ^{a} | 30 | (1341 aa) |
| acids of the protein are deleted compared to GENBANK | | | |
| Identification No. 400182678; a start methionine is included. | | | |
| GTF 3929, Streptococcus salivarius JIM8777. The first 178 | 33 ^{a} | 34 | (1341 aa) |
| amino acids of the protein are deleted compared to | | | |
| GENBANK Identification No. 387783929; a start methionine is | | | |
| included. | | | |
| GTF 3298, Streptococcus sp. C150. The first 209 amino | | 59 | (1242 aa) |
| acids of the protein are deleted compared to GENBANK | | | |
| Identification No. 322373298; a start methionine is included. | | | |
| Wild type GTFJ, Streptococcus salivarius. GENBANK | | 60 | (1518 aa) |
| Identification No. 47527. | | | |
| Wild type GTF corresponding to GTF 2678, Streptococcus | | 61 | (1528 aa) |
| salivarius K12. | | | |
| Wild type GTF corresponding to GTF 6855, Streptococcus | | 62 | (1518 aa) |
| salivarius SK126. | | | |
| Wild type GTF corresponding to GTF 2919, Streptococcus | | 63 | (1431 aa) |
| salivarius PS4. | | | |
| Wild type GTF corresponding to GTF 2765, unknown | | 64 | (1532 aa) |
| Streptococcus sp. C150. | | | |
| Shorter version of GTF 7527, Streptococcus salivarius, (also | | 65 | (1341 aa) |
| referred to as “7527-NT” herein. The first 178 amino acids of | | | |
| the protein are deleted compared to GENBANK Identification | | | |
| No. 47527; a start methionine is included. | | | |
| Catalytic domain (approx.) of GTF 6855 (i.e., positions 55-960 | | 66 | (906 aa) |
| of SEQ ID NO: 4), but having a Glu at position 279, which | | | |
| corresponds to position 510 (Ala) of SEQ ID NO: 62. | | | |
| Terminator sequence added to pHY300PLK to derive the | 67 | | |
| pHYT vector. | | | |
| Catalytic domain (approx.) of GTF 6855 (i.e., positions 55-960 | | 71 | (906 aa) |
| of SEQ ID NO: 4), but having an Asp at position 279, which | | | |
| corresponds to position 510 (Ala) of SEQ ID NO: 62. | | | |

^{a} This DNA coding sequence is codon-optimized for expression in E. coli, and is merely disclosed as an example of a suitable coding sequence.
^{b}SEQ ID NOs: 21-24, 31, 32, 35-58 and 68-70 are intentionally not included in this table and merely serve as placeholders.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include alpha-1,3-glucan.

The terms “poly alpha-1,3-glucan”, “alpha-1,3-glucan”, “alpha-1,3-glucan polymer” and the like are used interchangeably herein. Alpha-1,3-glucan is a polymer comprising glucose monomeric units linked together by glycosidic linkages, wherein at least about 30% of the glycosidic linkages are alpha-1,3. Alpha-1,3-glucan in certain embodiments comprises at least about 90% or 95% alpha-1,3 glycosidic linkages. Most or all of the other linkages in alpha-1,3-glucan herein typically are alpha-1,6, though some linkages may also be alpha-1,2 and/or alpha-1,4.

The terms “glycosidic linkage”, “glycosidic bond”, “linkage” and the like are used interchangeably herein and refer to the covalent bond that joins a carbohydrate (sugar) molecule to another group such as another carbohydrate. The term “alpha-1,3-glycosidic linkage” as used herein refers to the type of covalent bond that joins alpha-D-glucose molecules to each other through carbons 1 and 3 on adjacent alpha-D-glucose rings. The term “alpha-1,6-glycosidic linkage” as used herein refers to the covalent bond that joins alpha-D-glucose molecules to each other through carbons 1 and 6 on adjacent alpha-D-glucose rings. The glycosidic linkages of a glucan polymer herein can also be referred to as “glucosidic linkages”. Herein, “alpha-D-glucose” is referred to as “glucose”.

The glycosidic linkage profile of an alpha-glucan herein can be determined using any method known in the art. For example, a linkage profile can be determined using methods using nuclear magnetic resonance (NMR) spectroscopy (e.g., ¹³C NMR or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, Fla., 2005), which is incorporated herein by reference.

The “molecular weight” of large alpha-glucan polymers herein can be represented as weight-average molecular weight (Mw) or number-average molecular weight (Mn), the units of which are in Daltons or grams/mole. Alternatively, the molecular weight of large alpha-glucan polymers can be represented as DP_{w} (weight average degree of polymerization) or DPₙ (number average degree of polymerization). The molecular weight of smaller alpha-glucan polymers such as oligosaccharides typically can be provided as “DP” (degree of polymerization), which simply refers to the number of glucoses comprised within the alpha-glucan. Various means are known in the art for calculating these various molecular weight measurements such as with high-pressure liquid chromatography (HPLC), size exclusion chromatography (SEC), or gel permeation chromatography (GPC).

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein.

The terms “particle”, “primary particle” and the like are interchangeably used herein. A particle is the smallest identifiable unit in a particulate system using the technology employed in the below Examples (or similar technology), and is a subunit of an aggregate. Particles herein have an average size of about 5-25 nm (nanometers). Size in some aspects can refer to particle diameter and/or the length of the longest particle dimension (e.g., length of a rod-like particle). The average size can be based on the average of diameters and/or longest dimensions of at least 50, 100, 500, 1000, 2500, 5000, or 10000 or more particles, for example.

The terms “aggregate”, “particle aggregate”, “particle cluster” and the like are interchangeably used herein. An aggregate is a body or mass comprising particles, and typically is formed by the clustering/aggregation (coming together) of particles in water or aqueous solution. Aggregates herein can form, for example, following dispersal of the particles in water or aqueous solution. Aggregates can be comprised within an agglomerate. Aggregates herein have an average hydrodynamic radius (Rₕ) of about 50-300 nm. Size in some aspects can instead refer to aggregate hydrodynamic diameter (Dₕ), diameter and/or the length of the longest aggregate dimension. Any of the foregoing average measurements can be obtained with at least 50, 100, 500, 1000, 2500, 5000, or 10000 or more aggregates, for example.

The “hydrodynamic radius” (Rₕ) of an aggregate as presently disclosed can be measured by dynamic light scattering (DLS) following the methodology described in the below Examples and/or in U.S. Patent Appl. Publ. Nos. 2017/0055540 or 2010/0056361, for example, which are incorporated herein by reference. DLS can also be referred to as photon correlation spectroscopy (PCS) or quasi-elastic light scattering. The hydrodynamic radius of an aggregate is the radius of a hypothetical hard sphere that diffuses in the same fashion as the aggregate. Such a measurement is made since the disclosed aggregates are typically non-spherical and dynamic in motion (tumbling). The size of an aggregate can also be referred to in terms of hydrodynamic diameter (Dₕ), if desired, which is two times its Rₕ value.

The term “fractal dimension” as used herein describes the openness of an aggregate structure. To illustrate how the fractal dimension of an aggregate characterizes its openness, it is instructional to consider, for example, a 100-nm aggregate comprising 10-nm particles. If the fractal dimension of the aggregate in this example is 3, the density at the center of the aggregate will be the same as the density at the periphery of the aggregate. A fractal dimension of 3 describes a space-filling object and designates a closed structure in terms of internal surface area. If the fractal dimension of the aggregate is 2, the density at the aggregate's periphery will be ten times less than the density at its center. If the fractal dimension of the aggregate is 1, the density at the aggregate's periphery will be 100 times less than the density at its center. Aggregates of the present disclosure have open structures with high internal surface areas; their fractal dimension of 1.6-2.4 indicates a reduction in density from aggregate center to periphery. Average fractal dimension can be based on the average of the fractal dimensions of at least 50, 100, 500, 1000, 2500, 5000, or 10000 or more aggregates, for example.

The term “arborescent” and like terms can optionally be used herein to characterize the treelike/branching nature/structure of insoluble alpha-glucan aggregates.

The terms “rod-like”, “rod-shaped” and like terms can be used to characterize primary particles in some aspects. A particle that is rod-like has a cylindrical shape with a length that is typically greater (e.g., at least 10%) than its cross-sectional diameter. The term “agglomerate” as used herein refers to a cluster of aggregates.

Agglomerates herein have an average size of about 1-200 microns (micrometers). Size in some aspects can refer to agglomerate diameter and/or the length of the longest agglomerate dimension. The average size can be based on the average of diameters and/or longest dimensions of at least 50, 100, 500, 1000, 2500, 5000, or 10000 or more agglomerates, for example.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37:D233-238, 2009).

The term “glucosyltransferase catalytic domain” herein refers to the domain of a glucosyltransferase enzyme that provides alpha-glucan-synthesizing activity to a glucosyltransferase enzyme. A glucosyltransferase catalytic domain preferably does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components. Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher (e.g., DP100 and higher). It would be understood that certain glucan products, such as alpha-1,3-glucan with a degree of polymerization (DP) of at least 8 or 9, are water-insoluble and thus not dissolved in a glucan synthesis reaction, but rather may be present out of solution (e.g., by virtue of having precipitated from the reaction). It is in a glucan synthesis reaction where the step of contacting water, sucrose and a glucosyltransferase enzyme is performed. The term “under suitable reaction conditions” as used herein refers to reaction conditions that support conversion of sucrose to alpha-glucan product(s) via glucosyltransferase enzyme activity. It is during such a reaction that glucosyl groups originally derived from the input sucrose are enzymatically transferred and used in alpha-glucan polymer synthesis; glucosyl groups as involved in this process can thus optionally be referred to as the glucosyl component or moiety (or like terms) of a glucosyltransferase reaction.

The “yield” of insoluble alpha-glucan product in a glucosyltransferase reaction in some aspects herein represents the molar yield based on the converted sucrose. The molar yield of an alpha-glucan product can be calculated based on the moles of insoluble alpha-glucan product divided by the moles of the sucrose converted. Moles of converted sucrose can be calculated as follows: (mass of initial sucrose—mass of final sucrose)/molecular weight of sucrose [342 g/mol]. This molar yield calculation can be considered as a measure of selectivity of the reaction toward the alpha-glucan. In some aspects, the “yield” of insoluble alpha-glucan product in a glucosyltransferase reaction can be based on the glucosyl component of the reaction. Such a yield (yield based on glucosyl) can be measured using the following formula:

Insoluble Alpha-Glucan Yield=((IS/2-(FS/2+LE/2+GL+SO))/(IS/2-FS/2))×100%.

The fructose balance of a glucosyltransferase reaction can be measured to ensure that HPLC data, if applicable, are not out of range (90-110% is considered acceptable). Fructose balance can be measured using the following formula:

Fructose Balance=((180/342×(FS+LE)+FR)/(180/342×IS))×100%.

In the above two formulae, IS is [Initial Sucrose], FS is [Final Sucrose], LE is [Leucrose], GL is [Glucose], SO is [Soluble Oligomers] (gluco-oligosaccharides), and FR is [Fructose]; the concentrations of each foregoing substrate/product provided in double brackets are in units of grams/L and as measured by HPLC, for example.

A “cake” of insoluble alpha-glucan herein refers to a preparation in condensed, compacted, packed, squeezed, and/or compressed form that comprises at least (i) about 50%-90% by weight water or an aqueous solution, and (ii) about 10%-50% by weight insoluble alpha-glucan. A cake in some aspects can be referred to as a “filter cake” or a “wet cake”. A cake herein typically has a soft, solid-like consistency.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “aqueous liquid”, “aqueous fluid” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises at least about 10 wt % water, for example. Examples of aqueous compositions include mixtures, solutions, dispersions (e.g., colloidal dispersions), suspensions and emulsions, for example. An aqueous composition in certain embodiments can comprise aggregates of insoluble alpha-glucan as disclosed herein, in which case the aqueous composition can optionally be characterized as a solid-in-liquid composition, given the insolubility of the aggregates.

As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. All, or a portion of, the particles of a colloidal dispersion such as a hydrocolloid can comprise aggregates of the present disclosure. The terms “dispersant” and “dispersion agent” are used interchangeably herein to refer to a material that promotes the formation and/or stabilization of a dispersion.

A glucan that is “insoluble”, “aqueous-insoluble”, “water-insoluble” (and like terms) (e.g., insoluble alpha-1,3-glucan) does not dissolve (or does not appreciably dissolve) in water or other aqueous conditions, optionally where the aqueous conditions are further characterized to have a pH of 4-9 (e.g., pH 6-8) and/or temperature of about 1 to 85° C. (e.g., 20-25° C.). In contrast, glucans such as certain oligosaccharides herein that are “soluble”, “aqueous-soluble”, “water-soluble” and the like (e.g., alpha-1,3-glucan with a DP less than 8) appreciably dissolve under these conditions.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa·s), for example. A centipoise is one one-hundredth of a poise; one poise is equal to 0.100 kg·m⁻¹ s⁻¹.

The terms “sequence identity”, “identity” and the like as used herein with respect to a polypeptide amino acid sequence are as defined and determined in U.S. Pat. Appl. Publ. No. 2017/0002336, which is incorporated herein by reference.

Various polypeptide amino acid sequences and polynucleotide sequences are disclosed herein as features of certain embodiments. Variants of these sequences that are at least about 70-85%, 85-90%, or 90%-95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence. Any polypeptide amino acid sequence disclosed herein not beginning with a methionine can typically further comprise at least a start-methionine at the N-terminus of the amino acid sequence. In contrast, any polypeptide amino acid sequence disclosed herein beginning with a methionine can optionally lack such a methionine residue.

The terms “aligns with”, “corresponds with”, and the like can be used interchangeably herein. Some embodiments herein relate to a glucosyltransferase comprising at least one amino acid substitution at a position corresponding with at least one particular amino acid residue of SEQ ID NO:62. An amino acid position of a glucosyltransferase or subsequence thereof (e.g., catalytic domain or catalytic domain plus glucan-binding domains) (can refer to such an amino acid position or sequence as a “query” position or sequence) can be characterized to correspond with a particular amino acid residue of SEQ ID NO:62 (can refer to such an amino acid position or sequence as a “subject” position or sequence) if (1) the query sequence can be aligned with the subject sequence (e.g., where an alignment indicates that the query sequence and the subject sequence [or a subsequence of the subject sequence] are at least about 30%, 40%, 50%, 60%, 70%, 80% or 90% identical), and (2) if the query amino acid position directly aligns with (directly lines up against) the subject amino acid position in the alignment of (1). In general, one can align a query amino acid sequence with a subject sequence (SEQ ID NO:62 or a subsequence of SEQ ID NO:62) using any alignment algorithm, tool and/or software described disclosed herein (e.g., BLASTP, ClustalW, ClustalV, Clustal-Omega, EMBOSS) to determine percent identity. Just for further example, one can align a query sequence with a subject sequence herein using the Needleman-Wunsch algorithm (Needleman and Wunsch, J. Mol. Biol. 48:443-453, 1970) as implemented in the Needle program of the European Molecular Biology Open Software Suite (EMBOSS [e.g., version 5.0.0 or later], Rice et al., Trends Genet. 16:276-277, 2000). The parameters of such an EMBOSS alignment can comprise, for example: gap open penalty of 10, gap extension penalty of 0.5, EBLOSUM62 (EMBOSS version of BLOSUM62) substitution matrix.

The numbering of particular amino acid residues of SEQ ID NO:62 herein is with respect to the full-length amino acid sequence of SEQ ID NO:62. The first amino acid (i.e., position 1, Met-1) of SEQ ID NO:62 is at the start of the signal peptide. Unless otherwise disclosed, substitutions herein are with respect to the full-length amino acid sequence of SEQ ID NO:62.

A “non-native glucosyltransferase” herein (alternatively, “mutant”, “variant”, “modified” and like terms can likewise be used to describe such a glucosyltransferase) has at least one amino acid substitution at a position corresponding with a particular amino acid residue of SEQ ID NO:62. In most cases, such at least one amino acid substitution is in place of the amino acid residue(s) that normally (natively) occurs at the same position in the native counterpart (parent) of the non-native glucosyltransferase. The amino acid normally occurring at the relevant site in the native counterpart glucosyltransferase often is the same as (or conserved with) the particular amino acid residue of SEQ ID NO:62 for which the alignment is made. A non-native glucosyltransferase optionally can have other amino acid changes (mutations, deletions, and/or insertions) relative to its native counterpart sequence.

It may be instructive to illustrate a substitution/alignment herein. SEQ ID NO:12 (GTF 0544) is a truncated form of a Streptococcus sobrinus glucosyltransferase. It is noted that Leu-193 of SEQ ID NO:12 corresponds with Leu-373 of SEQ ID NO:62 (alignment not shown). If SEQ ID NO:12 is mutated at position 193 to substitute the Leu residue with a different residue (e.g., Gln), then it can be stated that the position 193-mutated version of SEQ ID NO:12 represents a non-native glucosyltransferase having an amino acid substitution at a position corresponding with Leu-373 of SEQ ID NO:62, for example.

The term “isolated” means a substance (or process) in a form or environment that does not occur in nature. A non-limiting example of an isolated substance includes any non-naturally occurring substance such as an aggregate or particle of insoluble alpha-glucan herein (as well as the enzymatic reactions and other processes used in preparation of any of these materials). It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

New forms of insoluble alpha-glucan are desired to enhance the economic value and performance characteristics of this material in various applications. Compositions comprising insoluble alpha-glucan aggregates with unique morphological features are presently disclosed to address this need.

Certain embodiments of the present disclosure concern a composition comprising aggregates of insoluble alpha-glucan, wherein the aggregates have an average hydrodynamic radius of about 50-300 nm (nanometers) and a fractal dimension of about 1.6-2.4, and wherein the insoluble alpha-glucan comprises alpha-1,3-glycosidic linkages. The fractal dimension range of the disclosed aggregates indicates that they have a high internal surface area, which in turn indicates that the disclosed aggregates are useful in applications that take advantage of high internal surface area, for example.

The average hydrodynamic radius of aggregates herein is about 50-300 nm. In some aspects, the average hydrodynamic radius of the aggregates is about 50, 60, 70, 80, 90, 95, 100, 105, 110, 115, 120, 130, 140, 150, 160, 170, 180, 190, 200, 210, 220, 230, 240, 250, 260, 270, 280, 290, 300, 50-300, 50-250, 50-200, 50-150, 60-140, 60-130, 60-120, 60-115, 60-110, 60-105, 60-100, 70-130, 70-120, 70-115 70-110, 70-105, 70-100, 80-130, 80-120, 80-115, 80-110, 80-105, 80-100, 90-130, 90-120, 90-115, 90-110, 90-105, 90-100, 100-130, 100-120, 100-115, 100-110, or 100-105 nm. Yet in some aspects, the average hydrodynamic radius of aggregates can be about, or up to about, 300, 350, 400, 450, or 500 (i.e., 600, 700, 800, 900, or 1000 nm, respectively, in hydrodynamic diameter) (or any range therebetween). Aggregate hydrodynamic radius can be measured by dynamic light scattering (DLS) following the methodology described in the below Examples and/or as described in U.S. Patent Appl. Publ. Nos. 2017/0055540 or 2010/0056361, for example, which are incorporated herein by reference. In some aspects, hydrodynamic radius of aggregates can be as measured after disrupting a dispersion of insoluble alpha-glucan (e.g., about 0.05-0.15, or 0.1 mg/mL) by application of a shear force (e.g., using a sonicator) of about, or at least about, 8, 9, 10, 11, or 12 kJ/kg in specific energy (e.g., for about 30-50, 35-45, or 40 minutes). This measurement can be made immediately (e.g., within 1-5 or 1-10 minutes), or within about 1, 3, 6, 12, 18, 24, 30, 36, 42, or 48 hours, after ending the shear force application, for example. Aggregate size in some additional or alternative aspects can be measured using any suitable technique known in the art, such as electron microscopy (transmission [TEM] or scanning [SEM]), atomic force microscopy (AFM), small angle X-ray scattering (SAXS), small angle neutron scattering (SANS), light scattering, and/or by employing the techniques disclosed in the below Examples.

Aggregates of insoluble alpha-glucan herein have a fractal dimension of about 1.6-2.4. In some aspects, fractal dimension can be about 1.6, 1.7, 1.8. 1.9, 1.95, 2.0, 2.05, 2.1, 2.2, 2.3, 2.4, 1.6-2.4, 1.7-2.4, 1.8-2.4, 1.9-2.4, 1.95-2.4, 2.0-2.4, 1.6-2.3, 1.7-2.3, 1.8-2.3, 1.9-2.3, 1.95-2.3, 2.0-2.3, 1.6-2.2, 1.7-2.2, 1.8-2.2, 1.9-2.2, 1.95-2.2, 2.0-2.2, 1.6-2.1, 1.7-2.1, 1.8-2.1, 1.9-2.1, 1.95-2.1, 2.0-2.1, 1.6-2.05, 1.7-2.05, 1.8-2.05, 1.9-2.05, 1.95-2.05, or 2.0-2.05. Fractal dimension can be measured using any suitable technique known in the art, such as scattering (light scattering, X-ray scattering [e.g., SAXS], or neutron scattering [e.g., SANS]) and/or by employing the techniques disclosed in the below Examples. Aggregates herein can optionally be characterized to be flat in shape/morphology; in general, the closer the fractal dimension is to 2.0 (as approached from above 2.0), the flatter the shape. Fractal dimension in some aspects can be measured with respect to aggregates that are about 10-150, 10-125, 10-110, or 10-100 nm in hydrodynamic radius; however, fractal dimension of aggregates of any hydrodynamic radius (or range thereof) as listed above (e.g., up to 500 nm) can also be measured, if desired.

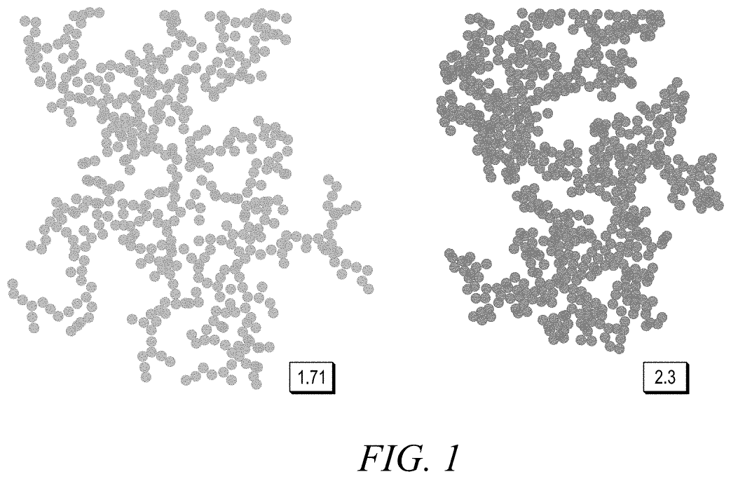

Aggregates in some aspects are arborescent, and thus have a treelike or branching structure. The arborescent nature of aggregates can be as depicted in FIG. 1, for example. This arborescence is contemplated to result from the fractal organization of insoluble alpha-glucan particles that form an aggregate. Aggregate arborescence herein can be detected by microscopy (e.g., transmission electron microscopy [TEM] such as cryogenic TEM [a.k.a. cryo-TEM]), for example, and/or by employing the techniques disclosed in the below Examples.

Aggregates herein have a high surface area. It is contemplated that the surface area of aggregates in some aspects is about, or at least about, 100, 125, 150, 175, 200, 225, 250, 275, 300, 150-250, 150-225, 175-250, or 175-225 m²/g. Surface area in some particular aspects characterizes aggregates comprising particles with an average size of about 13-17 nm (e.g., ˜15 nm).

Aggregates herein comprise particles (primary particles) of insoluble alpha-glucan. These particles can have an average size of about 5-25 nm, for example. In some aspects, particles of insoluble alpha-glucan can have an average size of about 5, 10, 15, 20, 25, 10-20, 10-18, 10-16, 12-20, 12-18, 12-16, 14-20, 14-18, or 14-16 nm. Particle size can be measured using any suitable technique known in the art (e.g., as described above for measuring aggregate size, such as SAXS), and/or by employing the techniques disclosed in the below Examples. Aggregates in some aspects consist of insoluble alpha-glucan particles. Particles can be spherical and/or rod-like in shape, for example. It is contemplated that, in some aspects, about 0%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the particles of aggregates herein are (i) spherical, (ii) rod-like, or a combination of (i) and (ii) adding up to about 80%, 85%, 90%, 95%, or 100%. In some aspects, primary particle size (e.g., diameter) can be as measured using small angle scattering (e.g., with data subjected to a universal fit, such as per Beaucage [1995, J. Appl. Cryst. 28:717-728, incorporated herein by reference]) to calculate particle radius of gyration (R_{g}), which is then used in the following equation to calculate particle diameter (D):

$$D = 2 \sqrt{\frac{5}{3}} {R}_{g} .$$

In any aqueous setting as presently disclosed, aggregates are generally recalcitrant to disruption (generally resistant to being torn apart) when subjected to a shear force with specific energy of up to several MJ/kg (e.g., contemplated to be up to about 3, 4, 5, 6, 7, or 8 MJ/kg in specific energy) (e.g., for up to 30-40 minutes); for example, it is contemplated that up to 80%, 85%, 90% or 95% of aggregates are not disrupted to primary particles when subjected to a shear force herein.

Individual molecules of insoluble alpha-glucan in a particle herein each can have a weight-average degree of polymerization (DPw) of about, or at least about, 100, 200, 300, 400, 500, 600, 700, 800, 900, 1000, 1100, 1200, 200-1200, 400-1200, 600-1200, 200-1000, 400-1000, or 600-1000, for example. There can be about 6, 7, 8, 9, 10, 11, 12, 6-12, or 8-12 individual molecules of insoluble alpha-glucan in a particle in some aspects; this molecule number typically depends on the individual molecule DPw and the size of the particle (e.g., a 14-16-nm particle can have 10 molecules that are each 800 DPw).

A composition in certain embodiments can comprise agglomerates of aggregates as presently disclosed. Agglomerates can have an average size of about 1, 5, 10, 25, 50, 75, 90, 100, 110, 125, 150, 200, 250, 300, 350, 400, 1-200, 2.5-200, 5-200, 7.5-200, 1-150, 2.5-150, 5-150, 7.5-150, 1-125, 2.5-125, 5-125, 7.5-125, 1-110, 2.5-110, 5-110, 7.5-110, 1-100, 2.5-100, 5-100, 7.5-100, 1-90, 2.5-90, 5-90, 7.5-90, 1-50, 5-50, 10-50, or 25-50 microns (micrometers), for example. In any aqueous setting as presently disclosed, agglomerates herein can typically be shorn/disrupted/torn apart by a shear force of about, or at least about, 8, 9, 10, 11, or 12 kJ/kg in specific energy (e.g., for about 3, 4, 5, 6, 8, or 10 minutes), for example, to produce smaller agglomerates or constituent aggregates. In turn, aggregates can reassemble into agglomerates upon removal of such a shear force. In some aspects, the agglomerate size values disclosed herein represent the median diameter (D50) of a particle size distribution as measured using a suitable particle size analyzer (e.g., based on light scattering) such as a Mastersizer® (Malvern) and/or as described in the below Examples. Agglomerate size in some aspects is as measured for non-disrupted (e.g., non-sonicated) agglomerates.

Aggregates of the present disclosure comprise insoluble alpha-glucan comprising alpha-1,3-glycosidic linkages. In some aspects, at least about 50%, 60%, 70%, 80%, 85%, 90%, 91%, 92%, 93%, 94%, 95%, 96%, 97%, 98%, 99%, 99.5%, or 100% (or any integer between 50% and 100%) of the constituent glycosidic linkages of insoluble alpha-glucan are alpha-1,3 linkages. In some aspects, accordingly, insoluble alpha-glucan has less than about 50%, 40%, 30%, 20%, 15%, 10%, 9%, 8%, 7%, 6%, 5%, 4%, 3%, 2%, 1%, 0.5%, or 0% (or any integer value between 0% and 50%) glycosidic linkages that are not alpha-1,3. Typically, the glycosidic linkages that are not alpha-1,3 are mostly or entirely alpha-1,6. It should be understood that the higher the percentage of alpha-1,3 linkages present in alpha-glucan, the greater the probability that the alpha-glucan is linear, since there are lower occurrences of certain linkages forming branch points in the polymer. Thus, insoluble alpha-glucan with 100% alpha-1,3 linkages is believed to be completely linear. In certain embodiments, insoluble alpha-glucan has no branch points or less than about 5%, 4%, 3%, 2% or 1% branch points as a percent of the glycosidic linkages in the polymer. Examples of branch points include alpha-1,6, -1,2 and -1,4 branch points stemming from an alpha-1,3-linked backbone.

Insoluble alpha-glucan herein can have a molecular weight in DPw or DPn of about, or at least about, 100 in some aspects. DPw or DPn in some embodiments can be about, or at least about, 100, 150, 200, 250, 300, 350, 400, 450, 500, 550, 600, 650, 700, 750, 800, 850, 900, 950, 1000, 1100, or 1200 (or any integer between 100 and 1200). The DPw or DPn of an alpha-glucan can optionally be expressed as a range between any two of these values (e.g., 100-1200, 400-1200, 700-1200, 100-1000, 400-1000, 700-1000).

Alpha-glucan herein is insoluble in non-caustic aqueous systems, such as those conditions of a glucosyltransferase reaction herein (e.g., pH 4-8, see below). In general, the solubility of a glucan polymer in aqueous settings herein is related to its linkage profile, molecular weight, and/or degree of branching. For example, alpha-1,3-glucan with ≥95%, 1,3 linkages is generally insoluble at a DPw of 8 and above in aqueous conditions at 20° C. In general, as molecular weight increases, the percentage of alpha-1,3 linkages required for alpha-1,3-glucan insolubility decreases.

In some embodiments, an insoluble alpha-glucan can comprise at least about 30% alpha-1,3 linkages and a percentage of alpha-1,6 linkages that brings the total of both the alpha-1,3 and -1,6 linkages in the alpha-glucan to 100%. For example, the percentage of alpha-1,3 and -1,6 linkages can be about 30-40% and 60-70%, respectively. In some aspects, an insoluble alpha-glucan comprising at least about 30% alpha-1,3 linkages is linear. Glucosyltransferases for producing insoluble alpha-glucan comprising at least about 30% alpha-1,3 linkages are disclosed in U.S. Pat. Appl. Publ. No. 2015/0232819, which is incorporated herein by reference.

Insoluble alpha-glucan in some embodiments can be in the form of a copolymer (e.g., graft copolymer) having (i) a backbone comprising dextran (e.g., with at least about 95%, 96%, 97%, 98%, 99%, or 100% alpha-1,6 linkages) with a molecular weight of at least about 100000 Daltons, and (ii) alpha-1,3-glucan side chains comprising at least about 95%, 96%, 97%, 98%, 99%, or 100% alpha-1,3-glucosidic linkages. Such copolymers can be as disclosed in Int. Pat. Appl. Publ. No. WO2017/079595, which is incorporated herein by reference.

Any of the foregoing linkage profiles and/or molecular weight profiles, for example, can be combined herein to appropriately characterize insoluble alpha-glucan herein. In some aspects, the linkage and/or molecular weight profile of such alpha-glucan can be as disclosed in any of the following publications, all of which are incorporated herein by reference: U.S. Pat. Nos. 7,000,000 and 8,871,474, U.S. Patent Appl. Publ. No. 2015/0232819, Int. Pat. Appl. Publ. No. WO2017/079595. Insoluble alpha-glucan of the foregoing embodiments can be a product of any of the glucan synthesis reaction and post-reaction processes disclosed below, for example.

Insoluble alpha-glucan herein does not comprise alternan (alternating 1,3 and 1,6 linkages), which is aqueous-soluble. Insoluble alpha-glucan herein is typically enzymatically derived in an inert vessel (typically under cell-free conditions), and is not derived from a cell wall (e.g., fungal cell wall).

Certain embodiments of the present disclosure concern a method of producing (preparing) aggregates of insoluble-alpha-glucan as described herein. Such a method comprises: (a) contacting at least water, sucrose, and a glucosyltransferase enzyme that synthesizes insoluble alpha-glucan at a yield of at least about 40%; and (b) preparing a dispersion of the insoluble alpha-glucan produced in step (a). Step (a) can optionally be characterized to comprise preparing/providing a glucosyltransferase reaction or reaction composition. Step (a) can employ a glucosyltransferase enzyme that produces any insoluble alpha-glucan molecule as disclosed above (e.g., ≥90% or ≥95% alpha-1,3-linkages). Dispersion step (b) produces aggregates of the insoluble alpha-glucan synthesized in step (a).

A glucosyltransferase reaction as presently disclosed comprises contacting at least water, sucrose, and a glucosyltransferase herein that produces insoluble alpha-glucan. These and optionally other reagents can be added altogether or in any order as discussed below. The contacting step herein can be performed in any number of ways. For example, the desired amount of sucrose can first be dissolved in water (optionally, other components may also be added at this stage of preparation, such as buffer components), followed by addition of glucosyltransferase enzyme. The solution may be kept still, or agitated via stirring or orbital shaking, for example. A glucosyltransferase reaction can be performed by batch, fed-batch, continuous mode, or by any variation of these modes.

Completion of a reaction in certain embodiments can be determined visually (e.g., no more accumulation of insoluble alpha-glucan), and/or by measuring the amount of sucrose left in the solution (residual sucrose), where a percent sucrose consumption of at least about 90%, 95%, or 99% can indicate reaction completion. In some aspects, a reaction can be considered complete when its sucrose content is at or below about 2-5 g/L. A reaction of the disclosed process can be conducted for about 1 hour to about 2, 4, 6, 8, 10, 12, 14, 16, 18, 20, 22, 24, 36, 48, 60, 72, 96, 120, 144, or 168 hours, for example. A reaction can optionally be terminated and/or otherwise treated to stop glucosyltransferase activity, e.g., by heating it to at least about 65° C. for at least about 30-60 minutes.

The temperature of a reaction composition herein can be controlled, if desired, and can be about 5-50° C., 20-40° C., 30-40° C., 20-30° C., 20-25° C., 20° C., 25° C., 30° C., 35° C., or 40° C., for example.

The initial concentration of sucrose in a reaction composition herein can be about 20-400 g/L, 75-175 g/L, or 50-150 g/L, for example. In some aspects, the initial sucrose concentration is at least about 50, 75, 100, 150 or 200 g/L, or is about 50-600 g/L, 100-500 g/L, 50-100 g/L, 100-200 g/L, 150-450 g/L, 200-450 g/L, or 250-600 g/L. “Initial concentration of sucrose” refers to the sucrose concentration in a reaction composition just after all the reaction components have been added/combined (e.g., at least water, sucrose, glucosyltransferase enzyme).

The pH of a reaction composition in certain embodiments can be about 4.0-9.0, 4.0-8.5, 4.0-8.0, 5.0-8.0, 5.5-7.5, or 5.5-6.5. In some aspects, the pH can be about 4.0, 4.5, 5.0, 5.5, 6.0, 6.5, 7.0, 7.5, or 8.0. The pH can be adjusted or controlled by the addition or incorporation of a suitable buffer, including but not limited to: phosphate, tris, citrate, or a combination thereof. The buffer concentration in a reaction composition herein can be about 0.1-300 mM, 0.1-100 mM, 10-100 mM, 10 mM, 20 mM, or 50 mM, for example.

A glucosyltransferase reaction can be contained within any vessel (e.g., an inert vessel/container) suitable for applying one or more of the reaction conditions disclosed herein. An inert vessel in some aspects can be of stainless steel, plastic, or glass (or comprise two or more of these components) and be of a size suitable to contain a particular reaction. For example, the volume/capacity of an inert vessel (and/or the volume of a reaction composition herein) can be about, or at least about, 1, 10, 50, 100, 500, 1000, 2500, 5000, 10000, 12500, 15000, or 20000 liters. An inert vessel can optionally be equipped with a stirring device. Any of the foregoing features, for example, can be used to characterize an isolated reaction herein.

A reaction composition herein can contain one, two, or more different glucosyltransferase enzymes, for example. In some embodiments, only one or two glucosyltransferase enzymes is/are comprised in a reaction composition. A glucosyltransferase reaction herein can be, and typically is, cell-free (e.g., no whole cells present).

Examples of other conditions and/or components suitable for synthesizing insoluble alpha-glucan herein are disclosed in U.S. Patent Appl. Publ. Nos. 2014/0087431, 2017/0166938 and 2017/0002335, which are incorporated herein by reference.

A glucosyltransferase enzyme herein synthesizes insoluble alpha-glucan at a yield of at least about 40%. The yield of insoluble alpha-glucan by a glucosyltransferase enzyme in some aspects can be about, or at least about, 40%, 45%, 50%, 55%, 60%, 65%, 70%, 75%, 80%, 85%, 90%, 91%, 92%, 93%, 94%, 95%, or 96%. Yield in some aspects can be measured based on the glucosyl component of the reaction. Step (a) can thus optionally be characterized to use a glucosyltransferase enzyme that synthesizes insoluble alpha-glucan at a yield that is at least about 40% based on the glucosyl component of the reaction composition. Yield in some aspects can be measured using HPLC or NIR spectroscopy. Yield can be achieved in a reaction conducted for about 16-24 hours (e.g., ˜20 hours), for example.

Examples of glucosyltransferase enzymes herein that synthesize insoluble alpha-glucan at a yield of at least about 40% include certain non-native glucosyltransferases as described below. A non-native glucosyltransferase, for instance, can (i) comprise or consist of an amino acid sequence that is at least about 90%, 91%, 92%, 93%, 94%, 95%, 96%, 97%, 98%, 99%, or 99.5% A identical to SEQ ID NO:2, 4, 6, 8, 10, 12, 14, 16, 18, 20, 26, 28, 30, 34, or 59; and (ii) have at least one amino acid substitution that provides the non-native glucosyltransferase the ability to produce insoluble alpha-glucan at a yield of at least 40% (examples of suitable substitutions are described below). Certain information regarding insoluble alpha-glucan products of glucosyltransferases with the foregoing amino acid sequences is provided in Table 2 (below). A non-native glucosyltransferase herein typically has a parent counterpart (e.g., native and/or unsubstituted) that has an insoluble alpha-glucan yield of less than about 33%, 32%, 31%, 30%, 29%, 28%, or 27%, for example.

TABLE 2
GTF Enzymes and Related Alpha-Glucan Products^{a}

| | | | | Linkages | | |
|---|---|---|---|---|---|---|
| | SEQ ID | Reducing | Insoluble | % alpha- | % alpha- | |
| GTF ID | NO. | Sugars | Product | 1,3 | 1,6 | DPₙ |
| 0874 | 2 | yes | yes | 100 | 0 | 60 |
| 6855 | 4 | yes | yes | 100 | 0 | 440 |
| 2379 | 6 | yes | yes | 37 | 63 | 310 |
| 7527 | 8 | yes | yes | 100 | 0 | 440 |
| 1724 | 10 | yes | yes | 100 | 0 | 250 |
| 0544 | 12 | yes | yes | 62 | 36 | 980 |
| 5926 | 14 | yes | yes | 100 | 0 | 260 |
| 4297 | 16 | yes | yes | 31 | 67 | 800 |
| 5618 | 18 | yes | yes | 34 | 66 | 1020 |
| 2765 | 20 | yes | yes | 100 | 0 | 280 |
| 0427 | 26 | yes | yes | 100 | 0 | 120 |
| 2919 | 28 | yes | yes | 100 | 0 | 250 |
| 2678 | 30 | yes | yes | 100 | 0 | 390 |
| 3929 | 34 | yes | yes | 100 | 0 | 280 |

^{a}GTF reactions and product analyses were performed as follows. Reactions were prepared comprising sucrose (50 g/L), potassium phosphate buffer (pH 6.5, 20 mM) and a GTF enzyme (2.5% bacterial cell extract by volume; extracts prepared according to U.S. application Pub. No. 2017/0002335, in a manner similar to procedure disclosed in U.S. Pat. No. 8,871,474). After 24-30 hours at 22-25° C., insoluble product was harvested by centrifugation, washed three times with water, washed once with ethanol, and dried at 50° C. for 24-30 hours.
Approximate linkages and DPₙ are shown for each insoluble product. Linkages and DPₙ were determined by ¹³C NMR and SEC, respectively.

In some aspects, a non-native glucosyltransferase comprises at least one amino acid substitution corresponding with a substitution in Table 3 (below, Example 1) that is associated with an insoluble alpha-glucan yield of at least 40% (the position numbering of such at least one substitution corresponds with the position numbering of SEQ ID NO:62). In some aspects, a non-native glucosyltransferase comprises a set of amino acid substitutions corresponding with a set of substitutions in Tables 4 or 5 (below, Example 1) that is associated with an insoluble alpha-glucan yield of at least 40% (the position numbering of this set of substitutions corresponds with the position numbering of SEQ ID NO:62).

In some aspects, a non-native glucosyltransferase (i) comprises at least one amino acid substitution or a set of amino acid substitutions (described above), and (ii) comprises or consists of a glucosyltransferase catalytic domain that is at least about 90%, 91%, 92%, 93%, 94%, 95%, 96%, 97%, 98%, 99%, or 99.5% identical to amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20. Such a non-native glucosyltransferase, for instance, is believed to be able to produce alpha-glucan that is water-insoluble and comprise at least about 50% (e.g., ≥90%, or ≥95%) alpha-1,3 linkages, and optionally further have a DP_{w} of at least 100. It is noted that a glucosyltransferase with amino acid positions 54-957 of SEQ ID NO:65 can produce alpha-1,3-glucan with 100% alpha-1,3 linkages and a DP_{w} of at least 400 (data not shown, refer to Table 6 of U.S. Pat. Appl. Publ. No. 2017/0002335, which is incorporated herein by reference), for example. It is further noted that SEQ ID NOs:65 (GTF 7527), 30 (GTF 2678), 4 (GTF 6855), 28 (GTF 2919), and 20 (GTF 2765) each represent a glucosyltransferase that, compared to its respective wild type counterpart, lacks the signal peptide domain and all or a substantial portion of the variable domain. Thus, each of these glucosyltransferase enzymes has a catalytic domain followed by a glucan-binding domain. The approximate location of catalytic domain sequences in these enzymes is as follows: 7527 (residues 54-957 of SEQ ID NO:65), 2678 (residues 55-960 of SEQ ID NO:30), 6855 (residues 55-960 of SEQ ID NO:4), 2919 (residues 55-960 of SEQ ID NO:28), 2765 (residues 55-960 of SEQ ID NO:20). The amino acid sequences of the catalytic domains (approx.) of GTFs 2678, 6855, 2919 and 2765 have about 94.9%, 99.0%, 95.5% and 96.4% identity, respectively, with the approximate catalytic domain sequence of GTF 7527 (i.e., amino acids 54-957 of SEQ ID NO:65). Each of these particular glucosyltransferases (GTFs 2678, 6855, 2919 and 2765) can produce alpha-1,3-glucan with 100% alpha-1,3 linkages and a DP_{w} of at least 400 (data not shown, refer to Table 4 of U.S. Pat. Appl. Publ. No. 2017/0002335). Based on this activity, and the relatedness (high percent identity) of the foregoing catalytic domains, it is contemplated that a non-native glucosyltransferase herein having one of the foregoing catalytic domains further with at least one amino acid substitution as presently disclosed can produce alpha-glucan comprising at least about 50% (e.g., ≥90% or ≥95%) alpha-1,3 linkages and a DP_{w} of at least 100.

In some aspects, a non-native glucosyltransferase (i) comprises at least one amino acid substitution or a set of amino acid substitutions (described above), and (ii) comprises or consists of an amino acid sequence that is at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 69%, 70%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to SEQ ID NO:62 or a subsequence thereof such as SEQ ID NO:4 (without start methionine thereof) or positions 55-960 of SEQ ID NO:4 (approximate catalytic domain).

A non-native glucosyltransferase enzyme herein can be derived from a microbial source such as bacteria. Examples of bacterial glucosyltransferase enzymes are those derived from a Streptococcus species, Leuconostoc species or Lactobacillus species. Examples of Streptococcus species include S. salivarius, S. sobrinus, S. dentirousetti, S. downei, S. mutans, S. oralis, S. gallolyticus and S. sanguinis. Examples of Leuconostoc species include L. mesenteroides, L. amelibiosum, L. argentinum, L. carnosum, L. citreum, L. cremoris, L. dextranicum and L. fructosum. Examples of Lactobacillus species include L. acidophilus, L. delbrueckii, L. helveticus, L. salivarius, L. casei, L. curvatus, L. plantarum, L. sakei, L. brevis, L. buchneri, L. fermentum and L. reuteri.

Step (b) of an aggregate production method herein comprises preparing a dispersion of the insoluble alpha-glucan produced in step (a). This dispersion can be a colloidal dispersion, for instance. Insoluble alpha-glucan produced by a glucosyltransferase reaction of step (a) can be removed from the reaction, for example, and then dispersed in water or an aqueous solution using any suitable method. In some aspects, such dispersal can be performed by applying high shear using any suitable means. High shear can be of about, or at least about, 8, 9, 10, 11, or 12 kJ/kg in specific energy, and/or can comprise mixing at about, or up to about, 3000, 4000, 6000, 8000, 10000, 12000, 14000, or 15000 rpm, for example. High shear can be applied for about 3, 4, 5, 6, 8, or 10 minutes, for example. Suitable means for applying high shear include, for example, using a suitable dispersal tool such as a disperser, sonicator (e.g., ultrasonicator), homomixer, homogenizer (e.g., rotary or piston, rotar-stator), microfluidizer, planetary mixer, colloid mill, jet mill, vortex, and/or any methodology as described in the below Examples and/or in International Patent Appl. Publ. Nos. WO2016/126685 or WO2016/030234, U.S. Pat. Nos. 5,767,176, 6,139,875, or 8722092, or U.S. Patent Appl. Publ. Nos. 2017/0055540 or 2018/0021238, all of which publications are incorporated herein by reference.

Insoluble alpha-glucan produced in step (a) of the presently disclosed method is typically isolated before dispersing the insoluble alpha-glucan in step (b) to obtain aggregates. In certain embodiments, isolating insoluble alpha-glucan can include at least conducting a step of centrifugation and/or filtration. Isolation can optionally further comprise washing insoluble alpha-glucan one, two, or more times with water or other aqueous liquid (e.g., using an aqueous solution herein). A wash volume can optionally be at least about 10-100% of the volume of the glucosyltransferase reaction used to produce the insoluble alpha-glucan, for example. Washing can be done by various modes, as desired, such as by displacement or re-slurry washing. Isolation herein does not comprise drying insoluble alpha-glucan.

Preparing a dispersion of insoluble alpha-glucan in some aspects can comprise: preparing a wet cake of insoluble alpha-glucan produced in the glucosyltransferase reaction of step (a), and dispersing the wet cake in water or an aqueous solution. Isolating insoluble alpha-glucan for wet cake preparation can include at least conducting a step of centrifugation (i.e., wet cake is pelleted glucan) and/or filtration (i.e., wet cake is filtered glucan). For example, wet cake herein can be obtained using a funnel, filter (e.g., a surface filter such as a rotary vacuum-drum filter, cross-flow filter, screen filter, belt filter, screw press, or filter press with or with membrane squeeze capability; or a depth filter such as a sand filter), and/or centrifuge; filtration can be by gravity, vacuum, or press filtration, for instance. Wet cake isolation can optionally further comprise washing it as described above. A wet cake herein can comprise, for example, at least (i) about 50%-90% by weight water or an aqueous solution, and (ii) about 10%-50% by weight insoluble alpha-glucan. A wet cake in some aspects can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 10-50, 10-40, 10-30, 10-20, 20-50, 20-40, 20-30, 30-50, 30-40, 40-50, 30-45, 35-45, 37.5-42.5, 35-40, or 40-45 wt % insoluble alpha-glucan, for example (with water or aqueous solution adding up to 100 wt %). In some aspects, the aqueous portion of a wet cake has a solute and/or pH profile according to that as described for an aqueous solution herein.

A composition of the present disclosure can comprise aggregates of insoluble alpha-1,3-glucan as produced in a method as described above, for example.

Aggregates of insoluble alpha-glucan herein can be present in a composition, such as an aqueous composition (e.g., colloidal dispersion), at a wt % of about, or at least about, 0.01, 0.05, 0.1, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt %, for example. The liquid component of an aqueous composition can be water or an aqueous solution, for instance. The solvent of an aqueous solution typically is water, or can comprise about, or at least about, 10, 20, 30, 40, 50, 60, 70, 80, 90, 95, or 98 wt % water, for example.

An aqueous solution in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. An aqueous solution in some aspects can have one or more salts/buffers (e.g., Na⁺, Cl⁻, NaCl, phosphate, tris, citrate) (e.g., ≤0.1, 0.5, 1.0, 2.0, or 3.0 wt %) and/or a pH of about 4.0, 4.5, 5.0, 5.5, 6.0, 6.5, 7.0, 7.5, 8.0, 8.5, 9.0, 4.0-9.0, 4.0-8.5, 4.0-8.0, 5.0-9.0, 5.0-8.5, 5.0-8.0, 6.0-9.0, 6.0-8.5, or 6.0-8.0, for example.

A composition comprising aggregates of the present disclosure can have a viscosity of about, or at least about, 10, 25, 50, 100, 250, 500, 750, 1000, 2000, 3000, 4000, 5000, 6000, 7000, 8000, 9000, 10000, 11000, 12000, 13000, 14000, 15000, 20000, 30000, 40000, 50000, 60000, 70000, 80000, 90000, 100000, 250000, 500000, or 1000000 centipoise (cP), for example. Viscosity can be measured using a viscometer or rheometer, or using any other means known in the art. A rotational shear rate of about, or at least about, 10, 60, 150, 250, 600, 800, 1000, or 10-1000 rpm (revolutions per minute), for example, can be applied when measuring the viscosity of a composition herein. Viscosity can be measured at any suitable temperature (e.g., between 4-30, 20-30, 20-25, or 3-110° C.), and/or at any suitable pressure (e.g., atmospheric pressure, about 760 torr).

A composition comprising aggregates of the present disclosure can, in some aspects, be non-aqueous (e.g., a dry composition). Examples of such embodiments include powders, granules, microcapsules, flakes, or any other form of particulate matter. Other examples include larger compositions such as pellets, bars, kernels, beads, tablets, sticks, or other agglomerates. A non-aqueous or dry composition typically has less than 3, 2, 1, 0.5, or 0.1 wt % water comprised therein.

A composition comprising aggregates herein, such as an aqueous composition or a non-aqueous composition (above), can be in the form of a household care product, personal care product, industrial product, pharmaceutical product, or food product, for example, such as described in any of U.S. Patent Appl. Publ. Nos. 2016/0311935, 2016/0304629, 2015/0232785, 2015/0368594, 2015/0368595 and 2016/0122445, and International Patent Appl. Publ. Nos. WO2016/160737, WO2016/160738, WO2016/133734 and WO2016/160740, which are all incorporated herein by reference. In some aspects, a composition comprising aggregates can comprise at least one component/ingredient of a household care product, personal care product, industrial product, pharmaceutical product, or food product as disclosed in any of the foregoing publications.

A composition comprising aggregates herein optionally can contain one or more active enzymes. Non-limiting examples of suitable enzymes include proteases, cellulases, hem icellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase, phenoloxidase), lipoxygenases, ligninases, pullulanases, tannases, pentosanases, malanases, beta-glucanases, arabinosidases, hyaluronidases, chondroitinases, laccases, metalloproteinases, amadoriases, galactosidases, galactanases, catalases, carageenases, lactases, phosphatases, polygalacturonases, rhamnogalactouronases, transglutaminases, xyloglucanases, xylosidases, metalloproteases, arabinofuranosidases, phytases, isomerases, transferases, glucosyltransferases (e.g., as disclosed herein), amylases (e.g., glucoamylases, alpha-amylases, beta-amylases), and any combination thereof. If an enzyme(s) is included, it may be comprised in a composition herein at about 0.0001-0.1 wt % (e.g., 0.01-0.03 wt %) active enzyme (e.g., calculated as pure enzyme protein), for example.

At least one, two, or more cellulases may be included in a composition herein. A cellulase herein can have endocellulase activity (EC 3.2.1.4), exocellulase activity (EC 3.2.1.91), or cellobiase activity (EC 3.2.1.21). A cellulase herein is an “active cellulase” having activity under suitable conditions for maintaining cellulase activity; it is within the skill of the art to determine such suitable conditions.

A cellulase herein may be derived from any microbial source, such as a bacteria or fungus. Chemically-modified cellulases or protein-engineered mutant cellulases are included. Suitable cellulases include, but are not limited to, cellulases from the genera Bacillus, Pseudomonas, Streptomyces, Trichoderma, Humicola, Fusarium, Thielavia and Acremonium. As other examples, a cellulase may be derived from Humicola insolens, Myceliophthora thermophila or Fusarium oxysporum; these and other cellulases are disclosed in U.S. Pat. Nos. 4,435,307, 5,648,263, 5,691,178, 5,776,757 and 7,604,974, which are all incorporated herein by reference. Exemplary Trichoderma reesei cellulases are disclosed in U.S. Pat. Nos. 4,689,297, 5,814,501, 5,324,649, and International Patent Appl. Publ. Nos. WO92/06221 and WO92/06165, all of which are incorporated herein by reference. Exemplary Bacillus cellulases are disclosed in U.S. Pat. No. 6,562,612, which is incorporated herein by reference. A cellulase, such as any of the foregoing, preferably is in a mature form lacking an N-terminal signal peptide. Commercially available cellulases useful herein include CELLUZYME® and CAREZYME® (Novozymes A/S); CLAZINASE® and PURADAX® HA (DuPont Industrial Biosciences), and KAC-500(B)® (Kao Corporation).

One or more cellulases can be directly added as an ingredient when preparing a composition disclosed herein. Alternatively, one or more cellulases can be indirectly (inadvertently) provided in the disclosed composition. For example, cellulase can be provided in a composition herein by virtue of being present in a non-cellulase enzyme preparation used for preparing a composition. Cellulase in compositions in which cellulase is indirectly provided thereto can be present at about 0.1-10 ppb (e.g., less than 1 ppm), for example.

The effective concentration of cellulase in an aqueous composition in which a fabric is treated can be readily determined by a skilled artisan. In fabric care processes, cellulase can be present in an aqueous composition (e.g., wash liquor) in which a fabric is treated in a concentration that is minimally about 0.01-0.1 ppm total cellulase protein, or about 0.1-10 ppb total cellulase protein (e.g., less than 1 ppm), to maximally about 100, 200, 500, 1000, 2000, 3000, 4000, or 5000 ppm total cellulase protein, for example.

Non-limiting examples of compositions and methods disclosed herein include:

1. A composition comprising aggregates of insoluble alpha-glucan, wherein the aggregates have an average hydrodynamic radius of about 50-300 nm and a fractal dimension of about 1.6-2.4, and the insoluble alpha-glucan comprises alpha-1,3-glycosidic linkages.
2. The composition of embodiment 1, wherein the aggregates are arborescent.
3. The composition of embodiment 1 or 2, wherein the aggregates have an average hydrodynamic radius of about 50-150 nm.
4. The composition of embodiment 1, 2, or 3, wherein the aggregates have an average hydrodynamic radius of about 60-120 nm.
5. The composition of embodiment 1, 2, 3, or 4, wherein the aggregates have a fractal dimension of about 1.9-2.1.
6. The composition of embodiment 1, 2, 3, 4, or 5, wherein the aggregates comprise particles of the insoluble alpha-glucan with an average size of about 5-25 nm.
7. The composition of embodiment 1, 2, 3, 4, 5, or 6, wherein the individual molecules of the insoluble alpha-glucan each have a weight-average degree of polymerization (DPw) of at least about 600.
8. The composition of embodiment 1, 2, 3, 4, 5, 6, or 7, comprising agglomerates of the aggregates, wherein the agglomerates have an average size of about 1-200 microns.
9. The composition of embodiment 8, wherein the agglomerates have an average size of about 1-110 microns.
10. The composition of embodiment 1, 2, 3, 4, 5, 6, 7, 8, or 9, wherein the insoluble alpha-glucan has at least 50% alpha-1,3-glycosidic linkages.
11. The composition of embodiment 10, wherein the insoluble alpha-glucan has at least 90% alpha-1,3-glycosidic linkages.
12. The composition of embodiment 1, 2, 3, 4, 5, 6, 7, 8, 9, 10, or 11, wherein the composition is in the form of a household product, personal care product, pharmaceutical product, industrial product, or food product.
13. A method of producing aggregates of insoluble alpha-glucan according to any of embodiments 1-11, the method comprising: (a) contacting at least water, sucrose, and a glucosyltransferase enzyme that synthesizes insoluble alpha-glucan at a yield of at least about 40%; and (b) preparing a dispersion of the insoluble alpha-glucan produced in step (a).
14. The method of embodiment 13, wherein step (b) comprises preparing a wet cake of the insoluble alpha-glucan produced in step (a), and dispersing the wet cake in water or an aqueous solution.
15. A composition comprising aggregates of insoluble alpha-glucan produced according to the method of embodiment 13 or 14.

### EXAMPLES

The present disclosure is further exemplified in the following Examples. It should be understood that these Examples, while indicating certain preferred aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Example 1

### Non-Native Glucosyltransferases that Produce Insoluble Alpha-1,3-Glucan at High Yield

This Example describes identifying non-native glucosyltransferase enzymes that synthesize insoluble alpha-glucan at a yield of at least 40%.

The amino acid sequence of the glucosyltransferase used to prepare amino acid substitutions in this Example was SEQ ID NO:4 (GTF 6855), which essentially is an N-terminally truncated (signal peptide and variable region removed) version of the full-length wild type glucosyltransferase (represented by SEQ ID NO:62) from Streptococcus salivarius SK126 (see Table 1). Substitutions made in SEQ ID NO:4 can be characterized as substituting for native amino acid residues, as each amino acid residue/position of SEQ ID NO:4 (apart from the Met-1 residue of SEQ ID NO:4) corresponds accordingly with an amino acid residue/position within SEQ ID NO:62. In reactions comprising at least sucrose and water, the glucosyltransferase of SEQ ID NO:4 typically produces alpha-glucan having about 100% alpha-1,3 linkages and a DP_{w} of 400 or greater (e.g., refer to U.S. Pat. Nos. 8,871,474 and 9,169,506, and U.S. Pat. Appl. Publ. No. 2017/0002336, which are incorporated herein by reference). This alpha-glucan product, which is insoluble, can be isolated following enzymatic synthesis via filtration, for example.

### Single Amino Acid Substitutions

GTF 6855 variants (each with a single amino acid substitution as indicated in Table 3) from site evaluation libraries (SEL) were each bacterially expressed, purified, and normalized to a concentration of 100 ppm. Each enzyme preparation was then screened (in triplicate) using sucrose as substrate in alpha-1,3-glucan synthesis reactions. In addition to determining the amount of alpha-1,3-glucan polymer produced in each reaction, the soluble sugar products (fructose, glucose, leucrose, gluco-oligosaccharides) and residual sucrose of each reaction were analyzed by HPLC after about a 20-hour incubation.

Each GTF (GTF 6855 and each variant thereof) was entered into a reaction with sucrose to determine yield and selectivity. Each reaction was performed as follows: 37.5 μL of 100 ppm enzyme sample (ppm based on a BSA calibration curve) was added to 262.5 μL of 86 g/L sucrose (75 g/L final) in 20 mM Na₂HPO₄/NaH₂PO₄ pH 5.7 and incubated overnight (about 20 hours) at 30° C. After this incubation, each reaction was quenched by incubation for 1 hour at 80° C. A 200-4 aliquot of each quenched reaction was filtered in vacuo via a 0.45-μm filter plate (Millipore 0.45-μm Hydrophilic) and each filtrate was diluted 5× (10 μL sample+40 μL 20 mM Na₂HPO₄/NaH₂PO₄) in preparation for HPLC sugar analysis. The profiles of these reactions (˜20 hours) are provided in Table 3.

TABLE 3
Product Profiles of GTF 6855 (SEQ ID NO: 4) and
Single Amino Acid-Substituted Variants thereof

| | | | | | | | |
|---|---|---|---|---|---|---|---|
| | | | | | | Alpha-1,3- | |
| | Sucrose | Leucrose | Glucose | Fructose | Oligomers | Glucan^{f} | Fructose |
| GTF | (g/L)^{d} | (g/L)^{d} | (g/L)^{d} | (g/L)^{d} | (g/L)^{d,e} | Yieldⁱ | Balance |

Plate 1^{a}

| | | | | | | | |
|---|---|---|---|---|---|---|---|
| 6855^{b} | 1.6 | 21.1 | 6.3 | 28.9 | 9.1 | 31% | 97% |
| 6855^{b} | 1.6 | 21.3 | 6.3 | 29.1 | 10.5 | 27% | 98% |
| 6855^{b} | 1.6 | 21.2 | 6.3 | 29.3 | 10.0 | 29% | 98% |
| 6855^{b} | 1.6 | 21.1 | 6.3 | 28.9 | 10.8 | 27% | 97% |
| V186A^{c} | 1.6 | 21.3 | 6.4 | 28.8 | 10.7 | 27% | 97% |
| V186M | 1.6 | 21.4 | 6.4 | 28.7 | 10.6 | 27% | 97% |
| E194C | 1.6 | 21.2 | 6.3 | 29.0 | 9.4 | 30% | 98% |
| L434N | 1.9 | 22.7 | 7.1 | 28.4 | 12.7 | 18% | 99% |
| A472C | 31.0 | 2.6 | 2.5 | 23.8 | 4.6 | 38% | 99% |
| A472S | 5.3 | 2.8 | 13.9 | 36.5 | 9.1 | 31% | 97% |
| A510E | 8.5 | 5.4 | 5.5 | 34.5 | 5.6 | 53% | 100% |
| A510E | 1.9 | 6.5 | 5.6 | 36.7 | 6.1 | 58% | 98% |
| A510I | 4.3 | 6.8 | 5.4 | 35.2 | 5.4 | 57% | 98% |
| A510V | 1.7 | 9.5 | 6.4 | 35.6 | 6.8 | 51% | 99% |
| L513Y | 1.4 | 10.3 | 4.2 | 35.3 | 7.2 | 54% | 99% |
| M529L | 1.9 | 10.4 | 4.2 | 35.2 | 10.9 | 44% | 99% |
| K578M | 1.6 | 21.0 | 6.4 | 28.8 | 10.8 | 27% | 97% |
| Y605W | 6.1 | 8.0 | 2.6 | 33.3 | 5.4 | 59% | 97% |
| F607N | 8.4 | 11.4 | 4.1 | 30.5 | 7.1 | 45% | 98% |
| F607W | 9.1 | 4.6 | 3.8 | 33.9 | 8.6 | 49% | 98% |
| N613I | 4.5 | 7.7 | 6.4 | 35.8 | 14.8 | 29% | 101% |
| N613M | 2.7 | 11.0 | 5.3 | 34.6 | 12.1 | 37% | 100% |
| N613T | 1.7 | 10.3 | 4.6 | 35.0 | 7.1 | 53% | 98% |
| N613V | 2.8 | 0.0 | 6.3 | 37.3 | 12.1 | 48% | 92% |
| Q616E | 3.9 | 2.4 | 5.8 | 37.3 | 8.8 | 53% | 97% |
| K625A | 1.5 | 21.2 | 6.3 | 29.4 | 9.9 | 29% | 99% |
| K625M | 1.5 | 21.3 | 6.3 | 29.3 | 10.6 | 27% | 99% |
| S631T | 5.4 | 11.4 | 4.6 | 32.0 | 7.6 | 46% | 97% |
| T635H | 4.1 | 11.0 | 5.0 | 32.7 | 8.2 | 46% | 97% |
| T635W | 13.1 | 8.5 | 4.5 | 29.6 | 7.0 | 42% | 98% |
| I636H | 7.0 | 11.7 | 5.0 | 31.1 | 8.1 | 42% | 98% |
| D947G | 2.4 | 19.1 | 6.1 | 29.8 | 9.9 | 31% | 98% |
| F951Y | 4.0 | 1.5 | 9.9 | 38.0 | 15.4 | 28% | 97% |
| E849M | 1.4 | 20.7 | 6.2 | 29.5 | 10.4 | 29% | 98% |
| Q1007A | 1.4 | 19.4 | 6.2 | 30.2 | 10.1 | 31% | 98% |
| D1003G | 13.8 | 10.7 | 4.6 | 28.3 | 5.4 | 42% | 98% |
| A1022M | 1.7 | 20.6 | 6.2 | 29.3 | 12.2 | 24% | 98% |
| D1028L | 1.6 | 22.1 | 6.6 | 28.9 | 11.6 | 23% | 99% |
| D1028Q | 1.6 | 21.7 | 6.5 | 29.4 | 10.9 | 26% | 99% |
| A1057H | 1.5 | 21.4 | 6.4 | 29.2 | 10.6 | 27% | 98% |
| N1096A | 1.6 | 22.4 | 6.6 | 28.6 | 10.7 | 25% | 98% |
| E1132A | 1.5 | 21.4 | 6.4 | 29.2 | 10.6 | 27% | 98% |
| E1132H | 1.5 | 21.3 | 6.4 | 29.2 | 10.5 | 27% | 98% |
| E1132K | 1.5 | 21.4 | 6.4 | 29.2 | 10.4 | 27% | 98% |
| E1132R | 1.5 | 21.6 | 6.4 | 29.1 | 10.8 | 26% | 99% |
| L1212N | 1.5 | 20.9 | 6.3 | 29.5 | 10.4 | 28% | 98% |
| T1431M | 1.5 | 21.4 | 6.3 | 29.4 | 10.5 | 27% | 99% |
| A1442R | 1.5 | 21.3 | 6.4 | 29.1 | 10.6 | 27% | 98% |
| Dead^{g} | 79.4 | 0.0 | 0.0 | 0.0 | 0.0 | 0% | 100% |
| Blank^{h} | 79.7 | 0.0 | 0.1 | 0.0 | 0.0 | 0% | 100% |
| Blank^{h} | 80.1 | 0.0 | 0.0 | 0.0 | 0.0 | 0% | 100% |

Plate 2^{a}

| | | | | | | | |
|---|---|---|---|---|---|---|---|
| 6855^{b} | 1.4 | 20.1 | 6.4 | 28.2 | 10.0 | 29% | 99% |
| 6855^{b} | 1.4 | 20.1 | 6.4 | 28.2 | 10.1 | 28% | 99% |
| 6855^{b} | 1.4 | 20.0 | 6.3 | 28.3 | 10.3 | 28% | 99% |
| 6855^{b} | 1.5 | 20.2 | 6.3 | 28.2 | 10.0 | 29% | 100% |
| Y219C^{c} | 1.5 | 20.6 | 6.5 | 27.7 | 10.7 | 25% | 99% |
| E243H | 1.4 | 20.3 | 6.3 | 28.2 | 10.1 | 28% | 100% |
| L373A | 2.4 | 11.3 | 11.2 | 27.4 | 21.6 | −7% | 87% |
| L373Q | 4.0 | 7.5 | 10.7 | 28.4 | 21.5 | −2% | 87% |
| L373V | 2.5 | 11.6 | 11.5 | 27.5 | 21.8 | −9% | 88% |
| A377I | 2.9 | 15.5 | 6.6 | 29.3 | 11.3 | 29% | 98% |
| D425Q | 1.8 | 15.3 | 5.3 | 30.3 | 9.6 | 39% | 99% |
| L428V | 5.3 | 10.5 | 6.2 | 30.8 | 8.2 | 42% | 98% |
| N475F | 6.1 | 26.8 | 20.5 | 24.9 | 7.2 | −16% | 106% |
| N475W | 1.5 | 61.8 | 7.5 | 9.1 | 1.9 | −8% | 106% |
| L513F | 1.0 | 10.9 | 4.6 | 33.3 | 7.1 | 55% | 99% |
| L513W | 1.3 | 11.5 | 4.9 | 32.4 | 8.9 | 48% | 98% |
| M529N | 3.5 | 11.6 | 4.8 | 31.6 | 7.6 | 49% | 99% |
| I608Y | 2.4 | 15.7 | 5.7 | 29.9 | 9.8 | 35% | 99% |
| N613G | 2.2 | 10.5 | 5.0 | 33.5 | 10.6 | 43% | 101% |
| N613L | 2.9 | 13.3 | 5.0 | 32.1 | 11.7 | 35% | 102% |
| D617E | 8.4 | 10.2 | 6.9 | 29.8 | 9.0 | 34% | 99% |
| E621T | 1.5 | 18.6 | 6.0 | 29.1 | 10.4 | 30% | 100% |
| I623H | 69.8 | 0.2 | 1.4 | 3.3 | 0.0 | 4% | 101% |
| I627W | 7.7 | 12.2 | 5.2 | 28.9 | 7.9 | 40% | 99% |
| S631D | 9.8 | 12.3 | 5.7 | 27.5 | 8.0 | 35% | 98% |
| S631E | 10.1 | 12.6 | 5.6 | 27.3 | 8.0 | 35% | 99% |
| S631R | 6.7 | 12.3 | 5.4 | 28.7 | 8.1 | 40% | 97% |
| G633W | 7.0 | 7.2 | 5.5 | 31.9 | 8.5 | 46% | 99% |
| F634A | 7.4 | 8.4 | 5.7 | 30.8 | 8.2 | 43% | 98% |
| T635E | 1.6 | 17.2 | 6.0 | 29.9 | 9.5 | 35% | 100% |
| T635I | 1.5 | 17.4 | 6.2 | 30.5 | 10.1 | 32% | 102% |
| T635Y | 13.8 | 8.0 | 4.6 | 28.0 | 6.7 | 43% | 99% |
| A510E | 2.5 | 5.9 | 5.5 | 34.8 | 4.3 | 66% | 99% |
| N904E | 5.7 | 6.9 | 12.6 | 32.5 | 13.5 | 15% | 98% |
| K930G | 1.4 | 19.8 | 6.2 | 28.4 | 10.0 | 30% | 99% |
| K930V | 1.4 | 19.6 | 6.3 | 28.6 | 10.0 | 30% | 100% |
| D947F | 1.4 | 20.3 | 6.2 | 27.8 | 9.9 | 29% | 99% |
| D947I | 1.4 | 19.9 | 6.3 | 28.6 | 10.7 | 27% | 100% |
| D947K | 1.4 | 19.9 | 6.2 | 28.6 | 9.7 | 30% | 100% |
| D947N | 1.4 | 20.5 | 6.3 | 27.9 | 10.0 | 28% | 99% |
| D947Q | 1.4 | 19.5 | 6.2 | 28.4 | 9.6 | 31% | 99% |
| D947S | 1.3 | 18.9 | 6.1 | 28.8 | 9.4 | 33% | 99% |
| D947V | 1.4 | 19.8 | 6.2 | 28.3 | 9.7 | 30% | 99% |
| D947Y | 1.4 | 20.7 | 6.3 | 28.1 | 10.0 | 28% | 100% |
| Q1007S | 1.3 | 18.3 | 6.1 | 29.1 | 9.6 | 33% | 99% |
| D1003N | 3.6 | 13.1 | 5.7 | 30.5 | 9.8 | 38% | 99% |
| I1026H | 1.4 | 19.4 | 6.2 | 28.7 | 9.7 | 31% | 100% |
| D1028A | 1.5 | 20.1 | 6.5 | 28.4 | 10.8 | 26% | 100% |
| D1028M | 1.5 | 20.4 | 6.6 | 28.1 | 11.1 | 24% | 100% |
| V1037A | 1.5 | 20.2 | 6.4 | 28.4 | 10.3 | 28% | 100% |
| K1041A | 4.3 | 19.6 | 6.5 | 27.0 | 10.7 | 23% | 99% |
| K1041M | 1.5 | 20.5 | 6.4 | 28.0 | 10.5 | 26% | 100% |
| D1080M | 1.4 | 20.0 | 6.4 | 28.3 | 10.1 | 29% | 99% |
| F1244P | 1.4 | 19.6 | 6.3 | 28.6 | 9.9 | 30% | 100% |
| F1244Q | 1.4 | 19.7 | 6.4 | 28.6 | 9.9 | 30% | 100% |
| T1431Q | 1.4 | 20.0 | 6.2 | 28.5 | 8.9 | 33% | 100% |
| G1484P | 1.5 | 20.1 | 6.3 | 28.5 | 9.2 | 31% | 100% |
| W1437N | 1.4 | 19.5 | 6.0 | 28.9 | 8.4 | 35% | 100% |
| Dead^{g} | 75.5 | 0.0 | 0.0 | 0.0 | 0.0 | 0% | 100% |
| Blank^{h} | 75.5 | 0.0 | 0.0 | 0.0 | 0.0 | 0% | 100% |
| Blank^{h} | 76.0 | 0.0 | 0.0 | 0.0 | 0.5 | −2% | 100% |

^{a}Glucan synthesis reactions were run in microtiter plate format (two plates).
^{b}GTF 6855, SEQ ID NO: 4. Reactions with this GTF were run in quadruplicate per plate.
^{c}Each listed GTF with a substitution is a version of GTF 6855 comprising a substitution at a respective position, where the position number is in correspondence with the residue numbering of SEQ ID NO: 62. The wild type residue is listed first (before residue position number) and the substituting residue is listed second (after the residue position number) (this “wild type residue-position number-variant residue” annotation format applies throughout the present disclosure).
^{d}Sucrose, leucrose, glucose, fructose and oligomers were measured as present in filtrate prepared post reaction.
^{e}“Oligomers”, gluco-oligosaccharides (believed to all or mostly be of DP ≤ 7 or 8).
^{f}Insoluble alpha-1,3-glucan product.
^{g}GTF with destroyed activity was entered into the reaction.
^{h}No GTF was added to the reaction.
ⁱAlpha-glucan yield based on glucosyl.

Table 3 indicates examples of amino acid substitutions that can be employed for producing a non-native glucosyltransferase useful herein for synthesizing insoluble alpha-glucan at a yield of at least 40%.

### Multiple Amino Acid Substitutions

Briefly, certain combinations of amino acid substitutions were made to SEQ ID NO:4 (GTF 6855). These substitutions are listed in Tables 4 and 5 below.

Each variant enzyme of Table 4 was entered into a glucan synthesis reaction with parameters that were the same as, or similar to, the following: vessel, 250-mL indented shake flask agitated at 120 rpm; initial pH, 5.7; reaction volume, 50 mL; sucrose, 75 g/L; GTF, 1.5 mL lysate of E. coli cells heterologously expressing enzyme; KH₂PO4, 20 mM; temperature, 30° C.; time, about 20-24 hours.

Each variant enzyme of Table 5 was entered into a glucan synthesis reaction with parameters that were the same as, or similar to, the following: vessel, 500-mL jacketed reactor with Teflon®-pitched blade turbine (45-degree angle) on a glass stir rod and agitated at 50-200 rpm; initial pH, 5.5; reaction volume, 500 mL; sucrose, 108 g/L; KH₂PO₄, 1 mM; temperature, 39° C.; time, about 18-24 hours; filtrate from a previous alpha-1,3-glucan synthesis reaction, 50 vol %.

The alpha-1,3-glucan yields of these reactions (measured similarly as above) are provided in Tables 4 and 5.

TABLE 4
Alpha-1,3-Glucan Yields of GTF 6855 (SEQ ID N0: 4)
Variants with Multiple Amino Acid Substitutions

| | Alpha-1,3- |
|---|---|
| GTF^{a} | Glucan^{b} Yield^{c} |
| A510D/F607Y/R741S | 72.6% |
| A510D/F607Y/N743S | 79.2% |
| A510D/F607Y/D948G | 88.2% |
| A510D/R741S/D948G | 74.5% |
| A510D/F607Y/R741S/D948G | 82.8% |
| A510E/F607Y/R741S/R1172C | 78.2% |
| A510D/F607Y/D820G/D948G | 87.8% |
| A510D/F607Y/D948G/R1172C | 88.6% |
| A510D/F607Y/N743S/D948G/R1172C | 89.4% |
| A510D/F607Y/R741S/L784Q/F929L/R1172C | 79.3% |

^{a}Each listed GTF is a version of GTF 6855 (SEQ ID NO: 4) comprising substitutions at respective positions, where each position number is in correspondence with the residue numbering of SEQ ID NO: 62.
^{b}Insoluble alpha-1,3-glucan product.
^{c}Alpha-1,3-glucan yield based on glucosyl.

TABLE 5
Alpha-1,3-Glucan Yields of GTF 6855 (SEQ ID NO: 4) Variants with Multiple Amino Acid Substitutions

| | | | | | | | | | | | | | Alpha-1,3-Glucan^{b} |
|---|---|---|---|---|---|---|---|---|---|---|---|---|---|
| GTF^{a} | | | | | | | | | | | | | Yield^{c} |
| A510D | Q588L | F607Y | R741S | D948G | | | R722H | T877K | | M1253I | K1277N | | 88% |
| A510D | Q588L | F607Y | R741S | D948G | | | R722H | T877K | V1188E | M1253I | Q957P | | 92% |
| A510D | Q588L | F607Y | R741S | D948G | | | | T877K | V1188E | M1253I | Q957P | | 91% |
| A510D | Q588L | F607Y | R741S | D948G | | | | | | M1253I | | | 89% |
| A510D | Q588L | F607W | R741S | D948G | | | | | | | | | 91% |
| | Q588L | F607Y | R741S | D948G | | | | | | | | | 91% |
| A510D | Q588L | F607Y | R741S | D948G | N628D | T635A | | T877K | | M1253I | F929L | R1172C | 92% |
| A510D | Q588L | F607W | R741S | D948G | S631T | S710G | R722H | T877K | V1188E | M1253I | | | 94% |
| A510D | Q588L | F607W | R741S | D948G | S631T | S710G | R722H | T877K | V1188E | | | | 93% |
| A510D | Q588L | F607W | R741S | D948G | S631T | S710G | | T877K | V1188E | M1253I | | | 96% |
| A510D | Q588L | F607Y | R741S | D948G | | | | | | | | | 89% |
| A510D | Q588L | F607Y | R741S | D948G | | | | | V1188E | | | | 88% |
| A510D | Q588L | F607W | R741S | D948G | S631T | S710G | | | V1188E | | | | 96% |
| A510D | Q588L | F607W | R741S | D948G | | S710G | R722H | T877K | | M1253I | | | 96% |
| A510D | Q588L | F607Y | R741S | D948G | S631T | | R722H | T877K | V1188E | M1253I | | | 96% |
| A510D | Q588L | F607W | R741S | D948G | S631T | | | T877K | V1188E | M1253I | | | 94% |
| A510D | Q588L | F607W | R741S | D948G | S631T | | | | V1188E | | | | 98% |
| A510D | Q588L | F607Y | R741S | D948G | S631T | | R722H | T877K | V1188E | M1253I | | | 95% |
| A510D | Q588L | F607W | R741S | D948G | | | | | V1188E | M1253I | | | 93% |

^{a}Each listed GTF is a version of GTF 6855 (SEQ ID NO: 4) comprising substitutions at respective positions, where each position number is in correspondence with the residue numbering of SEQ ID NO: 62.
^{b}Insoluble alpha-1,3-glucan product.
^{c}Alpha-1,3-glucan yield based on glucosyl.

Tables 4 and 5 indicate examples of sets of amino acid substitutions that can be employed for producing a non-native glucosyltransferase useful herein for synthesizing insoluble alpha-glucan at a yield of at least 40%.

Thus, non-native glucosyltransferase enzymes that synthesize insoluble alpha-glucan at a yield of at least 40% were identified. These enzymes can be used, for example, to synthesize insoluble alpha-glucan for producing aggregates herein, such as shown in Example 2 below.

### Example 2

### Preparation and Analysis of Aggregates and Agglomerates of Insoluble Alpha-Glucan

This Example describes preparing and analyzing insoluble alpha-glucan aggregates. These aggregates were prepared by dispersing insoluble alpha-1,3-glucan that was synthesized in reactions catalyzed by a non-native glucosyltransferases as described in Example 1 (above).

Glucan synthesis reactions were prepared using glucosyltransferase enzymes as described in Example 1 that produce insoluble alpha-1,3-glucan at a yield between 50-95% (based on the glucosyl component of the reaction). The reactions were performed in a manner similar to what is described in Example 1. Following each reaction, samples of each alpha-1,3-glucan product (insoluble, about 100% alpha-1,3 linkages) were filtered, washed to remove most fructose and other residuals sugars, and processed to about 10-40 wt % solids using a filter apparatus. The resulting glucan filter cakes (“wet cakes) contained about 90-75 wt % water, and were not dried. In some cases, filter cakes were found to have about 0.4 wt % sugars.

Dispersions of individual alpha-1,3-glucan products were prepared by vortexing wet cake that had been diluted with water (100:1 water:sample). Each dispersion was then processed and analyzed as follows.

The dispersion (20 μL) was deposited on freshly cleaved mica, allowed to sit for 30 seconds, and then spun off at 5000 rpm for 30 seconds. Atomic force microscopy (AFM) was then used to image the aggregated structure that was deposited. Though this imaging initially seemed to show spherical particles that are highly aggregated, it was appreciated upon further analysis that the aggregated particles include extended rod-like particles. The primary particles could be measured by a line scan across the image, using a series of bumps for the height profile. From this profile, it was possible to measure the lateral particle size to be about 30 nm. Since this measurement was convoluted by the lateral width of the probe, it was very likely an overestimate. The AFM image also showed that the particles are uniform in size.

A second sample was prepared similarly on a transmission electron microscopy (TEM) grid, depositing particles by drying a dispersion. The TEM image showed aggregates of spherical and rod-like particles. The size of these particles appeared rather uniform. The spherical particles were estimated to be about 6.5 nm in diameter from the image. An unequivocal measurement was difficult because the particles were highly aggregated from being excessively dried in the vacuum of the instrument. Undoubtedly, because of the preparation conditions, the original high surface area and very open structure of the as-polymerized glucan had collapsed to form a highly aggregated structure, which was then recorded in the image. This TEM result was useful to show that some of the primary particles are spherical, uniform in size, and about 10 nm in diameter. However, it is cautioned that an exact measurement of size was difficult due to structural collapse, which obscures the limits between particles and very likely changes the size of individual particles.

TEM images for aggregated alpha-1,3-glucan were compared to TEM images for carbon black and fumed silica. The silica particles showed severe aggregation, appeared larger (˜15 nm) than the particles of glucan, and showed larger polydispersity. The comparison between alpha-1,3-glucan and carbon black demonstrated their similarity in particle size, but the carbon black samples were about 30 nm in diameter with higher polydispersity. The strongest conclusion that could be drawn from the comparison of TEM images of alpha-1,3-glucan, carbon black and silica is that the particle shape of these three materials is spherical. These observations notwithstanding, it has since been appreciated (above) that alpha-1,3-glucan particles of aggregates can also be rod-like in shape.

The TEM images for the spherical particles of alpha-1,3-glucan, carbon black and silica were compared to the platelet particles formed by enzymatically polymerized cellulose, which were produced as disclosed in U.S. Patent Appl. Publ. No. 2017/0327857 and Int. Patent Appl. Publ. No. WO2016106011 (both references incorporated herein by reference). The degree of polymerization of the cellulose chains was low (˜15 DPw). A similar procedure (as above) of sample preparation was carried out by drying a dispersion of the cellulose. The enzymatically polymerized cellulose formed platelets upon drying on the flat mica surface. The platelets laid flat on the surface and appeared faceted. The flat surfaces of the platelets were large and asymmetric, of ˜100 nm in size and with an aspect ratio larger than 2:1. These flat particles were unmistakably non-spherical. While films of the enzymatically polymerized cellulose material were transparent, films formed by alpha-1,3-glucan were opaque. This difference is due to the aggregates and agglomerates of alpha-1,3-glucan, which scatter light and give rise to the observed opacity. However, the transparency of the cellulose films is due to the perfection in the stacking of platelets that occurred during film formation. There is little doubt from the information that has been provided above that the primary particle shape of alpha-1,3-glucan is spherical. These observations notwithstanding, it has since been appreciated (above) that alpha-1,3-glucan particles of aggregates can also be rod-like in shape.

Small angle X-ray and neutron scattering (SAXS, SANS) are analytical techniques that can be used to measure the size of primary particles and aggregates. Unlike microscopy, scattering averages over a large volume (˜1 mm³) as compared to the much smaller volumes sampled in high resolution microscopy measurements (˜10⁻⁹ mm³). Although microscopy is useful to identify particle shape, scattering is useful to measure average particle size. The use of a regularization and maximum entropy methods to measure particle size from SAXS data is well documented (Ilaysky and Jemian, 2009, J. Appl. Cryst. 42:347-353; Jemian et al., 1991, Acta Metall. Mater. 39:2477-2487; both incorporated herein by reference). SAXS data were collected from 2-wt % dispersions of alpha-1,3-glucan prepared following the above methodology. Double logarithmic plots were prepared of scattered intensity vs the scattering vector magnitude, 0.0015≤q(=4π sin θ/λ)≤0.4 Å⁻¹, for incident X-ray radiation of wavelength λ scattered by an angle 2θ. The samples included in this study are listed in Table 6. A similar scattering profile was observed for all of these samples. The data at the largest q corresponds to scattering from the smallest features in the material. A well-defined Porod range with terminal dependence of l˜q⁴ was observed within the range 0.05≤q≤0.2 Å⁻¹. This dependence is due to the presence of particles with sharp interfaces. A well-defined Guinier region (knee) was observed within the range 0.02≤q≤0.05 Å¹, which is due to a primary particle size of about 15 nm in diameter, as specified in more detail below. Within the range 0.002≤q≤0.02 Å¹, a dependence of l˜q² was observed, corresponding to the scattering from aggregates within the length scale of 10 to 100 nm. The maximum entropy method (Ilaysky and Jemian, 2009; Jemian et al., 1991) was applied to these data. The data within the range 0.01≤q·0.1 Å⁻¹ were included in this analysis, bracketing the Guinier range specified earlier. The volume particle size distribution functions obtained from this procedure approximated a log-normal shape, which had a Gaussian profile in a linear-log plot of volume distribution versus particle diameter. The median values for each sample are tabulated in Table 6. The average of all the tabulated median values is 12.3 nm. This measurement of primary particle size is intermediate between the AFM value of 30 nm and the TEM value of 6.5 nm (above). The full-width-at-half-height of these distributions was ˜14 nm yielding an estimate for the dispersity of ±7 nm. The dispersity of ±7 nm is an overestimate caused by the Fourier transform procedure. Values of size and dispersity are generated below using an alternative method.

TABLE 6
Particle Size of Aqqreqated Alpha-1,3-Glucan

| | Alpha-1 3-Glucan^{a} | | |
|---|---|---|---|
| | | | Median Particle |
| Production Scale & Notes | Yield^{b} | DPw | Diameter (nm) |
| Pilot plant | 75-90% | 761 | 14 |
| Pilot plant | 75-90% | 683 | 13.4 |
| Lab | 75-90% | 827 | 12.9 |
| 4th recycle of oligomers^{c} | | | |
| Lab | 75-90% | | 12.9 |
| Lab | 75-90% | 873 | 12.5 |
| Batch reaction | | | |
| Lab | 75-90% | 821 | 12.5 |
| Batch reaction, recycled oligomers_{c} | | | |
| Lab | 75-90% | | 12.5 |
| | | | 11.5 |
| Pilot plant | 90-95% | 1168 | 11.5 |
| First batch, no oligomer recycle | | | |
| Pilot plant | 50-60% | 760 | 10.2 |
| 25 wt. % wet cake | | | |

^{a}Insoluble alpha-1,3-glucan product.
^{b}Range of alpha-1,3-glucan yield (based on glucosyl) estimated in view of the non-native glucosyltransferase used. A yield of 75-90% was obtained using a non-native glucosyltransferase from Table 4. A yield of 90-95% was obtained using a non-native glucosyltransferase from Table 5.
^{c}Oligosaccharide (oligomer) byproducts from an earlier alpha-1,3-glucan synthesis reaction were entered into the respective reaction using filtrate isolated from the earlier reaction.

Data were collected within a broad range of scattering vector magnitude, 3×10⁻⁵≤q≤0.4 Å⁻¹, corresponding to a range in length scale from 1 nm to 10 μm. Small angle and ultra-small angle instruments operating with X-ray or neutron sources were used to collect these data (SAXS, USAXS, SANS, USANS). These instruments were located at the synchrotron at the Advanced Photon Source near Chicago, Ill., at the NIST Center for Neutron Scattering in Gaithersburg, Md., and at the Experimental Station in Wilmington, Del. The data at the largest q corresponds to scattering from the smallest features in the material. A well-defined Porod range with terminal dependence of 1˜q⁻⁴ was observed within the range 0.05≤q≤0.2 Å⁻¹. This dependence is due to the presence of particles with sharp interfaces. A well-defined Guinier region (knee) was observed within the range 0.02≤q≤0.05 Å⁻¹, which is due to the primary particle size of about 15 nm in diameter. Within the range 0.002≤q≤0.02 Å⁻¹, a dependence of l˜q⁻² was observed, corresponding to the scattering from aggregates within the length scale of 10 to 100 nm. Within the range 3×10⁻⁵≤q≤0.002 Å⁻¹, a dependence of l˜q⁻³ was observed, corresponding to the scattering from agglomerates within the size range of 100 nm to 10 μm. The internal structure of the aggregates and agglomerates can be deduced from the slopes of the double log plots, which are identified as the fractal dimensions of these scattering objects. While the aggregates within the length scale of 10 to 100 nm are arborescent in nature, the agglomerates are space filling.

Data from the samples listed on Table 7 were fitted according to the universal fit method of Beaucage (1995, J. Appl. Cryst. 28:717-728; incorporated herein by reference). The radius of gyration of the primary particle (Rg1) was one of the parameters that was fitted. The fractal dimension, FD2, was also determined by this procedure and the results for this parameter are discussed below. These parameters are tabulated in Table 7.

TABLE 7
Fractal Dimension (FD2) of Aggregated Alpha-1,3-Glucan

| Production Scale | Alpha-1,3-Glucan^{a} | | | |
|---|---|---|---|---|
| & Notes | Yield^{b} | DPw | Rg1 (Å) | FD2^{c} |
| Pilot plant | 90-95% | 1168 | 59.0 +/− 0.6 | 2.435 +/− 0.022 |
| First batch, no | | | | |
| oligomer recycle | | | | |
| Pilot plant | 90-95% | 1168 | 51.96 +/− 0.62 | 3.08 +/− 0.11 |
| First batch, no | | | | |
| oligomer recycle | | | | |
| Pilot plant | 90-95% | 959 | 54.33 +/− 0.82 | 3.17 +/− 0.13 |
| Pilot plant | 90-95% | 892 | 66.9 +/− 2.9 | 2.273 +/− 0.046 |
| 10-16 wt. % wet | | | | |
| cake | | | | |
| Pilot plant | 90-95% | 761 | 65.0 +/− 3.4 | 2.386 +/− 0.076 |
| 40 wt. % wet cake | | | | |
| Pilot plant | 90-95% | 761 | 62.0 +/− 1.3 | 2.465 +/− 0.081 |
| 40 wt. % wet cake | | | | |
| Pilot plant | 90-95% | 727 | 65.0 +/− 3.6 | 2.211 +/− 0.046 |
| 10-16 wt. % wet | | | | |
| cake | | | | |
| Pilot plant | 90-95% | 783 | 65.3 +/− 2.8 | 2.456 +/− 0.065 |
| 40 wt. % wet cake | | | | |
| Pilot plant | 90-95% | 793 | 66.7 +/− 3.1 | 2.287 +/− 0.068 |
| 10-16 wt. % wet | | | | |
| cake | | | | |
| Pilot plant | 90-95% | 793 | 61.1 +/− 1.3 | 2.267 +/− 0.025 |
| 10-16 wt. % wet | | | | |
| cake | | | | |
| Pilot plant | 90-95% | 793 | 65.3 +/− 2.1 | 2.509 +/− 0.089 |
| 40 wt. % wet cake | | | | |
| Pilot plant ~10.1 | 90-95% | 793 | 56.34 +/− 0.81 | 3.10 +/− 0.16 |
| wt. % wet cake | | | | |
| Pilot plant | 90-95% | 784 | 64.4 +/− 4.5 | 2.238 +/− 0.041 |
| 10-16 wt. % wet | | | | |
| cake | | | | |
| Pilot plant | 90-95% | 1351 | 61.7 +/− 3.5 | 2.03 +/− 0.12 |
| 10-16 wt. % wet | | | | |
| cake | | | | |
| Pilot plant | 90-95% | 1351 | 54 +/− 3 | 2.26 +/− 0.12 |
| 40 wt. % wet cake | | | | |
| Pilot plant | 90-95% | 1119 | 67.5 +/− 4.6 | 2.615 +/− 0.079 |
| Pilot plant | 90-95% | 1130 | 49.45 +/− 0.31 | 2.581 +/− 0.033 |
| Pilot plant | 90-95% | 1130 | 49.73 +/− 0.24 | 2.92 +/− 0.04 |
| Pilot plant | 90-95% | 683 | 58.76 +/− 0.33 | 3.247 +/− 0.065 |
| Pilot plant | 80-85% | 1223 | 50.7 +/− 1.1 | 2.918 +/− 0.071 |
| Pilot plant | 80-85% | 778 | 59.13 +/− 0.31 | 2.450 +/− 0.038 |
| Pilot plant | 80-85% | 1298 | 47.90 +/− 0.18 | 3.283 +/− 0.045 |
| Pilot plant | 80-85% | 858 | 58.2 +/− 0.4 | 3.345 +/− 0.059 |
| Pilot plant | 80-85% | 862 | 58.07 +/− 0.31 | 3.131 +/− 0.054 |
| Pilot plant | 80-85% | 761 | 63.15 +/− 0.42 | 2.633 +/− 0.045 |
| Pilot plant | 80-85% | 1319 | 55.67 +/− 0.55 | 2.918 +/− 0.054 |
| Pilot plant | 50-60% | 760 | 55.81 +/− 0.57 | 2.936 +/− 0.054 |
| 25 wt. % wet cake | | | | |
| Lab | 80-85% | | 73.1 +/− 3.6 | 2.242 +/− 0.048 |

^{a}Insoluble alpha-1,3-glucan product.
^{b}Range of alpha-1,3-glucan yield (based on glucosyl) estimated in view of the non-native glucosyltransferase used.
^{c}Fractal dimension of aggregates within the length scale of 10 to 100 nm.

The data fit was performed several times and the errors quoted in Table 7 for the fitted parameters were determined from the root mean square deviation of the fitted values. A box plot for Rg1 shows that the two central quartiles lie between 5.5 and 6.5 nm, and therefore shows that Rg1 is narrowly distributed around 6 nm. Assuming the particle shape is spherical, the diameter of the primary particle size, D, is 15.5±1.5 nm; the quoted error corresponds to ±the standard deviation of the tabulated values. For a spherical particle, the relationship between radius of gyration and diameter is:

$$D = 2 \sqrt{\frac{5}{3}} {R}_{g} .$$

If a single alpha-1,3-glucan chain is assumed to have a degree of polymerization of 800 DPw, then the number of chains per primary particle is ˜10±3, as shown in Table 8. The number of 800 DPw chains per primary particle may be as large as ˜13 for a particle size of 15.5 nm or as low as 6 chains for a particle size of 12.3 nm, which was the diameter determined above from the maximum entropy method. For an intermediate diameter of 14.3 nm, the calculated number of 800 DPw chains per globule is 10. In the calculation of the number of chains per primary particle, it was assumed that the particle was a spherical globule with density of 1.4 g/cc, which is an estimate (˜85%) for the density of amorphous alpha-1,3-glucan based on a crystalline density of 1.6 g/cc. These calculations notwithstanding, it has since been appreciated (above) that alpha-1,3-glucan particles of aggregates can also be rod-like in shape.

TABLE 8
Number of Polymer Chains in Alpha-1,3-Glucan Primary
Particles with Different Diameters (D)

| D (nm) | No. of 800 DPw chains |
|---|---|
| 15.5 | 12.7 |
| 12.3 | 6.3 |
| 14.3 | 10.0 |

Values of FD2, the fractal dimension of aggregates within the length scale of 10 to 100 nm, were tabulated in Table 7. A frequency plot of the parameter FD2 was constructed. The aggregate fractal dimension is roughly uniformly distributed above 2, but no values below 2 were recorded. Typical arborescent structures from computer simulations of aggregates, as formed by diffusion of primary spherical particles, are shown on FIG. 1. The structures differ slightly depending on whether the aggregation process is limited by diffusion or by the aggregation process. Their typical fractal dimensions are respectively 1.71 and 2.3, but variations within these values occur depending on the assumptions of the simulation. The arborescent structures and fractal dimensions from these simulation results are typical of what is observed in colloid aggregation, such as with fumed silica or carbon black. Therefore, by analogous reasoning based on the results of colloid aggregation, it is possible to deduct that no values below 2 were observed (in Table 7) because the limiting value of ˜2 is imposed by the structure of the glucan aggregate. The size distribution of aggregates is very likely highly polydisperse. The fitted values of fractal dimension that were higher than 2 could have arisen from agglomerates of small aggregates within the length scale of 10 to 100 nm, biasing the fits towards larger values of FD2.

High shear (microfluidizer shear rate ˜10⁷ s⁻¹) was applied to a dispersed alpha-1,3-glucan sample featuring a large intensity within the agglomerate region. Before shear, a dependence of l˜q⁻² was observed within the ranges 5×10⁻³≤q≤0.02 Å⁻¹, and a dependence of l˜q⁻⁴ was observed within the range 3×10⁻⁴≤q≤5×10⁻³ Å. After shear, a dependence of l˜q⁻² was observed within the full range of 3×10⁻⁴≤q≤0.02 Å⁻¹, and the l˜q⁻⁴ dependence disappeared. Shear had the effect of lowering the agglomerate intensity while the range of scattering arising from aggregates, with a dependence of l˜q⁻², was expanded to lower q for at least a decade. These results showed that the agglomerates are comparatively labile relative to the aggregates. Shear had the effect of dispersing the agglomerates, while the aggregates persisted after the shear was applied.

Light scattering results from a Mastersizer® 2000 (Malvern) also support the hierarchical model of the alpha-1,3-glucan structure, as shown on Table 9. The agglomerate particle size of an unsonicated glucan sample (˜20 μm) was reduced by about an order of magnitude (to ˜3 μm) on sonication for 3 minutes (9 kJ/kg) in 15 mL of deionized water.

TABLE 9
Light Scattering Analysis of Alpha-1,3-
Glucan^{a} before and after Sonication

| | Agglomerate | |
|---|---|---|
| Sonication | Size | Instrument |
| None | 20 μm | Laser |
| | | Diffraction |
| 3 minute horn | 3 μm | Laser |
| | | Diffraction |

^{a}Same glucan as sample in Table 7 with Rg1 of 58.2 +/− 0.4.

Sonication in general had the effect of decreasing floc and agglomerate size as measured by light scattering. An alpha-1,3-glucan dispersion prepared and sonicated as above (3 minutes at 9 kJ/kg) resulted in agglomerates of about 4.3 μm median diameter (D50); before sonication, the D50 was 21.1 μm. Agglomerate size as a function of sonication power showed that a minimum specific energy of about 10 kJ/kg could be necessary to break up very large alpha-1,3-glucan agglomerates, which were recorded to have a D50 of about 25 μm before sonication. As the specific energy of sonication was increased, starting at about 10 kJ/kg, the measured agglomerate D50 decreased and stabilized at about 5 μm at power settings greater than 1 MJ/kg. A higher frequency of sonication had a larger effect on measured agglomerate size. Overall, these results show that alpha-1,3-glucan agglomerates are highly polydisperse and labile, and their size can be affected by shear and sonication.

The internal structure of alpha-1,3-glucan dispersions was analyzed by various detectors that were either coupled to a liquid chromatography system (on-line) or were used without a column (off-line), as specified in Table 10.

TABLE 10
Specifications of Detection Systems

| Technique | Quantity | On-line | Off-line |
|---|---|---|---|
| Multiangle (static) Light | M, R_{g} | ✓ | ✓ |
| Scattering (MALS) | | | |
| Dynamic Light Scattering | Rₕ, PSD | ✓ | ✓ |
| (DLS) | | | |
| Evaporative Light Scattering | cₙ | ✓ | |
| Detector (ELSD) | | | |

M—Molar Mass
R_{g}—Radius of gyration
Rₕ—Hydrodynamic Radius
PSD—Particle Size Distribution of glucan dispersions
cₙ—Particle Mass Concentration
c_{c}—Polymer Mass Concentration
On-line - Technique used with injection into a column
Off-line - Technique used without a column

The chromatography system was equipped with a silica-based monolithic column for size separation up to 1 micron in hydrodynamic chromatography mode. Such a monolithic column comprises a bimodal porous structure that achieves, in one column, both high permeability (2-3 μm diameter) and high surface area (diameter up to 50 nm).

Dispersions of 0.1 mg/ml alpha-1,3-glucan were sonicated (9 kJ/kg) for 40 minutes. Aggregate size distributions (PSD's) were measured off-line by DLS at 0, 360 and 1440 minutes after sonication. The measured PSD's were narrow (dispersity of ˜±15%) and respectively distributed about mean particle size values (in Rₕ) of 111, 180, and 271 nm. Filtration of such a dispersion through a 1.2-μm Acrodick™ membrane onto a tarred aluminum plate demonstrated the loss of only 10% material.

The polysaccharide dispersion with Rₕ=180 nm (per DLS) was injected into the triple detection chromatographic system equipped with monolith column and three on-line detectors listed in Table 10. For comparison, two dispersions of polystyrene spheres that differed in particle diameter (300 nm and 400 nm) were also prepared at a concentration of 0.1 mg/m and injected into the same chromatographic system. For a solid sphere with diameter D,

$${R}_{g} = \frac{D}{2} \sqrt{\frac{3}{5}} , and {R}_{h} = \frac{{R}_{g}}{0.8} .$$

Therefore, spheres of 400 nm and 300 nm in diameter have Rₕ=194 nm and 145 nm, respectively. It was observed that the smaller polystyrene particles (300 nm) eluted later from the column, followed by glucan dispersion and larger particles (400 nm), confirming the separation by size in hydrodynamic chromatography mode.

The internal structure (fractal dimension) of the sonicated (40-minutes) alpha-1,3-glucan dispersion was characterized by the structural parameter

$$ρ = \frac{{R}_{g}}{{R}_{h}} ,$$

which varies inversely to fractal dimension. A shown in Table 11, ρ increases for more open structures, which have a lower fractal dimension. Additionally, the angular dependence of static light scattered intensity can also be used to characterize the conformation of dispersed objects.

TABLE 11
Structural Parameter ρ

| Conformation | ρ = R_{g}/Rₕ |
|---|---|
| Rod, Axial Ratio 1:100 | 2.8 |
| Rod, Axial Ratio 1:25 | 2.1 |
| Random Coil, Good Solvent | 1.8 |
| Random Coil, Theta Solvent | 1.5 |
| Randomly Branched | 1.7 |
| Hyperbranched | 1.2 |
| Homogeneous Sphere | 0.8 |
| Core-Shell Sphere | 0.5-0.8 |
| Amylose | 1.6-2.2 |
| Amylopectin | 1.0-1.3 |
| Pullulan | 1.6-1.8 |
| Polyvinylacetate Microgel | 0.6 |

As an example of conformational analysis via the structural parameter ρ and the angular dependence of scattered intensity, two very different scattering objects were compared. A dispersion of 60-nm diameter polystyrene (PS) spheres was prepared at 0.1 mg/mL. This dispersion of hard spheres was compared to a dispersion of 400-kDa pullulan chains at 0.1 mg/m L. These dispersions, comprising objects of very different fractal dimension, were separated by the monolithic column technique (above). Plots of scattered intensity divided by concentration versus

$${sin}^{2} ( \frac{θ}{2} )$$

were prepared from the MALS data collected at the apex of the elution profile, for light scattered by an angle θ. In the solid sphere case, the apex occurred at 18.6 minutes of elution time, and for the pullulan chains the apex was at 19.4 minutes. The scattering profiles were accordingly fitted (ASTRA™ software, Wyatt Technologies, Santa Barbara, Calif.) very well by models of a solid sphere, for the PS sphere case, and a random coil, for the pullulan case, as expected. The coefficient of determination was higher than R²=0.99 for both fits. Measurements of R_{g} (Rₕ) from MALS (DLS) for the polystyrene spheres were 26.4 (34) nm, yielding ρ=0.78, which agrees with the expected value of 0.8 from Table 9. For pullulan R_{g} (Rₕ) values of 24 (14) nm, yield a value of ρ=1.7, describing an open structure and in agreement with the value corresponding to a random coil from Table 11.

Monolithic separation of sonicated alpha-1,3-glucan dispersions was tested by the same method as the dispersions of PS spheres and pullulan (above). An alpha-1,3-glucan dispersion prepared at 0.1 mg/mL was analyzed six hours after it was sonicated (9 kJ/kg) for 40 minutes. Measurements yielded mean values of R_{g} (Rₕ) from MALS (DLS) of 356 (201.3) nm and ρ=1.77. The PSD was very narrow, and the scattering profile fitted very well to the functional form for a random coil (R²=0.99) and did not fit at all with the functional form for a solid sphere. These findings show that the structure alpha-1,3-glucan aggregates herein is very open with a low fractal dimension.

What is claimed is:

1. A composition comprising aggregates of insoluble alpha-glucan, wherein the aggregates have an average hydrodynamic radius of about 50-300 nm and a fractal dimension of about 1.6-2.4, and the insoluble alpha-glucan comprises alpha-1,3-glycosidic linkages.

2. The composition of claim 1, wherein the aggregates are arborescent.

3. The composition of claim 1, wherein the aggregates have an average hydrodynamic radius of about 50-150 nm.

4. The composition of claim 1, wherein the aggregates have an average hydrodynamic radius of about 60-120 nm.

5. The composition of claim 1, wherein the aggregates have a fractal dimension of about 1.9-2.1.

6. The composition of claim 1, wherein the aggregates comprise particles of the insoluble alpha-glucan with an average size of about 5-25 nm.

7. The composition of claim 6, wherein the individual molecules of the insoluble alpha-glucan each have a weight-average degree of polymerization (DPw) of at least about 600.

8. The composition of claim 1, comprising agglomerates of said aggregates, wherein the agglomerates have an average size of about 1-200 microns.

9. The composition of claim 8, wherein the agglomerates have an average size of about 1-110 microns.

10. The composition of claim 1, wherein the insoluble alpha-glucan has at least 50% alpha-1,3-glycosidic linkages.

11. The composition of claim 10, wherein the insoluble alpha-glucan has at least 90% alpha-1,3-glycosidic linkages.

12. The composition of claim 1, wherein the composition is in the form of a household product, personal care product, pharmaceutical product, industrial product, or food product.

13. A method of producing aggregates of insoluble alpha-glucan according to claim 1, said method comprising:
(a) contacting at least water, sucrose, and a glucosyltransferase enzyme that synthesizes insoluble alpha-glucan at a yield of at least about 40%; and
(b) preparing a dispersion of the insoluble alpha-glucan produced in step (a).

14. The method of claim 13, wherein step (b) comprises preparing a wet cake of the insoluble alpha-glucan produced in step (a), and dispersing the wet cake in water or an aqueous solution.

15. A composition comprising aggregates of insoluble alpha-glucan produced according to the method of claim 13.


SEQUENCE LISTING (65 sequences)

SEQ ID NO: 1 - DNA, 4308 bp, Streptococcus sobrinus
atggttgacggcaaatactactattatgatcaggacggtaacgtaaagaagaatttcgcggtgagcgttggtgacaaaatctactacttcgatgaaactggtgcatataaggataccagcaaagtggacgccgacaagagcagcagcgcggttagccaaaacgcgaccatctttgcggcgaataaccgtgcgtacagcacctctgcaaagaattttgaagcggtggataactacctgaccgcagacagctggtatcgtccgaaatccatcctgaaggacggcaaaacctggaccgagagcggtaaggatgatttccgtccactgctgatggcatggtggcctgacaccgaaactaagcgcaactacgtgaactatatgaataaagtggtcggtattgacaagacgtacactgcggaaacgtcgcaagcggatttgaccgcagcggcggagctggttcaagcgcgtatcgagcagaagattaccagcgaaaacaacaccaaatggctgcgcgaagcaatctccgcgttcgttaagacgcagcctcagtggaacggcgagtccgaaaagccgtatgacgatcacttgcagaacggtgcgctgctgtttgataaccaaaccgacctgacgccagacacccaaagcaattaccgtttgctgaaccgtaccccgaccaatcagactggtagcctggatagccgttttacgtataatccgaatgacccgttgggcggctacgatttcttgctggcgaacgacgttgacaatagcaatccggtcgtccaggctgaacagttgaactggctgcattatctgctgaactttggctctatttacgctaacgatgccgacgccaattttgacagcattcgcgttgatgccgtcgataatgtcgatgctgatctgctgcaaatcagcagcgattacctgaaagcagcgtatggcatcgacaagaataacaagaatgcgaacaaccatgttagcatcgtcgaagcgtggagcgacaatgataccccgtatttgcacgacgatggcgataatctgatgaacatggacaacaaatttcgcctgtccatgctgtggagcctggcaaagccgctggacaaacgtagcggtttgaacccgctgattcacaatagcctggtggaccgcgaggtggacgatcgtgaagtggaaaccgtgccgtcctacagctttgctcgtgcacatgatagcgaggtgcaggacatcatccgtgacattatcaaggctgagattaacccaaatagctttggttatagcttcactcaagaagagatcgagcaagcctttaagatttacaacgaggatttgaagaaaacggacaagaaatacacccactacaatgtgccgctgagctacaccctgctgctgaccaacaagggcagcatcccgcgtgtgtactatggtgatatgttcaccgatgatggccaatacatggcaaacaagaccgtcaactacgacgcaatcgagagcctgctgaaagcccgtatgaaatatgtcagcggtggccaagcaatgcagaactatcaaattggtaatggcgagattttgaccagcgtgcgctatggtaaaggtgccctgaagcagagcgataagggtgacgcgacgacgcgcactagcggtgttggcgtggttatgggtaatcagccgaacttctccctggacggtaaagttgtggccctgaatatgggtgcggcccatgcgaatcaagaataccgtgcactgatggtcagcactaaagacggtgtggcaacttacgcaaccgatgctgacgcatccaaagcgggcctggtcaagcgtaccgacgagaacggctacctgtacttcctgaatgatgatctgaagggcgtcgcgaaccctcaggtttccggcttcttgcaagtgtgggttccagttggtgccgccgatgaccaggacattcgcgtcgccgccagcgacacggcgagcacggatggtaaaagcctgcatcaagatgcggcgatggacagccgcgtcatgtttgagggtttcagcaattttcaatccttcgcgaccaaagaagaagaatacacgaatgttgttatcgcgaacaatgtcgataagttcgttagctggggtatcaccgattttgaaatggctccgcagtatgttagcagcaccgacggtcagttcttggacagcgtcatccagaatggctatgcgtttactgatcgctatgatctgggtatgtccaaggcgaacaagtatggcacggcagaccaactggttaaggcaatcaaagccctgcacgctaaaggcctgaaagttatggcggactgggtcccggatcaaatgtacacctttccaaaacaggaagttgtgaccgttacccgcaccgacaaattcggtaaaccgatcgccggctctcaaatcaatcacagcttgtatgtgaccgacaccaaatccagcggcgacgactaccaagcgaagtacggcggtgccttcctggatgaactgaaagaaaagtacccggaactgttcacgaaaaagcaaattagcacgggccaagcgattgatccgagcgtgaaaatcaagcagtggagcgcaaaatacttcaatggttcgaatatcctgggtcgcggtgcggactatgtgctgagcgaccaggtcagcaataagtatttcaacgtggcgagcgacaccttgttcctgccgtccagcctgctgggcaaggtcgtggagagcggcattcgttacgacggcaagggttacatctacaacagctccgcgaccggcgatcaggtcaaagcgtctttcattacggaagccggtaacctgtattacttcggcaaagacggttacatggttactggtgcccagacgattaatggcgccaactacttcttcctggaaaacggtacggcactgcgtaatacgatttacaccgatgctcaaggtaatagccactattacgcgaatgatggcaaacgctatgaaaatggctatcaacagttcggtaacgattggcgctactttaaagatggtaacatggcagtcggcctgaccacggttgatggcaacgtgcaatactttgacaaagacggcgtccaggcaaaggataagattatcgtcacccgtgatggcaaggtccgttacttcgatcagcacaacggtaacgcggcgaccaacacgttcattgctgataaaactggccattggtattacctgggtaaagatggcgtcgcggtgactggcgcccagaccgtcggcaaacaaaaactgtacttcgaggccaacggtcaacaagttaaaggtgactttgttacgtccgatgagggcaaactgtatttctatgacgttgattctggtgacatgtggacggacaccttcatcgaggataaggcgggcaactggttctatttgggcaaggatggtgcggcagttacgggtgcccaaacgattcgcggtcagaagctgtacttcaaggccaatggtcaacaggtcaagggtgacattgttaagggcaccgacggtaaaatccgctactatgatgcaaaatccggtgaacaggtgttcaacaaaacggtgaaagctgcggatggcaaaacgtatgttatcggtaatgatggtgtcgcggtggaccctagcgtggttaaaggtcaaacctttaaggacgcttcgggcgctctgcgtttctacaacttgaagggtcaactggtcactggcagcggctggtatgaaaccgcgaaccatgactgggtttacattcagtccggcaaggcactgaccggcgaacagaccattaacggtcaacacctgtatttcaaagaagatggtcaccaagtcaagggtcagttggtcacgggcaccgatggtaaagtgcgttactatgacgccaacagcggtgaccaagcattcaacaagagcgtcactgtgaatggtaaaacctattactttggcaacgatggtacggcgcagactgctggcaacccgaagggtcagacgttcaaggatggctccgacatccgtttttactctatggaaggccaactggtgaccggctcgggttggtacgagaacgcgcaaggccagtggctgtatgtgaaaaacggtaaggtgctgactggtctgcaaaccgttggcagccagcgtgtttacttcgacgagaatggtattcaggccaagggcaaagcagtgcgtaccagcgatggcaaaattcgttatttcgacgaaaacagcggcagcatgatcacgaatcaatggaagttcgtctatggtcagtattactactttggtaacgacggtgcacgtatttaccgtggttggaactaa

SEQ ID NO: 2 - protein, 1435 aa, Streptococcus sobrinus
MVDGKYYYYDQDGNVKKNFAVSVGDKIYYFDETGAYKDTSKVDADKSSSAVSQNATIFAANNRAYSTSAKNFEAVDNYLTADSWYRPKSILKDGKTWTESGKDDFRPLLMAWWPDTETKRNYVNYMNKVVGIDKTYTAETSQADLTAAAELVQARIEQKITSENNTKWLREAISAFVKTQPQWNGESEKPYDDHLQNGALLFDNQTDLTPDTQSNYRLLNRTPTNQTGSLDSRFTYNPNDPLGGYDFLLANDVDNSNPVVQAEQLNWLHYLLNFGSIYANDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIEQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAATNTFIADKTGHWYYLGKDGVAVTGAQTVGKQKLYFEANGQQVKGDFVTSDEGKLYFYDVDSGDMWTDTFIEDKAGNWFYLGKDGAAVTGAQTIRGQKLYFKANGQQVKGDIVKGTDGKIRYYDAKSGEQVFNKTVKAADGKTYVIGNDGVAVDPSVVKGQTFKDASGALRFYNLKGQLVTGSGWYETANHDWVYIQSGKALTGEQTINGQHLYFKEDGHQVKGQLVTGTDGKVRYYDANSGDQAFNKSVTVNGKTYYFGNDGTAQTAGNPKGQTFKDGSDIRFYSMEGQLVTGSGWYENAQGQWLYVKNGKVLTGLQTVGSQRVYFDENGIQAKGKAVRTSDGKIRYFDENSGSMITNQWKFVYGQYYYFGNDGARIYRGWN

SEQ ID NO: 3 - DNA, 4026 bp, Streptococcus salivarius SK126
atgatcgacggcaaatactattatgttaatgaggacggtagccacaaagaaaactttgcgattacggttaatggtcaactgctgtatttcggtaaggacggcgcactgacctctagcagcacttacagctttaccccaggtacgacgaacatcgtggatggcttttctatcaacaaccgcgcgtatgactccagcgaagcgtcctttgaactgattgatggctacttgactgccgactcctggtatcgtccggcttccatcatcaaggacggtgtcacgtggcaggccagcaccgcagaggactttcgcccgctgctgatggcgtggtggccaaacgtggatacccaggtgaactatctgaactacatgtctaaagtgtttaacctggacgcaaagtatagcagcaccgataaacaagagactctgaaggttgcagctaaggatattcagattaagatcgagcagaaaattcaggcggagaaaagcacccaatggctgcgcgaaacgatcagcgcttttgtgaaaacccaaccacagtggaacaaagagactgagaattactcgaaaggtggtggtgaggatcatctgcaaggcggtgcactgctgtacgtgaatgatagccgtaccccgtgggcaaatagcgattatcgccgcctgaaccgcaccgctaccaatcaaacgggtacgattgacaagtccattctggacgagcagagcgacccaaatcacatgggcggtttcgacttcctgctggcgaatgatgttgacctgtccaacccggttgtgcaggcagagcagctgaaccagattcactacttgatgaattggggctctatcgtgatgggtgacaaagacgcaaactttgatggtatccgtgtcgatgcagttgacaacgtcgatgccgacatgctgcaactgtataccaactacttccgtgaatactacggtgttaacaaaagcgaagcgaacgcactggcgcacattagcgttttggaagcgtggagcttgaatgataatcactacaacgacaaaaccgatggtgcagcattggcgatggagaataagcagcgtctggcgctgctgtttagcctggctaaaccgattaaagagcgcaccccggcagtgagcccgctgtataacaacaccttcaatacgacccaacgcgatgagaaaaccgactggatcaataaagacggttctaaggcctataacgaggatggtactgtgaagcagagcaccattggtaagtacaatgaaaaatatggtgatgcatcgggcaattatgtgttcatccgtgcccatgataacaatgtccaagacatcattgcggagatcattaagaaagaaatcaacccgaaaagcgatggtttcaccatcactgacgccgaaatgaaacaagcgttcgagatttacaataaggacatgctgagcagcgacaagaagtacaccctgaataacatcccggcagcttatgccgtgatgttgcagaacatggaaacgattacccgtgtctattatggtgacctgtacaccgacgacggccactacatggaaaccaagtccccgtattacgacaccatcgttaacctgatgaaaagccgtatcaagtacgtcagcggtggccaggcccaacgtagctactggctgccgaccgacggcaagatggacaatagcgacgttgagctgtatcgcaccaacgaagtgtataccagcgtccgttacggtaaagacattatgaccgcgaacgataccgagggtagcaagtacagccgcaccagcggccaggtcaccctggttgcaaacaacccgaagctgaccctggaccagagcgcgaagctgaatgtggaaatgggtaagattcacgcgaatcagaaataccgtgccctgattgtgggcacggctgacggtatcaagaatttcaccagcgacgcagatgctatcgcggcaggctacgtgaaagaaaccgactccaatggcgttctgacttttggcgctaatgacatcaaaggttatgaaaccttcgacatgtccggctttgttgctgtttgggtgccggtcggcgcgagcgatgatcaggacattcgtgtcgctcctagcactgaggccaagaaagagggtgaattgaccctgaaagcgaccgaagcatacgattcccagctgatctatgaaggttttagcaattttcaaaccatcccggatggtagcgacccgagcgtgtacaccaatcgcaagatcgcagagaacgtggacctgttcaagtcctggggtgttacctcgtttgaaatggcaccgcagttcgtttccgcagatgatggcacttttctggactctgtgatccaaaacggctatgcgtttgccgatcgttacgatttggcgatgagcaagaacaacaaatacggcagcaaagaggacttgcgtgacgcgctgaaagccctgcataaagcaggcatccaggcgattgcagactgggtcccggaccagatttatcagttgccgggcaaagaagtggtcacggcgactcgcaccgacggcgcaggccgtaaaatcgcggacgcgatcattgatcatagcctgtacgttgcgaacactaagagcagcggcaaagattaccaggcgaagtacggtggtgagttcttggcggagctgaaggccaagtacccggagatgttcaaagtgaacatgatttctaccggcaaaccgattgatgacagcgtcaaactgaaacagtggaaagcagaatactttaacggcaccaacgtcttggagcgcggtgtgggttatgtcctgagcgatgaagccacgggtaaatactttaccgtcacgaaggatggcaacttcattccgttgcagctgacgggtaatgagaaagtcgtgaccggctttagcaatgatggcaaaggtatcacctacttcggtacgagcggcactcaagcgaaatctgcgttcgttacgttcaatggtaatacttactattttgacgctcgtggtcacatggttacgaacggcgagtattcgccgaacggtaaggatgtttaccgtttcctgccgaatggtattatgctgtctaacgctttttacgttgatgcaaatggtaacacgtacctgtacaacagcaagggccaaatgtacaaaggcggttacaccaaatttgacgttaccgaaacggacaaagatggtaaggaaagcaaggtggtgaagtttcgttactttacgaacgaaggtgtcatggcaaaaggcgttaccgtgattgacggcttcacgcaatactttggtgaagatggtttccaagcgaaagacaagctggtcacgttcaagggcaagacgtactacttcgatgcacacaccggcaatgcgatcaaggacacctggcgtaatatcaatggcaagtggtatcatttcgacgcgaacggcgttgcagcgaccggcgctcaggtcatcaatggccaaaaactgtatttcaacgaggacggcagccaagtgaaaggcggtgttgtcaaaaacgcggacggtacgtattctaaatacaaagagggttctggtgaactggttaccaacgagttcttcacgacggatggcaatgtttggtactacgcaggcgcgaatggcaagaccgttacgggtgcccaggtgattaacggccaacacctgtacttcaatgcggacggttcgcaagtgaagggcggtgtggtcaagaacgcggatggcacctatagcaaatatgatgcgtctaccggcgaacgcctgaccaatgagtttttcaccacgggtgataacaactggtactacattggcgcaaacggcaagagcgtgacgggcgaggtcaagatcggtgacgatacctatttctttgccaaagatggcaagcaagttaagggtcaaactgtcagcgcgggtaacggtcgtattagctactactatggtgatagcggtaagcgtgcggtgagcacttggatcgaaatccaaccgggtgtttatgtctacttcgacaagaacggcattgcctatccgcctcgtgtgctgaattaa

SEQ ID NO: 4 - protein, 1341 aa, Streptococcus salivarius SK126 [sequence omitted]

SEQ ID NO: 5 - DNA, 3744 bp, Streptococcus salivarius
atgccaagccacattaagaccatcaacggcaaacaatactacgtggaggatgacggtacgattcgcaagaattacgtcctggagcgtatcggtggcagccaatactttaatgcagaaaccggtgaactgtctaatcagaaagagtatcgtttcgacaaaaatggtggtactggtagcagcgcggacagcacgaacaccaacgtgactgtgaacggtgacaaaaacgcattttacggtaccacggacaaagacattgagctggtcgacggctatttcaccgcgaacacctggtatcgcccgaaagaaatcctgaaagacggcaaagaatggaccgccagcacggagaacgataaacgcccgctgctgaccgtctggtggcctagcaaagcaatccaggcgtcttatctgaactacatgaaagagcaaggcctgggtaccaaccaaacgtacacgagcttctccagccaaacccaaatggatcaagcagccctggaagtgcaaaagcgtattgaagagcgcatcgcacgcgagggcaataccgactggctgcgcacgaccatcaagaacttcgtgaaaacccaaccgggttggaacagcacctctgaaaatctggacaataatgatcatctgcaaggtggcgccctgctgtacaataacgactcccgcacgagccacgcgaacagcgactatcgcctgctgaatcgtacgccgaccagccagaccggcaaacacaatccgaaatacaccaaagataccagcaatggtggtttcgaatttctgctggcgaacgacatcgataactctaatccggcggttcaagcagagcaactgaactggctgcattacattatgaacatcggtaccatcacgggcggttctgaggatgaaaacttcgacggcgttcgtgttgacgctgtggataatgtgaatgcggatctgctgcaaatcgcgagcgactatttcaaagcaaaatacggtgctgatcaaagccaagatcaggcgatcaaacacttgagcatcctggaagcgtggtcccataacgacgcctactataacgaagataccaaaggcgcgcagttgccgatggatgatccgatgcacctggctctggtctactcgctgctgcgtccgatcggcaatcgcagcggtgtggaaccgctgatttccaacagcctgaatgaccgtagcgagtccggtaagaacagcaaacgtatggcgaactacgcgttcgtacgcgcgcatgatagcgaggtgcaatcgattattggccagatcatcaaaaacgagatcaatccgcaaagcaccggtaatacgttcaccctggatgagatgaagaaagcgtttgagatttacaacaaggatatgcgtagcgcgaataagcagtatacgcagtacaacatcccgagcgcgtatgcgttgatgctgacccacaaggataccgttccgcgtgtgtattacggtgatatgtatacggacgacggtcagtacatggcgcaaaagagcccatactatgatgcgatcgaaacgctgctgaaaggtcgcatccgctatgccgcaggtggtcaggacatgaaggtcaactatattggttacggtaacactaacggctgggatgctgcgggcgtgctgaccagcgtacgttatggcacgggcgcaaatagcgccagcgatacgggtaccgccgaaacgcgtaatcaaggtatggcagtgattgttagcaaccaaccggcgctgcgtctgactagcaatttgaccattaacatgggtgccgcacaccgtaatcaggcttaccgtccgctgctgctgacgaccaacgatggcgtcgcgacctatttgaacgatagcgatgcgaatggtatcgttaagtacaccgacggtaatggtaatctgaccttctccgcaaacgagattcgtggcatccgtaacccgcaagttgatggctatctggccgtctgggttccggtaggtgcgtcggagaatcaggatgttcgtgtggcgccgagcaaagagaagaacagctccggtctggtttacgagagcaatgctgccctggatagccaagttatctacgaaggcttcagcaacttccaggacttcgttcagaatccgagccagtataccaacaaaaagattgcagagaatgcaaatttgttcaaatcctggggtattaccagctttgaatttgcgccgcagtacgtgagctcggatgatggtagcttcctggacagcgttattcagaacggttatgcgtttacggaccgctacgacattggtatgagcaaagacaacaaatatggttcgctggcggatttgaaggcagcactgaagagcttgcatgccgttggtattagcgcaatcgcggattgggttcctgatcagatctacaatctgccaggcgacgaggtcgtcaccgcaacccgcgttaacaactacggcgaaaccaaagatggtgcaatcattgatcactctttgtacgcggccaaaacccgtacttttggtaacgactaccagggtaagtatggtggtgcgttcctggacgagctgaaacgtctgtatccgcagatctttgaccgcgttcagatttctaccggtaagcgcatgaccacggacgagaagatcacccaatggtctgcaaagtatatgaacggtacgaacatcttggaccgtggctctgaatacgttttgaagaatggtctgaatggttactatggcaccaatggtggcaaagtttcgctgccgaaagttgtgggtagcaatcaaagcacgaatggcgacaatcaaaacggcgacggtagcggcaagtttgaaaagcgtctgttcagcgtgcgttaccgttataacaatggccagtacgcgaaaaatgcctttatcaaagataacgacggcaatgtttactatttcgacaatagcggtcgtatggctgtcggtgagaaaacgattgacggcaagcagtacttcttcctggctaatggcgttcagctgcgtgacggctaccgtcaaaatcgtcgcggtcaggtgttttactacgaccagaatggtgtgctgaacgcaaacggtaaacaagacccgaagcctgacaacaataacaatgcgagcggccgtaatcaattcgtccagatcggtaacaacgtgtgggcgtattatgatggcaatggtaaacgtgtcaccggtcaccagaacatcaacggtcaggagttgtttttcgataacaacggtgtccaggttaagggtcgtacggtgaatgagaacggtgcaattcgctactatgacgcgaatagcggtgagatggcacgcaatcgtttcgcggagattgaaccgggcgtctgggcatactttaacaatgacggcaccgcagtgaagggttctcagaatatcaatggtcaagacctgtacttcgaccagaacggtcgtcaggtcaagggtgcgctggccaatgttgatggcaacctgcgctattacgacgttaacagcggtgagctgtaccgtaatcgtttccacgaaatcgacggcagctggtattactttgatggtaacggtaatgcggtgaagggtatggtcaatatcaacggccaaaatctgttgtttgacaataacggcaaacagattaagggtcatctggtccgcgtcaacggcgtcgtgcgctattttgatccgaactctggtgaaatggcggttaatcgttgggttgaggtgagcccaggttggtgggtttactttgacggtgaaggtcgtggtcagatctaa

SEQ ID NO: 6 - protein, 1247 aa, Streptococcus salivarius
MPSHIKTINGKQYYVEDDGTIRKNYVLERIGGSQYFNAETGELSNQKEYRFDKNGGTGSSADSTNTNVTVNGDKNAFYGTTDKDIELVDGYFTANTWYRPKEILKDGKEWTASTENDKRPLLTVWWPSKAIQASYLNYMKEQGLGTNQTYTSFSSQTQMDQAALEVQKRIEERIAREGNTDWLRTTIKNFVKTQPGWNSTSENLDNNDHLQGGALLYNNDSRTSHANSDYRLLNRTPTSQTGKHNPKYTKDTSNGGFEFLLANDIDNSNPAVQAEQLNWLHYIMNIGTITGGSEDENFDGVRVDAVDNVNADLLQIASDYFKAKYGADQSQDQAIKHLSILEAWSHNDAYYNEDTKGAQLPMDDPMHLALVYSLLRPIGNRSGVEPLISNSLNDRSESGKNSKRMANYAFVRAHDSEVQSIIGQIIKNEINPQSTGNTFTLDEMKKAFEIYNKDMRSANKQYTQYNIPSAYALMLTHKDTVPRVYYGDMYTDDGQYMAQKSPYYDAIETLLKGRIRYAAGGQDMKVNYIGYGNTNGWDAAGVLTSVRYGTGANSASDTGTAETRNQGMAVIVSNQPALRLTSNLTINMGAAHRNQAYRPLLLTTNDGVATYLNDSDANGIVKYTDGNGNLTFSANEIRGIRNPQVDGYLAVWVPVGASENQDVRVAPSKEKNSSGLVYESNAALDSQVIYEGFSNFQDFVQNPSQYTNKKIAENANLFKSWGITSFEFAPQYVSSDDGSFLDSVIQNGYAFTDRYDIGMSKDNKYGSLADLKAALKSLHAVGISAIADWVPDQIYNLPGDEVVTATRVNNYGETKDGAIIDHSLYAAKTRTFGNDYQGKYGGAFLDELKRLYPQIFDRVQISTGKRMTTDEKITQWSAKYMNGTNILDRGSEYVLKNGLNGYYGTNGGKVSLPKVVGSNQSTNGDNQNGDGSGKFEKRLFSVRYRYNNGQYAKNAFIKDNDGNVYYFDNSGRMAVGEKTIDGKQYFFLANGVQLRDGYRQNRRGQVFYYDQNGVLNANGKQDPKPDNNNNASGRNQFVQIGNNVWAYYDGNGKRVTGHQNINGQELFFDNNGVQVKGRTVNENGAIRYYDANSGEMARNRFAEIEPGVWAYFNNDGTAVKGSQNINGQDLYFDQNGRQVKGALANVDGNLRYYDVNSGELYRNRFHEIDGSWYYFDGNGNAVKGMVNINGQNLLFDNNGKQIKGHLVRVNGVVRYFDPNSGEMAVNRWVEVSPGWWVYFDGEGRGQI

SEQ ID NO: 7 - DNA, 4434 bp, Streptococcus salivarius
atggacgaaacgcaggataagaccgtgacgcagagcaacagcggcaccaccgcttccctggtcactagccctgaagccacgaaagaggcggacaaacgcacgaacactaaagaggccgacgttctgacgcctgcaaaagaaacgaacgcagtcgagactgcgaccaccactaacacccaggcgacggcggaggccgccacgaccgcgaccaccgcggacgtcgcggtggctgcggtgccgaacaaagaagcggtcgttaccacggatgctccggcggtcacgaccgagaaagcggaagaacagccggctaccgttaaagcagaagtcgtcaatacggaagtgaaagcgccggaagcggctctgaaagacagcgaggttgaggcagcgctgagcctgaagaacatcaagaacattgatggcaagtattactatgttaatgaggatggcagccacaaagagaatttcgctattaccgtgaatggccagctgctgtactttggtaaagacggtgcgctgacgtcctctagcacgtattcttttaccccaggcactaccaatatcgtggacggttttagcattaacaaccgcgcttacgacagcagcgaggcgagctttgagctgatcgacggttacttgaccgcagacagctggtatcgtccggctagcatcatcaaagatggtgttacgtggcaagcgtccaccgccgaggattttcgtccgctgctgatggcatggtggccgaatgtggatacgcaggtgaactatttgaattacatgtccaaagttttcaacctggacgcgaaatactctagcaccgacaaacaggaaaccctgaaagtggcagcaaaagacattcaaatcaagattgaacaaaagattcaagcggagaagagcacgcagtggctgcgtgaaactatcagcgcctttgtgaaaacccagccgcagtggaacaaagaaaccgagaattacagcaagggtggtggtgaggaccacctgcaaggtggcgcactgctgtatgttaacgacagccgtaccccttgggcgaatagcgattaccgtcgtctgaatcgcaccgcaaccaatcagacgggcacgatcgataagtctattctggacgagcagtctgacccaaaccacatgggcggtttcgactttctgctggcgaacgacgtcgacctgagcaatccggtcgtgcaggctgagcagctgaatcaaatccactatctgatgaattggggttccattgtgatgggtgacaaggatgcgaactttgacggcattcgtgtcgatgcagttgacaacgtggacgcggacatgttgcaactgtataccaattacttccgtgagtactacggtgtgaacaagagcgaagctaacgcactggctcacatcagcgttctggaggcgtggagcctgaatgataatcattacaatgacaagaccgatggtgcggcactggcaatggagaataagcaacgtctggcgctgttgttttcgttggcgaaaccgatcaaagagcgtaccccggcagtgagcccgctgtataacaacaccttcaataccacccagcgtgatgaaaagaccgattggattaacaaagacggtagcaaggcttacaacgaagatggcacggtcaaacaatcgaccatcggtaagtacaacgagaaatacggtgacgcatccggtaactacgttttcatccgtgcccacgataacaacgtccaggacatcatcgccgagatcatcaagaaagagatcaacccgaaaagcgacggcttcaccatcaccgacgccgaaatgaagcaagcctttgaaatctataacaaagatatgctgtcgagcgacaaaaagtataccctgaataacattccggcagcgtatgccgtgatgttgcagaatatggaaacgattacccgcgtctattacggtgatctgtatacggacgacggtcactacatggaaaccaaatctccgtattacgataccatcgtgaatttgatgaagagccgtatcaagtatgtttcgggtggccaggcgcaacgtagctattggctgccgaccgacggtaagatggacaatagcgacgttgagctgtaccgcacgaatgaggtttacacgagcgtgcgctatggtaaggatatcatgaccgctaatgataccgaaggctctaagtattcccgcaccagcggccaagtcaccttggtcgcgaacaatccgaagctgaatctggaccaaagcgccaagttgaatgtggagatgggcaaaatccatgcgaatcagaagtatcgcgcactgattgtcggcactgcggacggcattaagaactttacttccgacgcggacgccattgcagcgggttatgtgaaagaaaccgatagcaacggcgtgctgaccttcggtgctaacgacattaagggctacgaaacgtttgatatgagcggtttcgtggcggtgtgggttccggtgggtgcatctgacaatcaggacattcgtgttgcgccgagcaccgaggcaaagaaagaaggtgagctgaccttgaaggcgacggaagcgtatgatagccagctgatttacgaaggctttagcaatttccagacgatcccagatggcagcgatccgtccgtgtatacgaaccgcaagattgcggagaacgtggatctgttcaaaagctggggtgtcaccagctttgagatggcaccgcaatttgtctcggcggatgatggcacctttctggatagcgttattcagaatggctacgccttcgccgaccgttatgacctggccatgtccaagaacaacaagtatggtagcaaagaggacctgcgtgatgcactgaaagcactgcataaggcgggtattcaagctatcgcagactgggttccagaccagatctaccagctgccgggcaaagaagttgtcaccgccacccgtacggatggtgctggccgtaagatcgcagacgcgattatcgaccattctctgtatgttgcaaacagcaaaagcagcggcaaagattatcaagcaaagtacggtggcgagttcctggccgagctgaaagccaaatacccggaaatgttcaaagttaacatgattagcacgggtaagccgattgatgactccgtgaaattgaagcaatggaaagccgagtacttcaatggcaccaacgttttggaacgtggtgtcggctatgttctgagcgacgaggcgaccggtaagtatttcacggtgaccaaagaaggcaatttcattccgctgcaactgacgggtaaagagaaagttatcacgggtttctccagcgatggtaagggtatcacctatttcggtacgagcggtacgcaggcgaagtctgcgtttgttaccttcaatggtaacacctactatttcgacgcgcgtggccacatggttaccaatagcgaatacagcccgaatggcaaggacgtctaccgttttctgccgaacggtatcatgctgagcaatgcgttttacattgatgcgaacggtaatacctacctgtacaactctaagggtcaaatgtacaaaggcggttacacgaaattcgatgtttctgaaacggataaggacggtaaagagtccaaggtcgtcaagttccgctactttacgaacgaaggcgtcatggccaagggtgttaccgtcattgatggttttacccaatacttcggtgaggacggctttcaagcgaaggataagctggtcaccttcaagggcaagacgtattacttcgacgcacacactggtaatggtatcaaagatacctggcgcaatatcaatggtaaatggtactatttcgacgcgaatggcgttgctgcgaccggtgcgcaggtgattaacggccagaaactgtacttcaacgaggatggctcccaagtcaaaggcggcgtggttaagaacgcagacggcacctatagcaaatacaaagaaggttttggtgagctggttactaacgagtttttcacgactgatggcaatgtttggtactacgccggtgcaaatggtaaaaccgttaccggtgcacaagtgatcaacggccaacatttgtacttcaatgcggacggttcccaggtgaagggtggcgttgtcaagaacgcggatggcacctacagcaagtacaatgctagcactggtgaacgtctgacgaacgagttctttacgaccggtgataacaattggtattacattggcgcaaacggtaagagcgtgacgggtgaggtcaagattggtgatgatacttactttttcgcgaaggatggcaaacaagttaaaggtcaaaccgtcagcgccggtaatggtcgcattagctactactacggtgacagcggcaagcgtgcggttagcacctggattgagattcagccgggtgtttatgtgtatttcgacaaaaacggtttggcgtaccctccgcgtgttctgaattaa

SEQ ID NO: 8 - protein, 1477 aa, Streptococcus salivarius
MDETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 9 - DNA, 4311 bp, Streptococcus downei
atggttgacggcaaatactactactacgatcaggacggcaacgtaaagaaaaacttcgcggttagcgtgggcgagaaaatctattactttgacgaaactggcgcctacaaagacaccagcaaagttgaggcggacaaaagcggcagcgacattagcaaggaagagactaccttcgcggcaaacaaccgcgcctacagcaccagcgcggagaattttgaggcgatcgacaattatctgaccgcggactcctggtatcgtcctaaatccatcctgaaggatggcaaaacgtggacggaaagcagcaaagatgactttcgtccgctgctgatggcgtggtggccggataccgaaacgaagcgcaattacgtgaactacatgaacaaagttgttggcatcgacaagacctataccgcggaaaccagccaggccgacttgaccgctgcggcggaactggtgcaagcacgcattgagcagaagatcacgaccgaacagaacacgaaatggctgcgtgaggcaatctcggcatttgttaaaacgcaaccgcagtggaacggtgaaagcgagaagccgtacgacgatcacctgcaaaacggtgctctgaaatttgataatcagagcgacctgaccccggatacgcaaagcaactaccgtctgttgaaccgtaccccgactaatcagacgggtagcctggacagccgcttcacttataacgcgaacgaccctttgggcggttatgagctgctgctggcaaatgacgtcgataacagcaatccgatcgtgcaggcggagcagctgaactggctgcattacctgctgaattttggtacgatctacgccaaagatgccgacgctaacttcgatagcattcgtgtggacgcggttgataacgtcgatgcggatctgctgcaaattagcagcgattacctgaaagcagcctacggcattgataagaataacaaaaacgcgaacaaccacgtgagcattgtcgaagcctggagcgataatgataccccgtacctgcatgacgatggtgacaacctgatgaatatggataacaaatttcgcctgtccatgctgtggtcgctggccaaaccgctggacaagcgtagcggtctgaacccgctgattcataacagcttggtggatcgtgaagttgatgaccgcgaggttgaaacggttccgagctattcttttgcacgtgcgcatgatagcgaggtccaggacttgatccgtgacatcatcaaggcagagatcaatccgaacgcattcggttatagctttacccaagacgagattgaccaggcctttaagatttacaatgaggatctgaagaaaacggataagaaatacacccactataatgtgccgttgagctacaccctgctgctgacgaataagggtagcatcccacgtgtctactatggtgatatgtttaccgacgatggtcagtatatggcgaacaaaaccgtcaactatgacgccattgaatctctgctgaaagcgcgtatgaagtatgtcgctggcggtcaagcaatgcagaactaccaaatcggtaatggtgagatcctgaccagcgttcgttatggtaagggtgccctgaaacagagcgacaaaggtgatgcgaccacgcgcaccagcggtgtcggtgtcgttatgggcaatcagccaaactttagcttggacggcaaagtggtggctctgaacatgggcgcagctcatgcgaatcaggagtatcgtgcgctgatggttagcacgaaagacggtgttgccacgtatgcgaccgatgcagatgcgagcaaagccggtctggtcaaacgtaccgacgaaaacggctacctgtatttcctgaatgacgacctgaagggtgtggccaatcctcaggtgagcggtttcttgcaggtgtgggttccggtgggtgccgcggatgatcaagatatccgtgttgcagctagcgataccgcatccaccgatggcaagagcctgcaccaagacgccgcgatggatagccgtgttatgtttgaaggcttctctaactttcagtcctttgccacgaaagaagaggaatataccaacgtcgttatcgccaacaatgtggataagttcgttagctggggtatcacggatttcgagatggccccacaatatgtttccagcaccgacggtcaattcctggactctgtcattcagaacggttatgcttttacggaccgttatgacttgggcatgtctaaggcaaacaaatacggcacggccgatcaactggttaaggccattaaggccctgcacgcgaagggcctgaaggttatggcagattgggtgccggatcagatgtataccttcccgaaacaggaagtcgtgaccgttacccgtaccgacaaatttggcaaaccgatcgcaggttcccaaatcaatcatagcctgtatgttaccgataccaagtccagcggcgatgactatcaggccaaatatggtggtgcgtttctggacgagctgaaggagaaatatccggagctgttcacgaagaaacaaatcagcacgggtcaagctattgacccgagcgtgaaaatcaaacagtggtctgctaagtatttcaatggctccaacatcctgggtcgcggtgcggactacgtactgtcggatcaggcgagcaacaaatacctgaacgtgtctgacgataaactgttcctgccgaaaaccttgctgggccaagttgtcgagagcggtatccgctttgacggcactggttatgtgtacaactctagcactacgggtgaaaaagttaccgattccttcattacggaggcaggtaatctgtactacttcggtcaagacggctatatggtgaccggcgcacagaacattaagggcagcaactattacttcctggccaatggtgcggccctgcgtaacaccgtttacaccgatgcgcaaggtcagaatcactattacggcaacgacggcaagcgttatgagaatggttaccaacagttcggcaacgattcttggcgttacttcaaaaatggcgtgatggcgctgggtctgactacggtggatggtcacgtgcagtatttcgataaagatggtgtccaggccaaggataagatcattgtcacccgcgatggcaaagtccgctatttcgaccagcacaacggtaatgcggttactaacacgttcgttgcggacaagacgggtcactggtactatctgggcaaagacggcgtcgcggttaccggtgcgcagactgtgggtaaacagcatttgtactttgaagcgaacggtcaacaagtcaagggtgacttcgtgacggctaaagacggtaaactgtacttctatgatgtggacagcggcgacatgtggaccaatacctttatcgaggataaagcgggtaattggttctacctgggtaaggacggtgcggccgtcaccggtgcacagacgatcaaaggccagaaattgtatttcaaagccaacggtcagcaagttaaaggtgacattgtcaaggacgcggacggtaagatccgttattacgacgctcagaccggtgaacaggtctttaacaagtccgttagcgtcaacggtaagacctactatttcggtagcgacggcaccgcgcaaacccaggcgaatccgaaaggccaaacctttaaggatggtagcggcgttctgcgtttctacaatttggagggccagtatgtctcgggcagcggctggtacgaaacggccgagcacgagtgggtatatgtgaaatccggtaaagttctgaccggtgcccagacgattggtaatcaacgtgtttacttcaaggacaatggtcaccaggtgaaaggccagctggtcacgggtaatgacggtaaattgcgttactacgacgcgaacagcggtgatcaagcattcaacaaatccgtcacggttaacggtaaaacctactactttggcagcgatggtacggcgcagacgcaggctaatcctaagggtcagaccttcaaagatggtagcggcgtgctgcgtttttacaacttggaaggccaatacgtgtctggcagcggttggtacaagaatgcgcagggccagtggctgtacgtgaaagatggcaaggtcctgaccggtctgcaaacggtcggcaatcagaaggtctacttcgacaaaaatggcatccaagcaaagggtaaggccgttcgcacgtccgatggtaaagtgcgctactttgatgagaatagcggtagcatgattacgaaccaatggaagttcgtttacggtcaatactattacttcggttctgacggcgcagcggtttaccgtggttggaactaa

SEQ ID NO: 10 - protein, 1436 aa, Streptococcus downei
MVDGKYYYYDQDGNVKKNFAVSVGEKIYYFDETGAYKDTSKVEADKSGSDISKEETTFAANNRAYSTSAENFEAIDNYLTADSWYRPKSILKDGKTWTESSKDDFRPLLMAWWPDTETKRNYVNYMNKVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYELLLANDVDNSNPIVQAEQLNWLHYLLNFGTIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDLIRDIIKAEINPNAFGYSFTQDEIDQAFKIYNEDLKKTDKKYTHYNVPLSYTLLLTNKGSIPRVYYGDMFTDDGQYMANKTVNYDAIESLLKARMKYVAGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQASNKYLNVSDDKLFLPKTLLGQVVESGIRFDGTGYVYNSSTTGEKVTDSFITEAGNLYYFGQDGYMVTGAQNIKGSNYYFLANGAALRNTVYTDAQGQNHYYGNDGKRYENGYQQFGNDSWRYFKNGVMALGLTTVDGHVQYFDKDGVQAKDKIIVTRDGKVRYFDQHNGNAVTNTFVADKTGHWYYLGKDGVAVTGAQTVGKQHLYFEANGQQVKGDFVTAKDGKLYFYDVDSGDMWTNTFIEDKAGNWFYLGKDGAAVTGAQTIKGQKLYFKANGQQVKGDIVKDADGKIRYYDAQTGEQVFNKSVSVNGKTYYFGSDGTAQTQANPKGQTFKDGSGVLRFYNLEGQYVSGSGWYETAEHEWVYVKSGKVLTGAQTIGNQRVYFKDNGHQVKGQLVTGNDGKLRYYDANSGDQAFNKSVTVNGKTYYFGSDGTAQTQANPKGQTFKDGSGVLRFYNLEGQYVSGSGWYKNAQGQWLYVKDGKVLTGLQTVGNQKVYFDKNGIQAKGKAVRTSDGKVRYFDENSGSMITNQWKFVYGQYYYFGSDGAAVYRGWN

SEQ ID NO: 11 - DNA, 3942 bp, Streptococcus mutans
atgattgacggcaaatactactactatgacaacaacggcaaagtacgcaccaatttcacgttgatcgcggacggtaaaatcctgcattttgatgaaactggcgcgtacaccgacactagcattgataccgtgaacaaggatattgtcacgacgcgtagcaacctgtataagaaatacaatcaagtgtatgatcgcagcgcgcagagcttcgagcatgttgatcactacctgacggcggaatcttggtaccgtccgaaatacattctgaaagatggcaagacctggacccagagcaccgagaaggacttccgtcctctgctgatgacctggtggccgagccaggaaacgcagcgccagtatgtcaacttcatgaacgcccagttgggtatcaacaaaacgtacgacgacaccagcaatcagctgcaattgaacatcgctgctgcaacgatccaagcaaagatcgaagccaaaatcacgacgctgaagaacaccgattggctgcgtcaaacgatcagcgcgttcgtcaaaacccaaagcgcttggaatagcgacagcgaaaagccgtttgatgaccatctgcaaaacggtgcggttctgtatgataacgaaggtaaattgacgccgtatgccaatagcaactatcgtattctgaaccgcacgccgaccaaccagaccggtaagaaggacccgcgttataccgccgacaacacgatcggcggctacgagtttctgctggccaacgacgtggataatagcaacccggtggttcaggccgagcagctgaactggctgcacttcctgatgaactttggtaatatctacgcaaacgaccctgacgctaacttcgactccatccgcgttgacgctgtcgataatgtggacgccgatctgttacagatcgcgggtgactatctgaaagcggcaaagggcatccataagaatgacaaagcggcgaacgaccacctgtccattctggaagcgtggagcgacaatgacactccgtatctgcatgatgatggcgacaacatgattaacatggataacaaactgcgcctgagcctgctgttctccctggcgaaaccgctgaatcagcgtagcggtatgaacccgttgattacgaacagcctggtcaaccgtactgatgataatgccgaaacggcggcagtgccaagctactcttttatccgtgcccacgatagcgaggtccaggatttgattcgtgatatcattaaggctgagattaacccgaacgtcgtcggttacagcttcacgatggaagagattaagaaggcatttgagatctacaataaggacctgttggccacggagaagaagtatacccactataacaccgcattgagctacgcgttgctgctgacgaacaagagcagcgtgccgcgtgtctactatggtgatatgtttacggacgatggtcaatacatggcccacaagaccattaactacgaggcaatcgaaaccctgctgaaagcacgtatcaagtacgtgtccggtggtcaggctatgcgcaaccagcaagtgggtaattcggagatcatcaccagcgtgcgttacggtaaaggtgcgctgaaggcgatggatacgggtgaccgcactacccgtacctctggtgtggcggtcattgagggcaacaacccgagcttgcgcctgaaggcttctgatcgtgtggttgtgaatatgggtgcggcccacaaaaatcaagcctatcgcccgctgctgttgacgaccgataacggcattaaggcctatcacagcgaccaagaagcggcaggcctggtgcgttacaccaacgaccgtggcgaactgatctttaccgcagccgacattaagggctacgcaaatccgcaagttagcggctacctgggcgtctgggtccctgttggcgcagcagctgatcaggacgttcgtgttgcggcgagcaccgcgccaagcacggacggcaagagcgttcaccagaacgcggctctggacagccgtgtgatgttcgagggtttctcgaacttccaggcatttgctaccaagaaagaagagtataccaatgtggtcatcgctaagaatgtggataagttcgcggagtggggtgtcaccgatttcgagatggctccgcaatacgtttctagcaccgacggtagctttttggatagcgtgattcaaaacggttatgcttttaccgaccgttacgacctgggcatcagcaagccgaacaaatatggcaccgcggacgatctggttaaagcgattaaggcattgcacagcaaaggcatcaaagttatggcggattgggttccggaccagatgtatgccctgccggaaaaagaggttgtgacggcaacccgtgttgacaaatacggtacgccggtagctggcagccagatcaaaaacacgctgtacgtggtcgatggtaaatctagcggtaaggaccagcaggcgaagtacggtggtgccttcctggaagagctgcaagcgaagtatccggaactgttcgcgcgcaaacagattagcaccggtgttccgatggacccgagcgtcaagattaagcaatggagcgcaaaatacttcaacggcacgaatatcctgggtcgtggtgctggttacgtgctgaaagatcaggcaaccaacacctactttaacatcagcgacaataaagagatcaatttcctgccaaagacgttgctgaaccaggattctcaagttggctttagctacgacggtaagggctatgtgtactacagcacctcgggctaccaggctaaaaacacgttcatcagcgagggtgacaagtggtattacttcgacaataacggttatatggttaccggcgcacagagcattaatggtgtgaactattacttcctgccgaatggtttacagctgcgtgatgcgattctgaaaaatgaggacggtacgtacgcgtattatggcaatgatggtcgccgctacgagaatggctattatcagtttatgagcggtgtttggcgccatttcaataatggcgagatgtccgttggtctgaccgtcattgacggtcaagttcaatactttgacgagatgggttaccaggcgaaaggcaaattcgttaccaccgcggatggtaagatccgttacttcgataagcagagcggcaatatgtatcgtaatcgtttcattgagaacgaagagggcaaatggctgtacctgggtgaggacggcgcggcagtcaccggtagccagacgatcaatggtcagcacctgtattttcgtgctaacggcgttcaggttaagggtgagttcgtgaccgatcgtcatggccgcatctcttattacgacggcaacagcggtgatcagatccgcaaccgtttcgtccgcaatgcgcaaggccagtggttttactttgacaacaatggctatgcagtaactggtgctcgtacgatcaacggccagcacctgtatttccgcgcgaacggtgttcaggtaaaaggtgagtttgttacggaccgccacggccgcattagctattatgatggtaatagcggtgaccaaattcgcaatcgtttcgtgcgtaatgcacagggtcagtggttctacttcgacaataatggttatgcagtcacgggtgcacgtaccattaacggccaacacctgtactttcgcgccaatggtgtgcaagtgaaaggcgaatttgttactgatcgttatggtcgtatcagctactatgatggcaattctggcgaccaaattcgcaatcgctttgttcgtaacgcccaaggtcaatggttctatttcgacaacaacggttacgcggtgaccggtgcccgcacgattaatggtcaacacttgtacttccgtgccaacggtgtccaggtgaagggtgaatttgtgaccgaccgctatggtcgcatttcttactacgacgcaaattccggtgaacgcgtccgtatcaattaa

SEQ ID NO: 12 - protein, 1313 aa, Streptococcus mutans
MIDGKYYYYDNNGKVRTNFTLIADGKILHFDETGAYTDTSIDTVNKDIVTTRSNLYKKYNQVYDRSAQSFEHVDHYLTAESWYRPKYILKDGKTWTQSTEKDFRPLLMTWWPSQETQRQYVNFMNAQLGINKTYDDTSNQLQLNIAAATIQAKIEAKITTLKNTDWLRQTISAFVKTQSAWNSDSEKPFDDHLQNGAVLYDNEGKLTPYANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKAMDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFNISDNKEINFLPKTLLNQDSQVGFSYDGKGYVYYSTSGYQAKNTFISEGDKWYYFDNNGYMVTGAQSINGVNYYFLPNGLQLRDAILKNEDGTYAYYGNDGRRYENGYYQFMSGVWRHFNNGEMSVGLTVIDGQVQYFDEMGYQAKGKFVTTADGKIRYFDKQSGNMYRNRFIENEEGKWLYLGEDGAAVTGSQTINGQHLYFRANGVQVKGEFVTDRHGRISYYDGNSGDQIRNRFVRNAQGQWFYFDNNGYAVTGARTINGQHLYFRANGVQVKGEFVTDRHGRISYYDGNSGDQIRNRFVRNAQGQWFYFDNNGYAVTGARTINGQHLYFRANGVQVKGEFVTDRYGRISYYDGNSGDQIRNRFVRNAQGQWFYFDNNGYAVTGARTINGQHLYFRANGVQVKGEFVTDRYGRISYYDANSGERVRIN

SEQ ID NO: 13 - DNA, 3972 bp, Streptococcus dentirousetti
atggttgacggcaaatactactactacgatgcagacggcaacgtaaagaaaaacttcgcggttagcgttggcgatgccattttctattttgatgaaacgggtgcctacaaagataccagcaaagttgatgcggataagaccagctctagcgtcaatcagaccacggaaacgttcgcagcgaataaccgtgcgtatagcaccgcagccgagaactttgaagcgattgataactacctgactgcggatagctggtatcgtccgaagtctatcttgaaagatggtacgacgtggaccgaaagcaccaaggatgattttcgcccgctgctgatggcgtggtggccggataccgaaaccaaacgtaactacgtgaactatatgaacaaggtggtcggtatcgacaaaacgtacaccgcggaaacgtcccaagctgacctgacggcggcagccgaactggtgcaggcgcgtatcgagcagaaaatcactagcgaaaagaatacgaagtggctgcgtgaggcgatttccgcgttcgttaagactcaaccgcagtggaatggcgagagcgagaaaccttatgatgaccacctgcaaaatggtgcgctgaagttcgacaatgaaaccagcctgaccccggatacgcagagcggctatcgcatcctgaaccgtaccccgacgaatcaaaccggtagcctggacccgcgcttcacctttaatcagaatgacccgctgggtggttatgagtatttgctggctaatgatgtcgataacagcaacccggtcgttcaggccgagagcctgaactggctgcattacctgctgaattttggtagcatttacgcgaatgatccggaggccaatttcgacagcatccgtgtggacgcggtggacaatgttgacgcagacctgctgcaaattagctcggattacctgaaatcggcgtacaaaattgacaagaacaacaaaaatgcgaacgaccacgttagcatcgtcgaggcgtggagcgacaatgataccccgtacctgaatgatgatggcgacaatctgatgaacatggataacaagtttcgtctgagcatgctgtggagcctggcgaagccaaccaatgtccgtagcggcttgaatccgctgatccacaacagcgtggttgaccgtgaggtggacgaccgtgaagttgaggctaccccgaattacagctttgcacgcgcacacgacagcgaagttcaagatttgattcgcgacatcatcaaagctgagatcaacccaaacagcttcggttatagctttacccaagaggaaatcgaccaggccttcaagatctacaatgaggatttgaagaaaaccaataagaagtatacccactacaacgtcccgctgagctacaccctgctgctgacgaacaagggcagcattccacgcatttactacggtgacatgtttacggatgacggtcagtatatggccaacaaaaccgttaactatgacgccattgagagcctgctgaaagcacgtatgaagtatgttagcggtggccaagcgatgcagaattacaacatcggcaacggcgagattctgaccagcgtccgttacggtaagggtgccctgaaacagagcgacaaaggcgataagactactcgtaccagcggtattggcgttgtgatgggtaaccagagcaatttcagcctggagggcaaggtggtggccctgaatatgggtgcaacgcataccaaacagaagtatcgtgcattgatggtgtctacggaaaccggcgtggcgatttacaatagcgatgaagaagcagaggcagcaggcctgatcaaaacgaccgatgagaatggttatttgtactttctgaatgacgatctgaagggcgtggctaacccgcaggtcagcggcttcctgcaagtgtgggttccggttggtgcaccggctgaccaggacattcgtgtggcggcgaccgatgcggcttctaccgacggtaagagcctgcatcaggacgcagctctggattctcgcgtcatgtttgaaggtttcagcaacttccagagcttcgcaaccaaggaagaggaatacaccaacgttgttattgcaaagaacgtggataagttcgtgagctggggtatcaccgacttcgagatggcaccgcagtacgttagctctaccgatggcacctttctggatagcgtgattcaaaatggctatgcctttacggaccgttacgacctgggtatgagcaaagcaaacaagtatggtactgctgaccaactggtggccgcgattaaagcgctgcatgcgaagggtctgcgtgtgatggcggattgggtcccagatcaaatgtacactttccctaagaaggaagtggttaccgttacccgtacggacaaatttggcaatccagtggcaggcagccaaatcaaccacaccttgtacgtcactgatactaagggtagcggtgacgactaccaggcgaagtacggtggcgcattcctggatgaactgaaagaaaagtacccggagctgtttaccaagaagcaaatcagcaccggtcaggcaatcgacccgagcgtgaaaatcaagcagtggagcgcgaagtacttcaacggtagcaatatcttgggtcgcggtgcgaactacgtgctgtccgaccaggcgtctaacaagtactttaacgtggccgaaggtaaagtctttctgccagcggcgatgctgggtaaggtcgtcgagagcggtatccgtttcgacggtaaaggttatatctataacagcagcaccactggcgaacaagtgaaggacagcttcattaccgaagcgggtaacttgtactattttggcaaagatggttatatggtcatgggtgcacagaatatccagggtgctaactactacttcttggcgaatggtgcggccctgcgcaatagcatcctgacggatcaggatggcaaaagccactattatgcaaatgacggcaagcgttatgagaacggctactatcaattcggtaacgactcctggcgctattttgaaaacggcgttatggccgttggtttgacgcgcgttgcgggccacgaccaatactttgataaggatggtatccaagcgaagaataagatcattgttacgcgtgacggtaaggtccgctacttcgacgaacacaacggcaatgctgccacgaatacgtttatcagcgatcaagccggccattggtactacctgggtaaagatggtgtcgccgtgacgggtgcgcagaccgttggcaagcaacacctgtacttcgaggctaacggccaacaagtaaaaggcgattttgttaccgccaaggacggtaagttgtattttctggacggtgactctggcgacatgtggaccgataccttcgtccaggataaggctggtcattggttctatctgggcaaagacggtgcggcggtaaccggtgcccagaccgtccgtggtcagaagctgtacttcaaagcgaatggccagcaggttaagggtgacattgtgaaaggcgcggatggtaaaatccgttactatgatgcaaattccggtgaccaggtttacaatcgcacggtgaaaggctccgacggcaagacctatatcattggtaatgacggcgtcgcaatcacgcaaaccatcgccaaaggccagaccatcaaggatggcagcgttctgcgcttctatagcatggagggtcagctggtgaccggcagcggctggtattccaacgcgaaaggtcaatggttgtatgtcaagaacggtcaagtcctgacgggtttgcagacggtgggcagccagcgtgtgtactttgacgcaaatggtattcaagcgaaaggtaaagcagtgcgtacctccgatggcaaactgcgttacttcgatgcgaacagcggcagcatgatcaccaatcagtggaaagaagttaatggtcagtactactatttcgacaacaacggtgttgcgatctatcgcggttggaactaa

SEQ ID NO: 14 - protein, 1323 aa, Streptococcus dentirousetti
MVDGKYYYYDADGNVKKNFAVSVGDAIFYFDETGAYKDTSKVDADKTSSSVNQTTETFAANNRAYSTAAENFEAIDNYLTADSWYRPKSILKDGTTWTESTKDDFRPLLMAWWPDTETKRNYVNYMNKVVGIDKTYTAETSQADLTAAAELVQARIEQKITSEKNTKWLREAISAFVKTQPQWNGESEKPYDDHLQNGALKFDNETSLTPDTQSGYRILNRTPTNQTGSLDPRFTFNQNDPLGGYEYLLANDVDNSNPVVQAESLNWLHYLLNFGSIYANDPEANFDSIRVDAVDNVDADLLQISSDYLKSAYKIDKNNKNANDHVSIVEAWSDNDTPYLNDDGDNLMNMDNKFRLSMLWSLAKPTNVRSGLNPLIHNSVVDREVDDREVEATPNYSFARAHDSEVQDLIRDIIKAEINPNSFGYSFTQEEIDQAFKIYNEDLKKTNKKYTHYNVPLSYTLLLTNKGSIPRIYYGDMFTDDGQYMANKTVNYDAIESLLKARMKYVSGGQAMQNYNIGNGEILTSVRYGKGALKQSDKGDKTTRTSGIGVVMGNQSNFSLEGKVVALNMGATHTKQKYRALMVSTETGVAIYNSDEEAEAAGLIKTTDENGYLYFLNDDLKGVANPQVSGFLQVWVPVGAPADQDIRVAATDAASTDGKSLHQDAALDSRVMFEGFSNFQSFATKEEEYTNVVIAKNVDKFVSWGITDFEMAPQYVSSTDGTFLDSVIQNGYAFTDRYDLGMSKANKYGTADQLVAAIKALHAKGLRVMADWVPDQMYTFPKKEVVTVTRTDKFGNPVAGSQINHTLYVTDTKGSGDDYQAKYGGAFLDELKEKYPELFTKKQISTGQAIDPSVKIKQWSAKYFNGSNILGRGANYVLSDQASNKYFNVAEGKVFLPAAMLGKVVESGIRFDGKGYIYNSSTTGEQVKDSFITEAGNLYYFGKDGYMVMGAQNIQGANYYFLANGAALRNSILTDQDGKSHYYANDGKRYENGYYQFGNDSWRYFENGVMAVGLTRVAGHDQYFDKDGIQAKNKIIVTRDGKVRYFDEHNGNAATNTFISDQAGHWYYLGKDGVAVTGAQTVGKQHLYFEANGQQVKGDFVTAKDGKLYFLDGDSGDMWTDTFVQDKAGHWFYLGKDGAAVTGAQTVRGQKLYFKANGQQVKGDIVKGADGKIRYYDANSGDQVYNRTVKGSDGKTYIIGNDGVAITQTIAKGQTIKDGSVLRFYSMEGQLVTGSGWYSNAKGQWLYVKNGQVLTGLQTVGSQRVYFDANGIQAKGKAVRTSDGKLRYFDANSGSMITNQWKEVNGQYYYFDNNGVAIYRGWN

SEQ ID NO: 15 - DNA, 4047 bp, Streptococcus oralis
atgatcgacggcaaaaactactacgtacaggatgatggcacggtaaagaagaatttcgcggtagaactgaatggtcgtatcctgtattttgatgcagaaaccggcgctctggttgatagcaacgagtatcagttccaacagggtacgagcagcctgaacaatgaattttctcagaagaacgcattctatggtacgaccgataaggatattgagactgtggatggctacctgaccgcagatagctggtatcgcccgaaattcatcctgaaggatggcaagacgtggaccgcgagcacggaaacggatctgcgtccgctgttgatggcatggtggccggacaagcgtacccaaatcaactatctgaactacatgaaccagcagggtctgggtgcgggtgcgtttgagaacaaagtggagcaggccctgctgacgggtgcaagccaacaggtacaacgcaagatcgaagagaagattggtaaagagggtgataccaagtggctgcgcaccctgatgggtgcgttcgtgaaaacgcaaccaaactggaatatcaaaaccgagtctgaaacgaccggcacgaaaaaggaccatctgcaaggcggtgcactgctgtatacgaacaacgagaaatccccgcacgcggacagcaaatttcgtctgctgaatcgtaccccgaccagccaaaccggcacgccgaagtatttcatcgacaagtctaacggtggctacgaatttctgctggcgaacgattttgacaatagcaatcctgcggtacaagctgagcagctgaattggctgcactacatgatgaactttggcagcattgttgcgaatgatccgaccgcgaatttcgacggcgttcgtgtggatgctgttgataacgtcaatgcggacttgttgcaaattgcaagcgattactttaagagccgttacaaagtcggtgagagcgaagaagaagcgatcaagcacctgtccatcctggaagcatggagcgataacgacccggactacaacaaagataccaagggtgcacagttggcgattgataacaaactgcgcctgagcctgctgtactctttcatgcgtaatctgagcatccgtagcggtgttgaaccgacgattaccaatagcctgaatgaccgttccagcgaaaagaagaacggcgagcgtatggcaaattacatcttcgtgcgtgcccacgatagcgaggtccaaacggtgatcgccgacatcattcgcgaaaacatcaatccgaacaccgacggcctgacgtttacgatggacgagctgaagcaggcattcaagatttacaacgaggacatgcgcaaggcggacaaaaagtatacccagtttaacattcctaccgcacacgcgctgatgctgtctaataaggattctattacccgcgtgtactatggtgatctgtatactgacgatggtcagtacatggagaagaaaagcccgtatcacgatgcgattgacgctctgctgcgtgcacgtattaaatacgtcgcgggtggccaggatatgaaagtgacctatatgggcgtgccgcgtgaagcggataagtggagctataacggcattctgaccagcgtgcgctatggcacgggcgctaacgaagccacggatgagggcactgcggaaacgcgcacgcaaggtatggcagtgattgcgagcaataatccaaatctgaaactgaatgaatgggacaagttgcaagtcaacatgggtgcggcgcataagaatcaatattaccgtccggttctgctgaccactaaggacggtatcagccgttatctgaccgatgaagaagtgcctcagagcctgtggaaaaagacggacgcaaacggtattctgaccttcgacatgaatgatattgctggctacagcaacgtgcaagttagcggttacctggccgtctgggtcccggtcggtgcgaaggcggatcaagatgcgcgcacgaccgcatccaagaagaaaaatgcgtcgggtcaggtgtacgaaagcagcgcggctctggatagccagctgatttacgaaggtttcagcaactttcaagactttgccactcgcgatgatcagtacacgaacaaggtcattgcgaaaaacgtgaatctgttcaaagaatggggtgtgaccagcttcgagctgccgccgcagtacgtgagcagccaagatggcacctttctggacagcattatccaaaacggctatgcatttgaagaccgttacgatatggcgatgagcaagaataacaagtatggtagcctgaaagacctgttgaacgcgctgcgcgcactgcacagcgtcaacattcaagcaatcgccgattgggtgccggaccaaatttacaacttgccgggcaaagaggtggtgaccgcaactcgtgtcaacaactacggcacctaccgtgagggtgctgaaatcaaagaaaagctgtatgtcgccaatagcaagaccaacgaaaccgatttccaaggtaaatacggtggtgcgttcctggatgagctgaaggcgaagtacccggagattttcgagcgtgtccaaatcagcaacggccaaaagatgactaccgatgaaaagatcaccaaatggagcgcgaaatactttaatggcaccaatattctgggtcgtggcgcgtactatgtcctgaaagattgggccagcaatgattacctgacgaaccgtaacggcgagattgttttgccgaagcaactggttaacaagaatagctataccggctttgtcagcgacgcgaacggcacgaagttctattctacctctggctaccaggcgaagaacagcttcattcaagacgaaaacggtaattggtattactttgacaaacgtggttatctggttacgggcgcacacgagattgatggcaagcatgtctacttcctgaaaaacggtatccaactgcgtgacagcatccgtgaggatgagaacggtaatcaatactattacgaccagaccggcgcacaagtgctgaaccgttactacacgacggacggtcagaattggcgctatttcgatgcgaaaggtgttatggcacgcggcctggtaaagattggtgacggccaacagtttttcgatgaaaacggttaccaggtcaagggcaagattgttagcgcaaaagacggcaagctgcgctactttgataaagactctggcaatgctgtcattaatcgtttcgcgcagggtgacaatccgagcgactggtactatttcggtgtggaatttgctaaactgacgggtttgcaaaagatcggccagcagacgctgtattttgaccaagacggtaagcaagtcaaaggtaagatcgtaactctgtcggacaaaagcattcgttacttcgatgccaacagcggtgaaatggcggttggcaagttcgcggaaggtgcaaagaatgagtggtattatttcgataaaaccggcaaagcggttactggtttgcagaaaattggtaagcagaccctgtactttgaccaggacggtaaacaggttaaaggcaaggttgtcacgctggctgataaaagcatccgctacttcgacgcagactccggcgagatggcggtcggtaagtttgcagagggtgcgaagaacgagtggtactattttgatcagactggcaaggccgtgactggtttgcaaaagattgacaagcaaaccttgtacttcgaccaggacggtaaacaagtcaagggtaagattgtgacgttgagcgacaagtcgatccgttactttgatgctaatagcggtgagatggctactaacaaattcgtcgagggctcgcagaatgaatggtactacttcgatcaagcgggtaaggctgttacgggcttgcaacaggtcggtcagcaaactctgtacttcacccaggatggtaagcaagtgaagggtaaggtcgtggacgtgaacggtgtttctcgttatttcgacgcaaactccggtgacatggctcgttctaaatggattcaactggaagatggcagctggatgtatttcgaccgtgacggtcgtggccagaattttggccgtaactaa

SEQ ID NO: 16 - protein, 1348 aa, Streptococcus oralis
MIDGKNYYVQDDGTVKKNFAVELNGRILYFDAETGALVDSNEYQFQQGTSSLNNEFSQKNAFYGTTDKDIETVDGYLTADSWYRPKFILKDGKTWTASTETDLRPLLMAWWPDKRTQINYLNYMNQQGLGAGAFENKVEQALLTGASQQVQRKIEEKIGKEGDTKWLRTLMGAFVKTQPNWNIKTESETTGTKKDHLQGGALLYTNNEKSPHADSKFRLLNRTPTSQTGTPKYFIDKSNGGYEFLLANDFDNSNPAVQAEQLNWLHYMMNFGSIVANDPTANFDGVRVDAVDNVNADLLQIASDYFKSRYKVGESEEEAIKHLSILEAWSDNDPDYNKDTKGAQLAIDNKLRLSLLYSFMRNLSIRSGVEPTITNSLNDRSSEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARTTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLKDLLNALRALHSVNIQAIADWVPDQIYNLPGKEVVTATRVNNYGTYREGAEIKEKLYVANSKTNETDFQGKYGGAFLDELKAKYPEIFERVQISNGQKMTTDEKITKWSAKYFNGTNILGRGAYYVLKDWASNDYLTNRNGEIVLPKQLVNKNSYTGFVSDANGTKFYSTSGYQAKNSFIQDENGNWYYFDKRGYLVTGAHEIDGKHVYFLKNGIQLRDSIREDENGNQYYYDQTGAQVLNRYYTTDGQNWRYFDAKGVMARGLVKIGDGQQFFDENGYQVKGKIVSAKDGKLRYFDKDSGNAVINRFAQGDNPSDWYYFGVEFAKLTGLQKIGQQTLYFDQDGKQVKGKIVTLSDKSIRYFDANSGEMAVGKFAEGAKNEWYYFDKTGKAVTGLQKIGKQTLYFDQDGKQVKGKVVTLADKSIRYFDADSGEMAVGKFAEGAKNEWYYFDQTGKAVTGLQKIDKQTLYFDQDGKQVKGKIVTLSDKSIRYFDANSGEMATNKFVEGSQNEWYYFDQAGKAVTGLQQVGQQTLYFTQDGKQVKGKVVDVNGVSRYFDANSGDMARSKWIQLEDGSWMYFDRDGRGQNFGRN

SEQ ID NO: 17 - DNA, 4047 bp, Streptococcus sanguinis
atgattgatggtaaaaagtattacgtacaggacgacggcacggttaagaagaatttcgcggttgagctgaatggcaagatcctgtacttcgatgcagagactggtgcgttgattgacagcgcggagtatcaattccaacaaggcaccagcagcctgaataatgagttcactcaaaagaacgccttttacggtacgaccgataaggatgtggaaaccattgatggttacttgaccgccgattcctggtatcgtccgaagttcattctgaaagatggcaaaacctggacggcgagcacggaaattgacttgcgtccgttgttgatggcgtggtggccggacaaacagacccaggttagctacctgaattacatgaaccagcaaggcttgggtgcaggcgccttcgaaaacaaagtagagcaggcaattctgaccggtgcgtcccaacaggtacaacgtaaaatcgaagaacgcatcggtaaagagggtgataccaagtggctgcgtaccctgatgggtgcatttgtaaagacccagccgaactggaacattaagaccgagtccgaaaccactggcacgaataaagatcatctgcaaggtggcgcactgctgtatagcaattccgacaagacgagccatgccaactctaagtaccgtatcctgaaccgcaccccgaccaaccaaacgggcacgccgaaatactttattgacaagagcaatggtggttatgaatttctgctggcgaatgactttgacaatagcaatccggcagtgcaagcggaacagctgaactggttgcactttatgatgaattttggctccatcgttgcaaatgatccgacggccaacttcgacggcgtccgcgttgacgctgtggataacgtgaatgcggatctgttgcaaattgcgagcgactatttcaagagccgctataaagtcggcgaaagcgaagaagaggccattaagcacctgtccatcctggaagcgtggagcgacaacgacccggactacaacaaggatactaaaggtgcccaactgccgatcgacaacaaactgcgtctgagcctgctgtactccttcatgcgtaagctgagcatccgtagcggcgtcgagccgaccatcaccaactctctgaatgatcgcagcacggagaagaagaatggtgagcgtatggcaaactatatcttcgttcgtgcacatgatagcgaggtgcaaacggtcatcgccgacattatccgtgagaacatcaatccgaataccgacggcctgacgttcacgatggatgaactgaagcaggcctttaaaatttacaatgaggatatgcgtaaagccgacaaaaagtacacgcagttcaatatcccgaccgcgcacgcgctgatgctgagcaacaaagattctatcacccgcgtttactacggtgacctgtatacggatgacggtcagtatatggaaaagaaaagcccgtatcacgacgccattgacgctctgctgcgtgcgcgtatcaaatatgttgcgggtggtcaggacatgaaggtgacctatatgggcgtgccgcgtgaggcagataaatggagctataacggcatcctgaccagcgttcgttatggtacgggtgccaacgaggcaaccgacgagggtacggcagaaacccgtacccagggcatggccgtcattgccagcaacaatccgaacctgaaactgaacgagtgggacaagttgcaggtcaacatgggtgcagctcacaaaaaccaatactatcgtccggtgctgctgaccaccaaggacggcatctcgcgctacctgaccgacgaagaagtcccgcagagcctgtggaaaaagaccgatgcgaacggcatcttgacgtttgacatgaatgatattgcgggttacagcaacgtccaagtgagcggttatctggccgtctgggttcctgtgggtgcgaaggcggaccaggacgctcgtgttacggcatctaagaagaaaaatgcctctggccaagtttacgaaagcagcgcagccctggactcccagctgatctatgagggcttcagcaattttcaggactttgccacccgtgacgaccagtacactaacaaggttatcgcgaaaaacgtcaatctgtttaaagagtggggcgtcaccagcttcgaattgccgccacagtatgtgagcagccaagacggtacgttcctggatagcatcatccagaatggttatgcattcgaagatcgctatgatatggcgatgagcaaaaacaataagtacggtagcttgaacgacctgttgaacgccttgcgtgcactgcatagcgtgaatatccaagcgattgcggattgggtgccggaccagatttacaatctgccgggtaaagaagttgtcactgcaacccgtgttaacaattatggcacgtatcgtgagggtagcgagattaaagagaacctgtacgttgctaacaccaaaaccaatggtacggactaccaaggtaagtatggtggtgcgttcttggacgagctgaaagccaaataccctgagatttttgagcgcgtccaaatcagcaacggccagaagatgaccaccgacgagaagattacgaaatggtccgccaaacactttaacggcacgaacattctgggtcgtggtgcgtattatgtgctgaaagactgggcgagcaacgagtacctgaataacaaaaatggcgagatggttctgccgaagcagctggttaataaaaatgcatataccggcttcgtcagcgacgcgagcggcaccaaatactattctaccagcggctatcaggctcgtaatagctttattcaagatgaaaatggtaattggtactacttcaataaccgtggttatttggtgacgggtgcacaggaaatcgacggtaagcaactgtatttcctgaaaaacggcattcagctgcgtgattctctgcgtgaggacgaaaacggcaaccagtattactatgataagacgggtgcgcaagttctgaatcgttattacactacggacggccaaaattggcgctacttcgacgttaaaggcgtcatggcccgtggtctggtcacgatgggtggtaaccaacaattctttgaccaaaacggttaccaggttaaaggcaaaattgcgcgtgcaaaagacggtaaactgcgttacttcgataaagacagcggtaatgcggcagctaaccgtttcgcccaaggcgataaccctagcgactggtactatttcggtgcagatggtgttgcggttacgggcctgcaaaaggttggtcagcaaactctgtactttgatcaggacggcaagcaggtgaaaggtaaagttgttaccttggcggacaaaagcattcgttatttcgatgcaaacagcggcgagatggcggtgaacaagtttgtggaaggtgctaagaacgtgtggtactacttcgatcaagcaggcaaagcggtgaccggcctgcaaaccatcaataaacaagtgctgtatttcgaccaggatggtaaacaagtcaaaggtaaggtggtcacgctggctgataagtctatccgctacttcgacgcgaacagcggtgagatggcagtgggcaaattcgccgaaggcgcaaagaatgagtggtattactttgaccaggcgggcaaggctgttaccggtctgcaaaagatcggccaacagacgctgtatttcgaccagaacggtaaacaggttaagggtaaagtggtcaccctggcggataagagcatccgctatttcgacgctaactctggcgaaatggcaagcaataagttcgttgagggtgccaaaaatgaatggtactatttcgatcaggctggcaaggcagtgacgggtctgcaacaaattggccagcagaccctgtattttgaccagaatggcaaacaggtgaagggtaagattgtgtatgttaatggtgcgaatcgctactttgatgccaatagcggtgaaatggcgcgtaacaagtggattcagctggaagatggcagctggatgtattttgaccgcaatggtcgtggtcgtcgtttcggttggaactaa

SEQ ID NO: 18 - protein, 1348 aa, Streptococcus sanguinis
MIDGKKYYVQDDGTVKKNFAVELNGKILYFDAETGALIDSAEYQFQQGTSSLNNEFTQKNAFYGTTDKDVETIDGYLTADSWYRPKFILKDGKTWTASTEIDLRPLLMAWWPDKQTQVSYLNYMNQQGLGAGAFENKVEQAILTGASQQVQRKIEERIGKEGDTKWLRTLMGAFVKTQPNWNIKTESETTGTNKDHLQGGALLYSNSDKTSHANSKYRILNRTPTNQTGTPKYFIDKSNGGYEFLLANDFDNSNPAVQAEQLNWLHFMMNFGSIVANDPTANFDGVRVDAVDNVNADLLQIASDYFKSRYKVGESEEEAIKHLSILEAWSDNDPDYNKDTKGAQLPIDNKLRLSLLYSFMRKLSIRSGVEPTITNSLNDRSTEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARVTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLNDLLNALRALHSVNIQAIADWVPDQIYNLPGKEVVTATRVNNYGTYREGSEIKENLYVANTKTNGTDYQGKYGGAFLDELKAKYPEIFERVQISNGQKMTTDEKITKWSAKHFNGTNILGRGAYYVLKDWASNEYLNNKNGEMVLPKQLVNKNAYTGFVSDASGTKYYSTSGYQARNSFIQDENGNWYYFNNRGYLVTGAQEIDGKQLYFLKNGIQLRDSLREDENGNQYYYDKTGAQVLNRYYTTDGQNWRYFDVKGVMARGLVTMGGNQQFFDQNGYQVKGKIARAKDGKLRYFDKDSGNAAANRFAQGDNPSDWYYFGADGVAVTGLQKVGQQTLYFDQDGKQVKGKVVTLADKSIRYFDANSGEMAVNKFVEGAKNVWYYFDQAGKAVTGLQTINKQVLYFDQDGKQVKGKVVTLADKSIRYFDANSGEMAVGKFAEGAKNEWYYFDQAGKAVTGLQKIGQQTLYFDQNGKQVKGKVVTLADKSIRYFDANSGEMASNKFVEGAKNEWYYFDQAGKAVTGLQQIGQQTLYFDQNGKQVKGKIVYVNGANRYFDANSGEMARNKWIQLEDGSWMYFDRNGRGRRFGWN

SEQ ID NO: 19 - DNA, 4023 bp, unknown
  unknown Streptococcus sp. C150
atgatcgacggcaaatactactacgtaaacgaggacggcagccacaaagagaatttcgcgatcacggttaatggtcaactgctgtattttggtaaggatggcgcgctgaccagcagcagcacgtacagcttcacccaaggcactaccaatattgtggacggttttagcattaacaaccgtgcgtatgactccagcgaggcctctttcgagctgattgacggttatctgactgcggactcttggtaccgtccggcgagcattatcaaagacggtgtgacgtggcaagcatccaccgccgaggacttccgcccgttgctgatggcgtggtggccgaacgttgatactcaggtgaactacctgaactacatgtccaaagtctttaatctggatgctaaatacagctcgactgataaacaggaaaccctgaaggtggcggcgaaagatatccagatcaaaattgaacaaaagattcaggcggaaaagtccacgcaatggctgcgtgaaacgatcagcgcctttgtaaaaacccagccgcaatggaacaaagagactgagaactacagcaagggcggtggtgaggaccatctgcaaggtggtgccctgctgtatgttaatgactctcgtaccccgtgggcgaacagcaactatcgtttgctgaaccgcacggcgaccaaccagaccggtacgatcgacaagagcatcctggacgagcagagcgatccgaatcacatgggtggttttgatttcttgctggctaatgacgttgacttgagcaatccggtcgtccaggcggaacaactgaatcagatccactacctgatgaattggggttctattgtcatgggtgataaagacgcgaattttgacggtattcgtgtagacgcggtggataatgttgatgcggacatgctgcaattgtacaccaactatttccgcgaatactatggtgtcaacaaaagcgaggcaaacgcgctggcgcacattagcgtcctggaagcctggagcctgaatgacaaccattacaatgataagactgatgttgcggcgctggcaatggagaataagcagcgcttggcactgttgtttagcctggcgaaaccgattaaagaacgcacgcctgccgtgtctccgctgtacaacaatacgtttaacaccactcagcgtgatgaaaagacggactggatcaataaagatggttcgaaagcctacaatgaggatggcactgtcaagaaaagcaccatcggcaagtataacgagaagtatggtgatgctagcggcaactacgttttcatccgcgctcacgacaataacgtgcaagacatcatcgcggagatcattaagaaagagattaacgagaaatctgacggttttaccattacggattcggagatgaagcgtgcatttgagatctataacaaagacatgctgtctaatgacaaaaagtacacgctgaataacatcccggcggcgtacgcggttatgctgcaaaacatggaaacgattacccgcgtgtattacggcgatctgtacacggacgacggtaattacatggaagcgaaaagcccgtactacgatacgattgttaacttgatgaagtctcgcatcaaatacgtgagcggtggccaggcgcagcgcagctactggctgccgaccgatggtaagatggataagtcggatgttgagctgtaccgtacgaacgaagtgtacacgagcgtccgttacggcaaagacattatgaccgccgatgacacgcaaggtagcaaatacagccgtaccagcggtcaggtgaccctggtcgtcaacaacccaaaactgaccttggaccaaagcgcaaagctgaacgtggttatgggcaagattcatgctaatcagaagtaccgcgcactgattgtcggtaccccgaacggtattaagaatttcaccagcgacgcagaggctattgccgcaggctatgtcaaagaaaccgatggcaatggcgtgctgaccttcggtgcaaacgacatcaagggttatgaaactttcgatatgagcggcttcgtcgctgtttgggttccggtcggtgcgagcgacgaccaagatattcgtgtggcggcgtctacggcagcaaagaaagagggtgagctgacgctgaaagcgaccgaagcctatgactcccaactgatctatgaaggctttagcaatttccagaccatcccagatggcagcgatccttctgtttataccaatcgtaagatcgcggaaaatgttgatttgttcaagagctggggtgtcacgagcttcgaaatggctccgcagttcgtttctgcggacgatggcacgtttctggacagcgtcattcaaaacggctatgcgttcgcagaccgttatgatctggccatgagcaaaaacaataagtacggtagcaaagaagatctgcgtaacgcgctgaaggcactgcacaaagcaggcattcaggcgattgcagattgggtgccagaccaaatctaccagctgcctggcaaagaagttgttactgccacccgcacggacggtgctggtcgcaaaatcagcgatgcaatcatcgatcattccctgtacgttgcgaactccaagagctccggtaaggactaccaagcgaagtacggtggcgagttcttggcggaactgaaggcgaaatacccggaaatgttcaaagtgaacatgattagcaccggcaaaccgattgatgatagcgtgaaactgaagcagtggaaagcagaatacttcaacggcaccaatgtgctggatcgcggtgtcggttatgttctgagcgatgaggcaaccggtaagtatttcaccgttaccaaagagggtaactttatcccgttgcagctgaagggtaacaagaaggtgattaccggcttttccagcgacggtaagggcattacctatttcggtactagcggtaaccaagctaaatccgcgttcgtcacttttaacggtaacacgtactacttcgacgcacgtggccacatggttaccaacggtgagtactcgccgaatggtaaagatgtgtatcgttttctgccgaacggcattatgctgagcaacgcgttctatgttgacggcaatggcaacacctacctgtacaactccaaaggccaaatgtataaaggtggctatagcaaatttgacgtcacggaaacgaaggacggtaaagagagcaaagttgtcaagttccgctactttacgaacgagggcgtgatggcgaaaggtgtcacggttgtggatggcttcactcagtactttaacgaggatggcattcaaagcaaagacgagctggtcacttacaatggcaagacctattacttcgaagcacacacgggcaatgccattaagaatacgtggcgtaatatcaagggcaaatggtaccattttgatgctaacggtgtcgcggctactggcgcacaggttatcaacggtcagcacctgtacttcaatgaagatggctctcaagtaaaaggtagcatcgtcaaaaacgctgatggtacgttcagcaagtacaaggacagctctggcgatctggtggtgaacgagtttttcacgacgggtgataacgtctggtactatgctggtgccaatggcaaaacggttactggtgcacaggtgattaatggccagcacttgttcttcaaagaggatggcagccaggtcaagggcgactttgtgaagaatagcgacggcacctactccaagtatgacgctgcgagcggcgaacgtctgaccaacgagttcttcactacgggcgacaatcattggtactatattggcgccaacggtaagaccgttaccggtgaagttaagattggtgacgacacgtatttcttcgcaaaagacggtaagcaactgaaaggtcaaatcgttaccacccgtagcggtcgtatcagctactactttggtgatagcggtaagaaggctattagcacgtgggtggagatccagccgggtgtgtttgttttcttcgacaaaaacggcctggcttacccaccggagaatatgaactga

SEQ ID NO: 20 - protein, 1340 aa, unknown
  unknown Streptococcus sp. C150
MIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 21-24 - skipped (4 entries)

SEQ ID NO: 25 - DNA, 4308 bp, Streptococcus sobrinus
atggttgacggcaaatactactattatgatcaggatggcaacgttaagaagaatttcgcggttagcgttggtgacaagatctactactttgacgagactggtgcctacaaagacacctctaaagtggacgcggacaagtctagcagcgccgttagccaaaatgcgacgatctttgcggctaacaatcgtgcgtatagcacctctgctgagaactttgaggccgttgataactatctgacggcagatagctggtatcgtcctaaatctattctgaaagatggcaagacgtggaccgagtcgggtaaggacgacttccgtccgctgctgatggcgtggtggccggacacggagactaaacgcaattacgtgaattacatgaacctggttgtcggcatcgacaagacgtacaccgcggaaacctctcaagcagatttgaccgcagcggcggagctggtccaggcgcgtattgaacagaaaatcaccacggaacagaatacgaaatggctgcgcgaggcgatctctgctttcgtcaagacccagccgcagtggaatggtgaaagcgagaagccgtatgacgaccacctgcaaaacggtgctctgaaattcgataatcagagcgacctgaccccggacacccagagcaactatcgcctgctgaatcgcaccccgactaaccagactggcagcctggacagccgtttcacctataatgcgaacgatccgttgggtggctacgaatttctgctggctaacgacgtggataatagcaaccctgtggtgcaggcagaacaactgaactggttgcattacctgttgaattttggtagcatttacgcgaaagatgcggatgcaaacttcgattccatccgtgtggacgccgtggacaacgtcgatgcagatctgttgcagattagcagcgattacctgaaggcagcctatggcattgacaagaacaataagaacgcgaacaaccatgttagcattgttgaggcttggagcgataacgatacgccgtacctgcacgatgacggtgataacctgatgaacatggacaataagttccgcttgagcatgctgtggagcctggccaagccgctggacaagcgcagcggtctgaatcctctgattcataacagcctggtggaccgtgaggttgatgaccgtgaagtggaaacggttccgagctactcttttgcgcgtgcgcatgattccgaggtccaagacattatccgcgacattatcaaggccgaaatcaacccgaatagctttggttatagcttcacccaagaagagattgaccaggcgtttaagatctataatgaagatctgaagaaaaccgacaagaaatacacccactataatgtcccgttgagctatactttgctgctgacgaataaaggttcgattccgcgtgtgtattacggtgatatgttcaccgatgatggtcaatacatggcgaacaaaacggttaactatgatgccattgagtcgctgctgaaagcgcgcatgaagtacgttagcggcggtcaagcgatgcaaaactatcaaatcggcaatggtgagattctgaccagcgttcgttatggtaagggtgcattgaagcaatccgacaagggtgacgcgaccacgcgtacgtccggtgtgggcgtcgtgatgggcaaccagccgaactttagcctggacggcaaggtggtggcattgaacatgggtgccgctcatgcaaatcaggagtatcgtgcgctgatggtgagcaccaaggatggcgttgccacgtatgccaccgacgcggacgcaagcaaggcaggtctggtcaaacgcaccgatgaaaatggttatttgtactttctgaacgacgatctgaagggtgtggcaaacccacaagtcagcggtttcttgcaggtgtgggtcccagtgggtgcggctgacgatcaggacattcgtgttgcagcgagcgacacggctagcacggacggtaagtccctgcatcaagatgcggcaatggatagccgtgttatgtttgagggttttagcaacttccagagctttgcaaccaaagaagaagagtacaccaacgtagttattgcgaacaacgtggacaaattcgttagctggggtattaccgactttgagatggcaccgcaatatgtcagctccaccgatggccagtttctggatagcgttatccagaatggttacgcgttcaccgaccgttatgatctgggtatgagcaaagccaacaaatacggtaccgcggatcagctggttaaagcaatcaaagcgttgcacgcgaagggtctgaaggtgatggcggactgggttccagaccagatgtacacgtttccgaagcaggaagttgtcactgtcacgcgcaccgacaaatttggtaagccgattgcgggcagccaaatcaatcacagcctgtacgtgacggacaccaaatccagcggtgatgattaccaggccaaatatggtggtgcgttcctggatgagctgaaagagaaatacccggagctgttcaccaaaaagcagatctcgaccggtcaggcgatcgacccgagcgtgaagattaagcagtggagcgcgaaatactttaatggtagcaacattctgggtcgtggtgccgactacgtcctgtccgatcaagttagcaacaagtatttcaatgtggccagcgacacgctgtttctgccgtctagcctgttgggtaaggttgtcgaaagcggtattcgttacgatggcaaaggttatatctataacagcagcgcgactggcgaccaagtcaaggcgtcttttatcacggaagcaggcaatctgtactacttcggcaaagacggttacatggttactggtgcgcagaccattaacggtgcgaattacttcttcttggaaaatggtacggccctgcgtaataccatctacaccgatgcacagggcaactcccactattatgctaatgatggcaagcgttacgagaacggttaccagcagttcggcaacgattggcgttacttcaaagatggtaacatggccgtcggtctgaccacggtggatggtaacgttcagtatttcgacaaggacggtgtccaagctaaagacaagattattgtgacccgcgatggtaaggtgcgctactttgatcaacacaatggcaacgcggtcacgaatacctttatcgccgacaagaccggtcactggtactacctgggcaaagatggcgtcgcggtcaccggcgctcaaaccgtcggtaagcaaaaactgtattttgaggcgaacggtgagcaggtgaaaggcgactttgtgactagccatgaaggcaaactgtacttttatgatgttgacagcggcgacatgtggaccgataccttcatcgaggataaggccggcaactggttctacctgggtaaagacggcgcagcagttagcggtgcacagaccattcgcggtcaaaagctgtacttcaaggcgtacggtcaacaggtcaaaggtgacatcgttaaaggcaccgacggcaagatccgttactacgatgcgaaatccggcgagcaggttttcaataagacggtcaaagccgctgatggcaaaacctatgtgatcggcaacaatggtgtggcggtcgatccgagcgttgttaagggtcagacgttcaaagacgccagcggcgcactgcgtttttacaatctgaaaggtcaactggttacgggctccggttggtatgaaacggccaatcacgattgggtgtatattcagagcggtaaagcactgaccggtgagcaaaccatcaatggtcagcacctgtactttaaagaagatggccaccaagttaaaggtcagctggtcacccgtacggacggcaaagtgcgttactatgacgcaaattctggcgatcaagcgttcaacaagtccgtgacggttaacggcaaaacgtattacttcggtaatgatggtaccgcgcaaaccgcgggtaacccgaaaggccaaatcttcaaggacggcagcgttctgcgtttctatagcatggaaggccagctggtaattggcagcggctggtattccaacgcgcaaggccaatggctgtatgtgaagaatggtaaagtgttgaccggtttgcagaccgtcggttcccagcgcgtgtactttgatgagaatggcattcaagcaaaaggcaaagcggttcgcacgagcgacggcaaaattcgctacttcgacgagaacagcggtagcatgatcaccaatcaatggaagtttgtttacggtcaatactattactttggtaatgacggtgcggcaatctaccgtggttggaattaa

SEQ ID NO: 26 - protein, 1435 aa, Streptococcus sobrinus
MVDGKYYYYDQDGNVKKNFAVSVGDKIYYFDETGAYKDTSKVDADKSSSAVSQNATIFAANNRAYSTSAENFEAVDNYLTADSWYRPKSILKDGKTWTESGKDDFRPLLMAWWPDTETKRNYVNYMNLVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYEFLLANDVDNSNPVVQAEQLNWLHYLLNFGSIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIDQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAVTNTFIADKTGHWYYLGKDGVAVTGAQTVGKQKLYFEANGEQVKGDFVTSHEGKLYFYDVDSGDMWTDTFIEDKAGNWFYLGKDGAAVSGAQTIRGQKLYFKAYGQQVKGDIVKGTDGKIRYYDAKSGEQVFNKTVKAADGKTYVIGNNGVAVDPSVVKGQTFKDASGALRFYNLKGQLVTGSGWYETANHDWVYIQSGKALTGEQTINGQHLYFKEDGHQVKGQLVTRTDGKVRYYDANSGDQAFNKSVTVNGKTYYFGNDGTAQTAGNPKGQIFKDGSVLRFYSMEGQLVIGSGWYSNAQGQWLYVKNGKVLTGLQTVGSQRVYFDENGIQAKGKAVRTSDGKIRYFDENSGSMITNQWKFVYGQYYYFGNDGAAIYRGWN

SEQ ID NO: 27 - DNA, 4023 bp, Streptococcus salivarius PS4
atgattgacggcaaatactactacgtaaacaaagatggctcgcacaaagagaatttcgcaattaccgtgaatggtcagttgttgtatttcggtaaggacggtgcattgacgtctagcagcacctacagctttacgcagggcaccaccaacatcgttgatggctttagcaaaaacaaccgtgcgtacgattccagcgaggcgagctttgaactgatcgacggttatctgaccgcggactcctggtatcgtccggtgagcattatcaaggacggcgttacgtggcaagccagcaccaaagaggactttcgcccgctgctgatggcctggtggccgaatgttgacacccaggtcaactacctgaattacatgtcgaaggtgtttaacctggacgcgaagtatacgagcaccgacaaacaggttgacctgaatcgcgcagccaaggacattcaggttaagattgagcaaaagattcaggccgagaagagcactcaatggctgcgtgaagcgatttcggccttcgtcaaaacccagccgcagtggaataaagaaacggagaacttctccaagggtggtggtgaggatcatctgcaaggtggtgcactgctgtacgttaacgacccgcgtaccccgtgggctaactccaactaccgcctgctgaatcgtactgcgaccaaccagaccggcacgatcgacaagagcgttctggacgaacagagcgatcctaaccacatgggcggcttcgattttctgctggcgaatgacgtcgataccagcaatccggtggtgcaggcggaacaactgaatcagatccactacctgatgaattggggttccattgttatgggcgacaaagatgcaaacttcgatggtatccgcgtggacgcggtcgataacgttgacgcagatatgctgcaactgtacaccaactactttcgtgagtattatggcgtgaacaaaagcgaggcaaacgctttggcgcacatctcggtgctggaagcgtggagcttgaatgataatcactataatgacaagactgacggtgcggccctggcgatggagaacaaacagcgtttggccctgctgtttagcttggcgaaaccgatcaaagaacgtacccctgcggtgagcccgctgtacaacaacactttcaacacgacgcagcgtgacgaaaagaccgattggattaacaaagacggtagcaaagcctataatgaggacggcaccgtcaagcagtccaccatcggcaagtacaacgagaaatacggcgacgcgtccggcaattatgtgttcattcgcgcccacgataacaacgtccaagacattattgcagagatcattaagaaagaaatcaatccgaaaagcgacggtttcaccattaccgacgccgaaatgaaaaaggcattcgaaatctacaacaaagatatgctgtcctctgataagaaatacaccctgaacaacatcccagcggcctacgcggtgatgctgcaaaacatggaaaccattactcgtgtgtattacggcgatctgtataccgacgatggccattacatggaaaccaagagcccgtactacgacaccattgtgaacctgatgaagaaccgtatcaaatacgtgtccggtggtcaagcgcaacgttcctattggctgccgaccgacggtaagatggataaaagcgatgtcgaactgtatcgcaccaacgaggtgtacaccagcgtccgttacggtaaggacatcatgactgccgatgacacccaaggtagcaagtacagccgtaccagcggtcaggtgaccctggtggtgaacaacccgaagctgtctttggataagagcgcgaagctggacgtcgaaatgggcaagatccatgcaaaccagaaataccgtgctctgatcgtgggtacgccgaacggcatcaaaaacttcacgagcgacgccgaggcaatcgcggctggctacgtgaaagaaaccgacggcaatggtgtgctgaccttcggtgcaaatgacatcaaaggttacgaaacgtttgacatgagcggtttcgttgcagtttgggttccggtaggtgcaagcgatgatcaagacatccgtgtcgccgcaagcaccgcggcaaagaaagaaggtgagctgactttgaaggcaactgaggcgtatgactctcagctgatttacgaaggtttttcgaattttcagaccattccggatggtagcgatccgagcgtttacaccaatcgtaagatcgcggaaaatgttgatttgttcaagagctggggtgtgacctctttcgaaatggcgccacagtttgtgagcgcagacgacggtacgtttctggacagcgttatccagaacggctatgcgtttgcggaccgttatgatctggcgatgtccaaaaacaataagtacggttcgaaagaagatctgcgtaacgcgttgaaggctttgcacaaggccggcatccaagccattgcggactgggttccggatcagatctaccaactgccgggcaaagaagtagtgaccgccactcgtaccgatggtgccggtcgtaagattagcgatgcaattatcgatcacagcctgtacgtcgcaaacagcaagtcgtctggcaaagactatcaagctaaatacggtggtgagttcctggccgagctgaaagcaaagtacccggaaatgtttaaagtcaacatgattagcacgggtaaaccgatcgacgactctgtcaaactgaagcaatggaaggcggagtactttaacggtacgaatgttctggaccgtggtgttggttacgtcctgagcgatgaggcgacgggcaagtactttaccgttacgaaagagggtaactttatcccactgcaattgaaaggtaacgagaaagttatcacgggcttcagctctgacggcaagggcattacctatttcggcacctcgggtaatcaagcgaaaagcgcttttgtcacgttcaatggtaatacctactattttgacgcgcgtggccacatggttaccaacggcgaatatagccctaatggtaaggatgtgtatcgtttcctgccgaatggtattatgttgagcaatgcattctacgttgacggtaacggcaatacctacctgtacaactccaagggccaaatgtacaaaggtggttatagcaaattcgacgttacggaaaccaaagatggtaaagagagcaaagtggtgaaatttcgctactttaccaatgaaggtgtgatggcaaaaggtgttaccgtggtggacggcttcactcaatacttcaacgaagatggcattcagagcaaggacgaactggtgacctacaatggtaaaacctattacttcgaagcgcataccggtaatgcgatcaaaaacacgtggcgcaatatcaagggtaagtggtatcactttgatgcgaatggcgtggcggcaacgggtgcacaggttatcaatggtcagcacctgtactttaatgaggatggttcccaggtgaagggtggcgtcgtgaagaatgcggatggtaccttcagcaagtataaagatggttccggtgacctggtggtcaatgagttcttcactactggtgataacgtgtggtactacgctggtgccaacggcaaaactgtgacgggtgcccaggtcatcaatggccaacacctgtttttcaaagaggacggtagccaggttaagggtgatttcgttaagaacagcgacggcacctactctaagtatgatgcggccagcggcgaacgcctgacgaatgagtttttcacgaccggtgacaaccactggtactatattggtgccaatggcaaaaccgttaccggcgaagtcaagatcggtgatgatacgtacttcttcgcaaaagatggcaagcagctgaagggccagatcgtgacgacccgcagcggtcgtatcagctactacttcggcgactctggtaagaaggcgattagcacctgggtggagattcagccgggtgttttcgtgtttttcgacaaaaatggcctggcatatccgccggaaaacatgaattaa

SEQ ID NO: 28 - protein, 1340 aa, Streptococcus salivarius PS4
MIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 29 - DNA, 4026 bp, Streptococcus salivarius K12
atgacggacggtaaatactattatgtaaatgaggacggcagccacaaagagaatttcgcaattacggtaaacggtcaactgttgtactttggcaaggacggcgctctgacgagcagcagcacgcacagcttcacgccgggtactacgaatattgtggacggtttctcgatcaacaaccgtgcgtacgatagcagcgaagcgagctttgagctgatcaacggttacctgacggcggattcctggtatcgcccggtttctatcatcaaggatggcgtcacgtggcaggcaagcactgccgaggattttcgtccgctgttgatggcctggtggccgaacgttgatacccaggtgaactatctgaactatatgtccaaggtctttaacctggaagccaagtacaccagcaccgataaacaggctgatctgaaccgtgctgcaaaggatatccaggtcaagatcgaacagaagatccaggcggaaaagagcacgcagtggctgcgtgagactatctccgcgtttgttaaaacccagccgcaatggaacaaagagactgagaattactccaagggtggtggcgaagatcatctgcaaggcggtgcgctgttgtacgtgaacgacagccgtaccccgtgggcgaatagcaattaccgcctgctgaatcgcacggcaacgaaccagaccggtaccattaacaagtcggtgttggacgagcaatccgatccaaatcacatgggtggcttcgacttcctgctggcaaacgatgtggatctgagcaatcctgttgtgcaggccgagcagctgaatcaaatccattatctgatgaactggggcagcattgttatgggtgacaaagacgcgaattttgatggtatccgtgtggacgccgttgacaacgtgaacgctgacatgttgcagctgtacacgaactactttcgtgagtattacggcgtcaacaaaagcgaagcgcaagcgctggcgcacattagcgttctggaagcgtggagcttgaacgataaccactataacgacaaaaccgatggtgcggcactggcgatggagaataagcaacgtctggccttgctgttctctctggccaagccgatcaaagatcgtactccggcagtgagcccactgtataacaatactttcaataccacccaacgtgacttcaagacggattggattaacaaggacggtagcaccgcctacaatgaggatggcaccgcgaaacaatctaccatcggtaagtacaatgagaaatatggtgatgcaagcggtaactatgtgtttattcgtgcccatgacaataacgtccaagacattattgcggagatcattaagaaagaaatcaataagaagagcgatggttttaccatcagcgatagcgaaatgaaacaggcgttcgaaatctacaacaaagatatgctgagcagcaataagaaatacactctgaataacattccggcagcgtacgccgtgatgctgcaaaacatggagactatcacccgtgtgtattatggtgacctgtacaccgacgacggtcactatatggaaaccaagagcccgtatcatgacaccattgtgaacctgatgaaaaaccgtatcaagtacgtttctggtggccaggcccaacgctcctattggctgccgaccgacggtaaaatggacaatagcgatgtcgaactgtaccgtactagcgaggtctataccagcgttcgctacggtaaggacattatgacggcggatgacaccgagggtagcaagtactcccgcacgagcggtcaggttaccctggttgttaacaacccgaagctgactctgcatgaaagcgccaaactgaacgtcgagatgggtaagatccacgcaaaccagaaataccgtgcgctgattgtgggtaccgccgatggcatcaaaaactttacgtctgatgccgaagcgatcgcggcaggctacgtaaaagaaacggacagcaatggtgttctgaccttcggcgcaaatgatatcaaaggttacgagactttcgatatgagcggtttcgtcgcagtttgggtgccggtgggtgcgagcgatgatcaggacatccgcgtggcgccgtcgacggaagcgaagaaagaaggtgaactgacgctgaaagccacggaagcgtatgatagccagttgatttatgaaggcttctccaatttccagaccattccggatggcagcgacccgagcgtttataccaaccgcaaaattgctgagaatgttgatctgtttaagtcctggggtgtcactagcttcgaaatggctccgcagtttgtttcggcggacgacggcaccttcctggatagcgttatccagaacggttacgcctttgcggaccgttatgatttggccatgagcaagaacaacaagtacggttctaaagaggatctgcgcgacgcactgaaagcgctgcacaaagctggcattcaggcaatcgcggactgggtcccagaccaaatctaccaactgccaggcaaagaagtggttacggcgacgcgcacggacggtgcgggtcgcaagatcgcggacgccatcattgatcatagcctgtatgttgctaactccaagagctccggtcgcgattaccaagcgcagtatggtggcgagtttctggcagagctgaaagcgaagtacccgaaaatgttcacggaaaacatgattagcacgggtaagccgatcgatgacagcgtcaaactgaagcaatggaaagccaagtatttcaatggtacgaatgtgctggaccgtggtgtcggttacgtcctgtccgacgaggcgaccggcaaatacttcaccgttaccaaagagggtaacttcattccgctgcaactgaccggcaatgaaaaagcggtgaccggtttcagcaacgacggcaagggtatcacctactttggtacgagcggtaatcaggccaagagcgcgttcgtcacctttaacggcaatacgtactatttcgacgcgcgtggccacatggtcacgaacggcgagtatagcccgaacggcaaagatgtctaccgttttctgccaaatggtattatgttgtcgaacgcgttttatgtcgacgcaaacggtaatacgtacttgtacaactacaagggccagatgtacaaaggtggttatacgaaatttgatgtcaccgaaactgataaagatggtaatgagagcaaggtggtcaagtttcgttatttcaccaatgagggcgtcatggctaagggtctgaccgtcattgacggtagcacccagtactttggtgaggatggttttcaaacgaaggacaagctggcgacctataaaggtaagacttattacttcgaggcacacacgggcaatgcgatcaaaaacacctggcgtaacatcgacggtaagtggtatcacttcgatgagaatggcgttgccgcgaccggtgcacaagtgattaacggtcaaaaactgtatttcaacgaggatggctcgcaagtgaagggcggtgttgttaagaacgccgacggtacctacagcaaatacaaagagggcagcggtgagctggttaccaacgagtttttcacgaccgacggtaatgtgtggtactatgctggtgcggatggcaagactgtgaccggtgctcaggtcattaatggtcagcacctgtactttaaagaagatggcagccaggtgaaaggtggtgtggtgaaaaacgcggacggtacgtacagcaagtatgacgccgccaccggtgaacgcttgaccaatgagttctttaccacgggcgataacaattggtactatattggttctaatggtaagaccgtaaccggtgaagtcaaaatcggtgcggacacctattactttgccaaagatggcaaacaggtcaagggccaaaccgtcaccgcaggcaatggccgcatctcctattactacggcgattctggtaagaaagcaatcagcacgtggatcgaaattcaaccgggtatctatgtctattttgataagacgggcatcgcgtacccaccgcgtgtgctgaattaa

SEQ ID NO: 30 - protein, 1341 aa, Streptococcus salivarius K12
MTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 31-32 - skipped (2 entries)

SEQ ID NO: 33 - DNA, 4026 bp, Streptococcus salivarius JIM8777
atgatcgacggcaaatactactatgtaaacgaggacggcagccacaaagagaatttcgcgattacggtaaacggtcagctgctgtactttggtaaggacggtgctctgacgagcagctccacgtacagctttaccccgggtacgaccaatattgtcgatggcttcagcattaacaaccgtgcgtatgacagcagcgaggcatcctttgagctgatcgatggttatttgaccgcggatagctggtatcgtccggcgagcatcattaaggacggcgttacgtggcaggcctcgaccgcagaagattttcgtccgctgctgatggcttggtggccgaatgttgacacccaggtgaattatctgaattacatgtccaaggttttcaacctggatgcaaagtacaccagcaccgacaagcaggaaaccctgaacgtggctgcgaaagatatccaagtcaagattgagcaaaagattcaggcagagaaatctacccagtggctgcgtgaaacgattagcgcgtttgttaaaactcagccgcaatggaataaagaaacggaaaactattccaagggtggtggcgaggaccatctgcaaggcggtgccctgttgtacgttaacgattcgcgcaccccgtgggcgaactcgaactatcgcttgctgaaccataccgctaccaatcaaaaaggcactattgacaaatctgtcctggacgagcagagcgacccgaaccacatgggcggtttcgattttctgctggcgaacgacgtcgacctgagcaacccggtggtgcaggccgaacaactgaaccagattcactacctgatgaattggggtagcatcgtgatgggtgataaagatgcgaactttgacggcattcgtgtcgatgcggtcgataacgtggacgccgacatgttgcagctgtacacgaactactttcgtgagtactacggcgttaacaagagcgaagcaaatgccctggcgcatatcagcgttctggaagcgtggagcctgaatgacaatcactataacgataagacggacggtgcggccctggcaatggagaataaacaacgtctggcgctgctgttcagcctggcgaaaccgatcaaagagcgtacgccggctgtgagcccactgtataacaacaccttcaatactacgcagcgtgacgagaaaacggactggattaacaaagacggtagcaaagcgtataacgaggatggtaccgtcaagcaatcgaccattggtaagtacaatgagaagtatggcgacgcaagcggtaattacgtgttcattcgtgcccacgacaacaatgttcaagacatcatcgccgaaatcatcaagaaagagatcaaccctaagagcgacggtttcaccatcaccgacgcagagatgaagaaggcctttgaaatctacaacaaggacatgttgagcagcgataagaagtatactctgaacaacattccggctgcgtacgcggtgatgttgcagaatatggaaaccatcacgcgtgtttactatggtgatctgtataccgataatggcaactacatggaaacgaaaagcccgtactatgacaccattgttaatctgatgaagaatcgcatcaagtatgtgtctggcggtcaagcgcagcgttcttactggctgccgaccgatggtaagatggacaatagcgatgtggaactgtaccgcaccaacgaggtatacgcttctgtgcgctatggtaaagacattatgaccgccgatgataccgagggttccaagtactcccgtacgagcggccaagttaccttggtggcaaacaacccgaaattgaccctggaccaaagcgcgaaactgaaagtggagatgggtaagatccacgcaaatcaaaagtaccgtgcactgattgtcggtaccgccgacggtatcaagaatttcaccagcgatgcggatgcgattgcagcaggctatgttaaagagactgatagcaatggtgtgctgacgtttggtgcgaacgacattaaaggctatgaaacgtttgacatgagcggtttcgttgcggtgtgggtgcctgtgggtgctagcgatgatcaggatatccgtgtcgcgccgagcaccgaggcaaagaaagaaggtgagctgacgttgaaagcgaccgaggcctatgacagccagttgatttacgaaggtttcagcaatttccaaaccattccagacggttccgatccgagcgtctacaccaatcgcaaaatcgcggaaaacgttgatctgttcaaaagctggggtgtgaccagcttcgaaatggcaccgcaattcgttagcgcggacgatggtacgttcttggacagcgttatccaaaatggctatgcgttcgccgatcgttatgacttggcgatgagcaaaaacaacaaatacggcagcaaagaggatctgcgcgacgccctgaaagcgctgcataaagcgggtattcaagccatcgctgactgggttccggaccagatctaccagctgccgggtaaagaagtcgttaccgcgacccgcaccgatggcgctggccgtaagatcgcggatgcaattatcgatcatagcttgtatgtggccaatactaaaagctccggtaaggattaccaggcgaaatatggtggtgaatttctggctgagctgaaggccaaatacccggagatgttcaaggtcaacatgattagcaccggcaaacctattgatgactctgtcaaattgaaacaatggaaggcagagtatttcaatggcactaacgtcctggaacgtggtgttggttacgtgctgagcgacgaggcgaccggtaaatacttcaccgttacgaaggacggcaatttcatcccgctgcaactgaccggtaatgagaaggttgtgacgggtttttctaatgacggtaagggcattacctacttcggtacctcgggtacccaggcaaagagcgcattcgtgacgtttaacggtaacacctactactttgatgcacgcggccacatggtgacgaacggcgagtacagcccgaacggcaaggatgtttatcgcttcctgccgaatggcatcatgctgtccaatgcgttttacgtcgatgcaaatggtaatacttacctgtacaacagcaagggtcagatgtataagggcggttataccaagttcgacgttactgaaacggacaaggacggtaaagagagcaaagtagtgaagtttcgttatttcacgaacgaaggcgtcatggcgaaaggtgtcaccgttattgatggctttacccagtatttcggtgaagatggctttcaagcgaaggacaagctggtgacctttaagggcaaaacctactattttgacgcgcacacgggcaacgccatcaagaacacctggcgtaatatcgacggtaagtggtatcattttgatgcgaacggtgtggcggcgaccggcgcacaggtcattaatggtcaaaaactgtactttaatgaggacggtagccaagtcaaaggtggcgtcgtcaagaatgcagatggcacctatagcaaatacaaagagggctccggtgagctggttaccaacgagttctttaccacggatggtaacgtctggtactatgctggtgcgaatggcaagaccgttaccggtgcacaggttatcaacggccagcacctgtacttcaatgcggatggctctcaagtgaagggcggtgtcgtcaaaaacgcggacggtacgtactccaaatacgatgccgcgaccggtgaacgtctgaccaatgagtttttcacgactggtgacaacaattggtactacatcggcgccaacggtaagacggttacgggcgaagtgaaaattggcgacgatacgtactacttcgcaaaagatggtaaacaggtgaaaggtcagacggtttccgctggtaatggccgcatcagctactattacggtgactctggtaaacgtgcggttagcacgtgggttgaaattcaaccgggcgtgtatgtctattttgataagaatggcctggcatatccaccgcgcgttttgaattaa

SEQ ID NO: 34 - protein, 1341 aa, Streptococcus salivarius JIM8777
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYTSTDKQETLNVAAKDIQVKIEQKIQAEKSTQWLRETISAFVKTQPQWNKETENYSKGGGEDHLQGGALLYVNDSRTPWANSNYRLLNHTATNQKGTIDKSVLDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDNGNYMETKSPYYDTIVNLMKNRIKYVSGGQAQRSYWLPTDGKMDNSDVELYRTNEVYASVRYGKDIMTADDTEGSKYSRTSGQVTLVANNPKLTLDQSAKLKVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKNTWRNIDGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDAATGERLTNEFFTTGDNNWYYIGANGKTVTGEVKIGDDTYYFAKDGKQVKGQTVSAGNGRISYYYGDSGKRAVSTWVEIQPGVYVYFDKNGLAYPPRVLN

SEQ ID NO: 35-58 - skipped (24 entries)

SEQ ID NO: 59 - protein, 1242 aa, Streptococcus sp. C150
MINGKEYYVEDDGTVRKNYVLERNGGSQYFNAETGELSNQKDYRFDKNGGTGSAADSTTNTNVTVNGDKNAFYGTTEKDIELVDGYFTANTWYRPKEILKDGKEWTASTENDKRPLLTVWWPSKAIQASYLNYMREEGLGTNQTFTSYSSQTQMDQAALEVQKRIEERIAREGNTDWLRTTIKNFVKTQPGWNSTSENLDNSDHLQGGALLYNNSNRTSYANSDYRLLNRTPTQQDGTRRYFKDNSSGGFEFLLANDIDNSNPAVQAEQLNWLHYIMNIGSLTGGSEDENFDGVRVDAVDNVNADLLQIASDYFKAKYGVEKSEEEAIKHLSILEAWSHNDAYYNEDTKGAQLPMDDPLRLAMVFSFLRPIGNRSGLEPLITNSLNDRSESKKNTKRMANYTFVRAHDSEVQSVIGQIIKNEINPQSTGNTFTLDEMKKAFKIYNADMRSANKRYTQYNIPSAYAFMLTNKDTVPRVYYGDLYTDDGQYMAQKSPYHDAISTLLQARIRYAAGGQDMKMSYVGSGNTNGWDASGVLTSVRYGKGANNASDAGTAETRNQGMAVILSNQPALRLNSNLTINMGAAHRNQAYRPLLLTTSNGVASYLNDGDANGIVKYTDANGYLTFNPGEISGVRNAQVDGYLAVWVPLGASENQDVRVAASKSKNSSGLVYDSSAALDSQVIYEGFSNFQDFVQDPSQYTNKKIAENANLFKSWGITSFEFAPQYVSSDDGTFLDSVIQNGYAFSDRYDIGMSKDNKYGSLADLKAALKSLHAVGISAIADWVPDQIYNLPGDEVVTATRVNNYGETKDGAIIDHSLYVAKTRTFGNDYQGKYGGAYLDELKRLYPQFFDRVQISTGKRLTTDEKITKWSAKYMNGTNILDRGSEYVLKNGLSGYYGTNGGKVSLPKVVGSNQSTNNNNQNGDGSGRFEKSWGSVYYRYNDGQRARNAFIKDNDGNVYYFDNTGRMAIGEKTIDGKQYFFLANGVQLRDGYRQNRRGQVFYYDENGIMSQTGKPSPKPEPKPDNNTFSRNQFIQIGNNVWAYYDGNGKRVIGRQNINGQELFFDNNGVQVKGRTAQVDGVTRYFDANSGEMARNRFAEVEPGVWAYFNNDGAAVTGSQNINGQTLYFDQNGHQVKGALVTVDGNLRYYDANSGDLYRNRFQEVNGSWYYFDGNGNAVKGMVNINGQNLLFDNDGKQVKGHLVRVNGVIRYYDPNSGEMAVNRWVEISSGWWVYFDGEGRGQI

SEQ ID NO: 60 - protein, 1518 aa, Streptococcus salivarius
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 61 - protein, 1528 aa, Streptococcus salivarius K12
MTNKITGKIIMENKVHYKLHKVKKQWVTIAVASAALATVVGGLSATTSSVSADETQDKIVTQPNLDTTADLVTSTEATKEVDKRTNTKEADVLTPAKETNAVETATTTNTQATAEAATTATTSDVAVAAVPNKEAVVTTDAPAVTTEKAEEQPATVKAEVVNTEVKAPQAALKDSEVEAALSLKNIKYTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 62 - protein, 1518 aa, Streptococcus salivarius SK126
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 63 - protein, 1431 aa, Streptococcus salivarius PS4
MTKETNTVDAATTTNTQAAADAATKTADAAVTALPNKEAVVTTDAPAVTTEKAAEQPATVKSEVVNTEVKAPEAALKDSEVEAALSLKNIKNIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 64 - protein, 1532 aa, unknown
  unknown Streptococcus sp. C150
MENKVHYKLHKVKKQWVTIAVASAALATVVGGLSATTSSVSADETQDKTVTQPNSDTTADLVTSTEATKEVDKRTNTKEADVLTPAKETNTVETAATTNTQATAEAAKTATTTNTQATAEVAKTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 65 - protein, 1341 aa, Streptococcus salivarius
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN